(19) United States
(12) Patent Application Publication
(10) Pub. No.: US 2017/0362621 A1
(43) Pub. Date: Dec. 21, 2017

(54) ENGINEERED MULTIFUNCTIONAL ENZYMES AND METHODS OF USE

(71) Applicant: Danisco US Inc., Palo Alto, CA (US)

(72) Inventors: Zachary Q. Beck, Palo Alto, CA (US); Meredith K. Fujdala, Palo Alto, CA (US); Henrik Hansson, Uppsala (SE); Thijs Kaper, Palo Alto, CA (US); Slavko Kralj, Palo Alto, CA (US); Amy D. Liu, Palo Alto, CA (US); Nils Egil Mikkelsen, Uppsala (SE); Mats Sandgren, Uppsala (SE)

(21) Appl. No.: 15/536,915

(22) PCT Filed: Dec. 18, 2015
(86) PCT No.: PCT/US15/66710

§ 371 Date: Jun. 16, 2017

Related U.S. Application Data
(60) Provisional application No. 62/093,650, filed on Dec. 18, 2014.

(51) Int. Cl. C12P 19/14; C12N 9/24; C12P 19/02; C12N 9/42

(52) U.S. Cl. CPC C12P 19/14; C12Y 302/01037; C12Y 302/01021; C12N 9/2445; C12N 9/248; C12P 19/02

(57) ABSTRACT

Provided are certain glycosyl hydrolase family 3 (GH3) beta-glucosidase enzymes engineered to acquire beta-xylosidase activities. Provided also are compositions comprising multi-functional GH3 enzymes and methods of use or industrial applications thereof.

### CROSS-REFERENCE TO RELATED APPLICATIONS

This application claims the benefit of priority from U.S. Provisional Patent Application Ser. No. 62/093,650, filed in the United States Patent and Trademark Office on Dec. 18, 2014, the entirety of which is herein incorporated by reference.

### FIELD OF THE INVENTION

The present compositions and methods relates to certain glycosyl hydrolase family 3 enzymes engineered to confer a new and different enzymatic activity. Such enzymes and compositions are useful and beneficial for hydrolyzing lignocellulosic biomass material into fermentable sugars.

### REFERENCE TO SEQUENCE LISTING SUBMITTED ELECTRONICALLY

The content of the electronically submitted sequence listing in ASCII text file (Name: NB40830WOPCT_Seq_List_ST25; Size: 195,386 bytes, and Date of Creation: Nov. 20, 2015) filed with the application is incorporated herein by reference in its entirety.

### BACKGROUND

Cellulose and hemicellulose are the most abundant plant materials produced by photosynthesis. They can be degraded and used as an energy source by numerous microorganisms (e.g., bacteria, yeast and fungi) that produce extracellular enzymes capable of hydrolysis of the polymeric substrates to monomeric sugars (Aro et al., (2001) J. Biol. Chem., 276: 24309-24314). As the limits of non-renewable resources approach, the potential of cellulose to become a major renewable energy resource is enormous (Krishna et al., (2001) Bioresource Tech., 77: 193-196). The effective utilization of cellulose through biological processes is one approach to overcoming the shortage of foods, feeds, and fuels (Ohmiya et al., (1997) Biotechnol. Gen. Engineer Rev., 14: 365-414).

Most of the enzymatic hydrolysis of lignocellulosic biomass materials focus on cellulases, which are enzymes that hydrolyze cellulose (comprising beta-1,4-glucan or beta D-glucosidic linkages) resulting in the formation of glucose, cellobiose, cellooligosaccharides, and the like. Cellulases have been traditionally divided into three major classes: endoglucanases (EC 3.2.1.4) (“EG”), exoglucanases or cellobiohydrolases (EC 3.2.1.91) (“CBH”) and beta-glucosidases ([beta]-D-glucoside glucohydrolase; EC 3.2.1.21) (“BG”) (Knowles et al., (1987) TIBTECH 5: 255-261; and Schulein, (1988) Methods Enzymol., 160: 234-243). Endoglucanases act mainly on the amorphous parts of the cellulose fiber, whereas cellobiohydrolases are also able to degrade crystalline cellulose (Nevalainen and Penttila, (1995) Mycota, 303-319). Thus, the presence of a cellobiohydrolase in a cellulase system is required for efficient solubilization of crystalline cellulose (Suurnakki et al., (2000) Cellulose, 7: 189-209). Beta-glucosidase acts to liberate D-glucose units from cellobiose, cello-oligosaccharides, and other glucosides (Freer, (1993) J. Biol. Chem., 268: 9337-9342).

In order to obtain useful fermentable sugars from lignocellulosic biomass materials, however, the lignin will typically first need to be permeabilized, for example, by various pretreatment methods, and the hemicellulose disrupted to allow access to the cellulose by the cellulases. Hemicelluloses have a complex chemical structure and their main chains are composed of mannans, xylans and galactans.

Enzymatic hydrolysis of the complex lignocellulosic structure and rather recalcitrant plant cell walls involves the concerted and/or tandem actions of a number of different endo-acting and exo-acting enzymes (e.g., cellulases and hemicellulases). Beta-xylanases and beta-mannanases are endo-acting enzymes, beta-mannosidase, beta-glucosidase and alpha-galactosidases are exo-acting enzymes. To disrupt the hemicellulose, xylanases together with other accessory proteins (non-limiting examples of which include L-α-arabinofuranosidases, feruloyl and acetylxylan esterases, glucuronidases, and β-xylosidases) can be applied.

A number of commercial enzymes products have been available to a nascent industry of producing cellulosic fuels and other biochemicals from cellulosic biomass sources. However, because large amounts and great variety of such enzymes are typically required, acting in consortium, to convert the complex lignocellulosic structures of such plant-based materials, the costs associated with producing and reliably supply such enzymes remains a key bottleneck to commercial viability. Microorganisms such as, for example, celluloytic bacterial and fungal organisms have been engineered and used to produce such panels of enzymes, typically in mixtures. However it has been recognized that the extent or the capacity to which microorganisms can be engineered to produce enzymes is not limitless, and increasing the levels of one or more enzymes, for example, cellulases, can come at the expense of the productivities of other enzymes also required for achieving effective cellulosic conversion.

Thus creating and discovering enzymes that can execute multiple functionalities is not only helpful for providing or supplementing to the suite of activities, but also boosts, albeit indirectly, the production and yield of other enzyme activities by host microorganisms. This is especially the case if the engineered multifunctional enzymes acquire not only the added useful activity, but other beneficial characteristics such as increased stability, broader or more targeted substrate specificity. These enzymes, when included in the enzyme products, have the potential of improving the hydrolysis performance of the enzyme mixtures, reducing cost of production, and may also help to achieve more reliable supply of enzyme products simply because lesser number of enzymes will need to be produced by the engineered organism.

### SUMMARY

One aspect of the present compositions and methods relates to the engineering of a beta-glucosidase glycosyl hydrolyase family 3 (GH3) enzyme, into a multifunctional enzyme having not only beta-glucosidase activity but also beta-xylosidase activity. Specifically the engineered beta-xylosidase GH3 enzyme comprises a polypeptide sequence having at least 35% (e.g., at least 35%, at least 40%, at least 45%, at least 50%, at least 55%, at least 60%, at least 65%, at least 70%, at least 75%, at least 80%, at least 85%, at least 90%, at least 95%, or higher) identity to SEQ ID NO:2 (Trichoderma reesei Bgl1), with one or more substitutions at positions 43, 237 and 255, wherein the positions are numbered in reference to the mature sequence of Bgl1, SEQ ID NO:3. Suitable polypeptide sequences which may comprise one or more substitutions at positions 43, 237 and 255 include polypeptide sequences having at least 35% (e.g., at least 35%, at least 40%, at least 45%, at least 50%, at least 55%, at least 60%, at least 65%, at least 70%, at least 75%, at least 80%, at least 85%, at least 90%, at least 95%, or higher) identity to SEQ ID NO: 37, 38, 39, 41, 43, 44, 45, 46, 47, 48, 49, 50, 51, 53, 54, 55, 56, or 57 wherein the positions are numbered in reference to the mature sequence of Bgl1, SEQ ID NO:3. In embodiments, such polypeptides comprise a substitution of a valine residue at position 43 with a tryptophan (W), phenylalanine (F), or leucine (L), wherein the positions are numbered in reference to the mature sequence of Bgl1, SEQ ID NO:3. Accordingly, provided herein are polypeptides having the amino acid sequence of SEQ ID NO: 37, 38, 39, 41, 43, 44, 45, 46, 47, 48, 49, 50, 51, 53, 54, 55, 56, or 57 and further comprising, for example, a substitution of a valine residue at position 43 with a leucine, wherein the position is numbered in reference to SEQ ID NO: 3.

In some embodiments, the engineered beta-glucosidase of the first aspect is one that comprises an amino acid sequence of at least 50% identity (e.g., at least 50%, at least 55%, at least 60%, at least 65%, at least 70%, at least 75%, at least 80%, at least 85%, least 90%, at least 91%, at least 92%, at least 93%, at least 94%, at least 95%, at least 96%, at least 97%, at least 98%, or at least 99% identity) to SEQ ID NO:2 with one or more substitutions at the enumerated positions.

In certain embodiments, at least one of the substitutions is the replacement of a valine (V) residue at position 43 with a tryptophan (W), phenylalanine (F), or leucine (L).

In certain embodiments, at least one of the substitutions is the replacement of a tryptophan (F) residue at position 237 with a leucine (L), isoleucine (I), valine (V), alanine (A), glycine (G) or cysteine (C).

In certain embodiments, at least one of the substitutions is the replacement of a methionine (M) residue at position at position 255 with a cysteine (C).

In some embodiments, the engineered beta-glucosidase of the first aspect is one that comprises an amino acid sequence of at least 50% identity (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity) to SEQ ID NO:2 with two or more substitutions at the enumerated positions.

In certain embodiments, the two or more substitutions are at positions 43 and 237. Alternatively the two or more substitutions are at positions 43 and 255. Furthermore, the two or more substitutions can be at positions 237 and 255. In some particular embodiments, the substitutions are at all three positions, namely positions 43, 237 and 255.

In any of the embodiments described above, the substitutions at position 43 may be with a tryptophan (W), phenylalanine (F), or leucine (L). The substitutions at position 237 may be with a leucine (L), isoleucine (I), valine (V), alanine (A), glycine (G) or cysteine (C). The substitution at position 255 may be with a cysteine.

In some embodiments, the engineered beta-glucosidase may be one comprising a polypeptide having an amino acid sequence that is at least 35% identity (e.g., at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% identity) to SEQ ID NO:2, with the substitutions V43W/F/L, V43W/W237L, V43W/W237I, V43W/W237V, V43W/W237G, V43F/W237L, V43F/W237I, V43F/W237V, V43F/W237A, V43F/W237G, V43L/W237L, V43L/W237I, V43L/W237V, V43L/W237A, V43L/W237G, V43W/W237C/M255C, V43F/W237C/M255C, or V43L/W237C/M255C. In certain embodiments, the engineered beta-glucosidase has detectable beta-xylosidase activity. In some embodiments, the engineered beta-glucosidase has at least 2% (e.g., at least 5%, at least 10%, at least 15%, or at least 20% or higher) of the beta-xylosidase activity of purified Trichoderma reesei beta-xylosidase 3A (Xyl3A) as measured using a standard assay measuring the hydrolysis of model substrate para-nitrophenol-beta-D-xyloside (pNpX). In some embodiments, the engineered beta-glucosidase has at least 2% higher (e.g., at least 2% higher, at least 5% higher, at least 10% higher, at least 15% higher, or even at least 20% higher) beta-xylosidase activity as compared to that of the native, unengineered, parent beta-glucosidase.

In some embodiments, the engineered beta-glucosidase has at least 2% (e.g., at least 5%, at least 10%, at least 15%, or at least 20% or higher) of the beta-xylosidase activity of purified Trichoderma reesei beta-xylosidase 3 (Xyl3A) as measured using a standard assay measuring xylosidase activity. In certain particular embodiments, the engineered beta-glucosidase has at least 2% higher (e.g., at least 2% higher, at least 5% higher, at least 10% higher, at least 15% higher, or even at least 20% higher) beta-xylosidase activity than that of the native, unengineered, parent beta-glucosidase. In some embodiments, the engineered beta-glucosidase retains substantial level of beta-glucosidase activity, for example, at least 95%, at least 90%, at least 85%, at least 80%, at least 75%, at least 70%, at least 65%, at least 60%, at least 55%, at least 50%, at least 45%, at least 40%, at least 35%, or at least 30%, of its parent unengineered beta-glucosidase, while acquiring increased beta-xylosidase activity. In certain particular embodiments, the engineered beta-glucosidase has not only retained all beta-glucosidase activity of its parents, acquired additional beta-xylosidase activity, but has increased beta-glucosidase activity as compared to its parent.

In a related second aspect, the engineered beta-glucosidase having also beta-xylosidase activity, is encoded by a polynucleotide having at least about 35% identity (e.g., at least about 35% identity, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%) to SEQ ID NO:1, whereby the polynucleotide also encodes certain substitution amino acid residues at positions 43, 237 and 255, with reference to the mature Trichoderma reesei Bgl1 amino acid sequence of SEQ ID NO:3. In some embodiments, the engineered beta-glucosidase has at least 2% (e.g., at least 5%, at least 10%, at least 15%, or at least 20% or higher) of the beta-glucosidase activity of purified Trichoderma reesei beta-xylosidase 3 (Xyl3A) as measured using a standard assay measuring the hydrolysis of model substrate para-nitrophenol-beta-D-xyloside (pNpX). In certain embodiments, the engineered beta-glucosidase has at least 2% higher (e.g., at least 2% higher, at least 5% higher, at least 10% higher, at least 15% higher, or even at least 20% higher) beta-xylosidase activity as compared to that of the native, unengineered, parent beta-glucosidase. In some embodiments, the engineered beta-glucosidase retains substantial level of beta-glucosidase activity, for example, at least 95%, at least 90%, at least 85%, at least 80%, at least 75%, at least 70%, at least 65%, at least 60%, at least 55%, at least 50%, at least 45%, at least 40%, at least 35%, or at least 30%, of its parent unengineered beta-glucosidase, while acquiring increased beta-xylosidase activity. In certain particular embodiments, the engineered beta-glucosidase has not only retained all beta-glucosidase activity of its parents, acquired additional beta-xylosidase activity, but has increased beta-glucosidase activity as compared to its parent, for example, by about 5%, by about 10%, or even by about 15%.

In some embodiments, the engineered beta-glucosidase is encoded by a polynucleotide having at least 35% identity (e.g., at least about 35% identity, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%) to SEQ ID NO:1, whereby the polynucleotide also encodes one of the following substitutions: V43W/F/L, V43W/W237L, V43W/W237I, V43W/W237V, V43W/W237G, V43F/W237L, V43F/W237I, V43F/W237V, V43F/W237A, V43F/W237G, V43L/W237L, V43L/W237I, V43L/W237V, V43L/W237A, V43L/W237G, V43W/W237C/M255C, V43F/W237C/M255C, or V43L/W237C/M255C, the numbering of the residues being in reference to SEQ ID NO:3. In some embodiments, the engineered beta-glucosidase has at least 2% (e.g., at least 5%, at least 10%, at least 15%, or at least 20% or higher) of the beta-xylosidase activity of purified Trichoderma reesei beta-xylosidase 3 (Xyl3A) as measured using a standard assay measuring the hydrolysis of model substrate para-nitrophenol-beta-D-xyloside (pNpX). In some embodiments, the engineered beta-glucosidase has at least 2% (e.g., at least 5%, at least 10%, at least 15%, or at least 20% or higher) of the beta-xylosidase activity of purified Trichoderma reesei beta-xylosidase 3 (Xyl3A) as measured using a standard assay measuring the xylosidase activity. In certain embodiments, the engineered beta-glucosidase has at least 2% higher (e.g., at least 2% higher, at least 5% higher, at least 10% higher, at least 15% higher, or even at least 20% higher) beta-xylosidase activity as compared to that of the native, unengineered, parent beta-glucosidase. In certain particular embodiments, the engineered beta-glucosidase has not only retained all beta-glucosidase activity of its parents, acquired additional beta-xylosidase activity, but has increased beta-glucosidase activity as compared to its parent, for example, by about 5%, by about 10%, by about 15% or more. In other words, the resulting multifunctional engineered enzyme not only acquired an additional beta-xylosidase activity but also is a better, or more superior beta-glucosidase than the parent enzyme, for example, one that has higher beta-glucosidase activity, one that has broader pH activity profile more suitable for hydrolysis of lignocellulosic biomass substrates, one that has higher thermoactivity, one that has reduced or is less susceptible to product inhibition, etc.

In certain embodiments, the engineered beta-glucosidase is encoded by a polynucleotide having at least 35% (e.g., at least about 35% identity, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%) identity to SEQ ID NO:1, or hybridizes under medium stringency conditions, high stringency conditions, or very high stringency conditions to SEQ ID NO:1, or to a complementary sequence thereof, whereby the polynucleotide also encodes certain amino acid substitutions at residues 43, 237 and 255 of SEQ ID NO:3. In some embodiments, the amino acid substitution is selected from one of the following: V43W/F/L, V43W/W237L, V43W/W237I, V43W/W237V, V43W/W237G, V43F/W237L, V43F/W237I, V43F/W237V, V43F/W237A, V43F/W237G, V43L/W237L, V43L/W237I, V43L/W237V, V43L/W237A, V43L/W237G, V43W/W237C/M255C, V43F/W237C/M255C, or V43L/W237C/M255C. In some embodiments, the engineered beta-glucosidase has at least 2% (e.g., at least 5%, at least 10%, at least 15%, or at least 20% or higher) of the beta-xylosidase activity of purified Trichoderma reesei beta-xylosidase 3 (Xyl3A) as measured using a standard assay measuring the hydrolysis of model substrate para-nitrophenol-beta-D-xyloside (pNpX). In certain embodiments, the engineered beta-glucosidase has at least 2% higher (e.g., at least 2% higher, at least 5% higher, at least 10% higher, at least 15% higher, or even at least 20% higher) beta-xylosidase activity as compared to that of the native, unengineered, parent beta-glucosidase. In some embodiments, the engineered beta-glucosidase retains substantial level of beta-glucosidase activity, for example, at least 95%, at least 90%, at least 85%, at least 80%, at least 75%, at least 70%, at least 65%, at least 60%, at least 55%, at least 50%, at least 45%, at least 40%, at least 35%, or at least 30%, of its parent unengineered beta-glucosidase, while acquiring increased beta-xylosidase activity. In certain particular embodiments, the engineered beta-glucosidase has not only retained all beta-glucosidase activity of its parents, acquired additional beta-xylosidase activity, but has increased beta-glucosidase activity as compared to its parent, for example, by about 5%, by about 10%, by about 15% or more. In other words, the resulting multifunctional engineered enzyme not only acquired an additional beta-xylosidase activity but also is a better, or superior beta-glucosidase as compared to the parent enzyme, for example, is one that has higher beta-glucosidase activity than the parent enzyme, is one that has broader or more suitable pH activity profile for lignocellulosic biomass hydrolysis, is one that has higher thermoactivity, is one that has reduced or is less affected by product inhibition, etc.

In some embodiments, the engineered beta-glucosidase of the first and second aspects further comprises a native or non-native signal peptide such that it is produced or secreted by a host organism, for example, the signal peptide comprises a sequence that is at least 90% identical to any one of SEQ ID NOs:8-36 to allow for heterologous expression in a variety of fungal host cells, yeast host cells and bacterial host cells. Accordingly in some embodiments, the enzyme is encoded by a polynucleotide or isolated nucleic acid comprising a sequence that is at least 35% (e.g., at least about 35% identity, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%) identical to SEQ ID NO:1, but which polypeptide also comprises an amino acid substitution at residues 43, 237 and 255 of SEQ ID NO:3. In some embodiments, the polynucleotide sequence also comprises a nucleic acid sequence encoding a signal peptide sequence, for example, one selected from SEQ ID NOs:8-36.

Accordingly embodiments of the present compositions and methods include an expression vector comprising the isolated nucleic acid as described above in operable combination with a regulatory sequence. In some embodiments, the regulatory sequence and the sequence of the engineered beta-glucosidase GH3 enzyme having both beta-glucosidase and beta-xylosidase activities are derived from different microorganisms.

Also embodiments of the present compositions and methods include a host cell comprising the expression vector. In certain embodiments, the host cell is a bacterial cell or a fungal cell.

In a related embodiment, the compositions and methods of the present disclosure include a composition comprising the host cell described above and a culture medium. Embodiments of the present compositions and methods include a method of producing an engineered beta-glucosidase polypeptide that has both beta-glucosidase activity and beta-xylosidase activity, and in certain particular embodiments, even higher or better beta-glucosidase activity, comprising: culturing the host cell described above in a culture medium, under suitable conditions to produce the multifunctional enzyme. Accordingly the present compositions and methods also include a composition comprising an engineered beta-glucosidase enzyme having both beta-glucosidase and beta-xylosidase activity, and in certain particular embodiments, higher beta-glucosidase activity even than the parent non-engineered enzyme, broader or more suitable pH activity profile, higher thermoactivity or less susceptible to product inhibition, in the supernatant of a culture medium produced in accordance with the method for producing the enzyme as described above.

In further embodiments, the engineered beta-glucosidase GH3 enzyme having both beta-glucosidase and beta-xylosidase activity is one heterologously expressed by a host cell. In some embodiments, the polypeptide is co-expressed with one or more cellulase genes. In some embodiments, the polypeptide is co-expressed with one or more other hemicellulase genes. In some further embodiments, the polypeptide is co-expressed with one or more cellulases genes and one or more hemicellulase genes.

In a related third aspect, it is provided a composition comprising the engineered GH3 polypeptide, which has both beta-glucosidase activity and beta-xylosidase activity as described in the above embodiments. In some particular embodiments, the engineered GH3 polypeptide has not only acquired a new, beta-xylosidase activity but also retained substantial level of or even has higher level of beta-glucosidase activity than that of its parent unengineered GH3 polypeptide. In some embodiments, the composition comprises further one or more cellulases, including for example, one or more endoglucanases, one or more cellobiohydrolases, and one or more other enzymes having beta-glucosidase activity. In some embodiments, the composition further comprises one or more hemicellulases, including for example, one or more L-alpha-arabinofuranosidases, one or more xylanases, and one or more other enzymes having beta-xylosidase activities. In some embodiments, the composition further comprises, beside the engineered GH3 polypeptide having both beta-glucosidase activity and beta-xylosidase activity, one or more cellulases and one or more hemicellulases.

In certain embodiments, the composition of the third aspect is a fermentation broth of a host cell engineered to express the engineered beta-glucosidase GH3 polypeptide that has both beta-glucosidase activity and beta-xylosidase activity as provided herein. In some embodiments, the composition is a supernant of a fermentation broth of a suitable host cell subject to minimum or no post-production processing including, without limitation, filtration to remove cell debris, cell-kill procedures, and/or ultrafiltration or other steps to enrich or concentrate the enzymes therein.

In a related fourth aspect, a method of using the composition of the third aspect is provided. The composition comprising the engineered beta-glucodiase GH3 enzyme having both beta-glucosidase and beta-xylosidase activities is used to hydrolyze or break down a lignocellulosic biomass substrate. In some embodiments, the lignocellulosic biomass substrate is subject to a suitable pretreatment step prior to be being placed in contact with the composition of the third aspect. In certain embodiments, the composition of the third aspect is placed in contact with the lignocellulosic biomass subject under suitable conditions and for sufficient time period to allow the conversion of cellulose and hemicelluloses components of the biomass substrate into fermentable sugars. In some embodiments, a suitable ethanologen microorganism can be employed to convert such fermentable sugars into bioethanol or other biochemicals.

In a further aspect, the engineered GH3 enzyme having both beta-glucosidase activity and beta-xylosidase activity as provided in the above aspects and embodiments provides certain internal reciprocal synergy in that lesser or reduced levels of either or both beta-glucosidase activity and beta-xylosidase activity are required, in the presence of an equivalent panel of other enzymes or accessory components, and under an equivalent set of conditions, to achieve a same level of hydrolysis of a given substrate. As such, less total proteins are required to be made and secreted by a suitable host organism in order to arrive at an enzyme mixture of equal effectiveness when the engineered GH3 enzymes in accordance with the present disclosure are incorporated, as compared an enzyme mixture comprising at least one GH3 beta-xylosidase and another GH3 beta-glucosidase. Along these lines, it is also noted that if the same levels of beta-glucosidase and beta-xylosidase activities are included in an enzyme mixture through the use of the engineered GH3 enzyme herein, that enzyme mixture will have improved biomass hydrolysis performance as compared to a counterpart enzyme mixture achieving the same levels of beta-glucosidase and beta-xylosidase activities through the use of separate GH3 enzymes.

### BRIEF DESCRIPTION OF THE DRAWINGS

FIG. 1 depicts the 3-D crystallographic structure of Trichoderma reesei beta-glucosidase I (Bgl1). Domain 1 is colored in white, domain 2 is colored in gray, and domain 3 is colored in black.

FIG. 2 depicts the 3-D crystallographic structure of Trichoderma reesei beta-xylosidase 3A (Xyl3A). Domain 1 is colored in white, domain 2 is colored in gray, and domain 3 is colored in black.

FIG. 3 compares the active sites of Bgl1 complexed with glucose (in black) and Xyl3A complexed with 4-thioxylobiose (in white). It can be seen that the tryptophan 87 residue of Xyl3A, shown in stick representation, clashes with the C6-group of the glucose.

FIG. 4 is a closeup picture of residues that determine differences in specificity of Bgl1 (in black) and Xyl3A (in white). “TX2” marks the 4-thioxylobiose, whereas “BGC” marks the beta-glucose. Also indicated were the C6 and O6 atoms of beta-glucose that clash with Xyl3A tryptophan 87 residue.

FIG. 5 depicts SDS-PAGE results of the production of T. reesei Bgl1 variants, as following the numbering of those variants according to Table 4. Wild type T. reesei Bgl1 is marked as “wt.”

FIGS. 6A-6E depict activities of variants 2, 3 and/or 12 of T. reesei Bgl1. FIG. 6A depicts beta-xylosidase activity of the variants. FIG. 6B depicts steady state kinetics for hydrolysis of pNpX by Bgl1 variant 03 (Var03), as compared to Bgl1 wild type (“WT”). FIG. 6C depicts steady state kinetics for hydrolysis of pNpX by Xyl3A and Bgl1 variant 03, as compared to Bgl1 wild type. FIG. 6D depicts steady state kinetics for beta-glucosidase activity of Bgl1 variants 02, 03, 12. (Bxl1 indicates T. reesei wild type beta-xylosidase 1 Bxl1.) FIG. 6E depicts steady state kinetics of hydrolysis of pNpG by Bgl1 Var.03 and Bgl1 WT.

FIGS. 7A-7G depict modeled structures of Bgl1 variants 02 (FIGS. 7A & 7B), 03 (FIGS. 7C & 7D), and 12 (FIGS. 7E & 7F) with either glucose (FIGS. 7A, 7C & 7E) or xylose (FIGS. 7B, 7D & 7F) bound in the active site. Bgl1 WT with glucose bound in the active site (pdb 3ZYZ) is shown for comparison (FIG. 7G). Models were constructed using Pymol.

FIG. 8 depicts suitable signal sequences and sequence identifiers of the present disclosure.

### DETAILED DESCRIPTION

Described are certain GH3 beta-glucosidase enzymes that have been engineered or modified to change specificity. The engineered GH3 beta-glucosidase as described herein, in particular embodiments, may have improved beta-glucosidase activity as compared to the parent enzyme, while at the same time also acquire additional substrate specificity to xylosides. As a result, the GH3 beta-glucosidase of the present invention can be modified at certain key residues such that the resulting engineered enzymes will acquire beta-xylosidase activity. For example, the engineered GH3 beta-glucosidase will have not only beta-glucosidase activity but also beta-xylosidase activity. As such, the engineered enzyme has higher beta-xylosidase activity than that of its native, unengineered, parent beta-glucosidase. Also, certain of the GH3 beta-glucosidase of the present invention can be modified at key residues such that the resulting engineered enzymes will acquire beta-xylosidase activity. It is further contemplated that certain of the engineered GH3 beta-glucosidase of the present invention can be modified at key residues in such a way that the resulting engineered enzyme acquires beta-xylosidase activity while retaining substantially all of the beta-glucosidase activity of the parent, or even has increased beta-glucosidase activity as compared to the parent enzymes before it is engineered. On the other hand, contemplated are such engineered GH3 beta-glucosidase enzymes that have lost most (i.e. 50% or more) of its beta-glucosidase activity, and has gained sufficient level of beta-xylosidase activity such that the engineered enzyme can be primarily deemed a beta-xylosidase.

Before the present compositions and methods are described in greater detail, it is to be understood that the present compositions and methods are not limited to particular embodiments described, as such may, of course, vary. It is also to be understood that the terminology used herein is for the purpose of describing particular embodiments only, and is not intended to be limiting, since the scope of the present compositions and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the present compositions and methods. The upper and lower limits of these smaller ranges may independently be included in the smaller ranges and are also encompassed within the present compositions and methods, subject to any specifically excluded limit in the stated range. Where the stated range includes one or both of the limits, ranges excluding either or both of those included limits are also included in the present compositions and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. For example, in connection with a numerical value, the term “about” refers to a range of −10% to +10% of the numerical value, unless the term is otherwise specifically defined in context. In another example, the phrase a “pH value of about 6” refers to pH values of from 5.4 to 6.6, unless the pH value is specifically defined otherwise.

The headings provided herein are not limitations of the various aspects or embodiments of the present compositions and methods which can be had by reference to the specification as a whole. Accordingly, the terms defined immediately below are more fully defined by reference to the specification as a whole.

The present document is organized into a number of sections for ease of reading; however, the reader will appreciate that statements made in one section may apply to other sections. In this manner, the headings used for different sections of the disclosure should not be construed as limiting.

Unless defined otherwise, all technical and scientific terms used herein have the same meaning as commonly understood by one of ordinary skill in the art to which the present compositions and methods belongs. Although any methods and materials similar or equivalent to those described herein can also be used in the practice or testing of the present compositions and methods, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present compositions and methods are not entitled to antedate such publication by virtue of prior invention. Further, the dates of publication provided may be different from the actual publication dates which may need to be independently confirmed.

In accordance with this detailed description, the following abbreviations and definitions apply. Note that the singular forms “a,” “an,” and “the” include plural referents unless the context clearly dictates otherwise. Thus, for example, reference to “an enzyme” includes a plurality of such enzymes, and reference to “the dosage” includes reference to one or more dosages and equivalents thereof known to those skilled in the art, and so forth.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The term “engineered,” when used in reference to a subject cell, nucleic acid, polypeptides/enzymes or vector, indicates that the subject has been modified from its native state. Thus, for example, engineered cells express genes that are not found within the native (non-recombinant) form of the cell, or express native genes at different levels or under different conditions than found in nature. Engineered nucleic acids may differ from a native sequence by one or more nucleotides and/or are operably linked to heterologous sequences, e.g., a heterologous promoter, signal sequences that allow secretion, etc., in an expression vector. Engineered polypeptides/enzymes may differ from a native sequence by one or more amino acids and/or are fused with heterologous sequences. A vector comprising a nucleic acid encoding an engineered GH3 enzyme as described herein is, for example, an engineered vector. The term “engineered” can be used interchangeably as the term “recombinant” herein.

It is further noted that the term “consisting essentially of,” as used herein refers to a composition wherein the component(s) after the term is in the presence of other known component(s) in a total amount that is less than 30% by weight of the total composition and do not contribute to or interferes with the actions or activities of the component(s).

It is further noted that the term “comprising,” as used herein, means including, but not limited to, the component(s) after the term “comprising.” The component(s) after the term “comprising” are required or mandatory, but the composition comprising the component(s) may further include other non-mandatory or optional component(s).

It is also noted that the term “consisting of,” as used herein, means including, and limited to, the component(s) after the term “consisting of.” The component(s) after the term “consisting of” are therefore required or mandatory, and no other component(s) are present in the composition.

As will be apparent to those of skill in the art upon reading this disclosure, each of the individual embodiments described and illustrated herein has discrete components and features which may be readily separated from or combined with the features of any of the other several embodiments without departing from the scope or spirit of the present compositions and methods described herein. Any recited method can be carried out in the order of events recited or in any other order which is logically possible.

“Beta-glucosidase” refers to a beta-D-glucoside glucohydrolase of E.C. 3.2.1.21. The term “beta-glucosidase activity” therefore refers the capacity of catalyzing the hydrolysis of beta-D-glucoside, such as cellobiose to release D-glucose. Beta-glucosidase activity may be determined using a cellobiase assay, for example, which measures the capacity of the enzyme to catalyze the hydrolysis of a cellobiose substrate to yield D-glucose. Furthermore, beta-glucosidase activity can also be determined using model substrates such as pNpG, as described herein.

As used herein, the term “β-xylosidase” refers to a beta-D-glucoside glucohydrolase of E.C. 3.2.1.37. The term “beta-xylosidase” activity” therefore refers to the capacity of catalyzing the hydrolysis of beta-D-xylosides, such as xylobiose, or para-nitro-phenol-beta-D-xylose (pNpX) to release D-xylose. Beta-xylosidase activity may be determined using a xylobiase assay, for example, which measures the capacity of the enzyme to catalyze the hydrolysis of a xylobiose substrate to yield D-xylose. Suitable β-xylosidases include, for example Talaromyces emersonii Bxl1 (Reen et al., 2003, Biochem. Biophys. Res. Commun. 305(3):579-85); as well as β-xylosidases obtained from Geobacillus stearothermophilus (Shallom et al., 2005, Biochem. 44:387-397); Scytalidium thermophilum (Zanoelo et al., 2004, J. Ind. Microbiol. Biotechnol. 31:170-176); Trichoderma lignorum (Schmidt, 1988, Methods Enzymol. 160:662-671); Aspergillus awamori (Kurakake et al., 2005, Biochim. Biophys. Acta 1726:272-279); Aspergillus versicolor (Andrade et al., Process Biochem. 39:1931-1938); Streptomyces sp. (Pinphanichakarn et al., 2004, World J. Microbiol. Biotechnol. 20:727-733); Thermotoga maritima (Xue and Shao, 2004, Biotechnol. Lett. 26:1511-1515); Trichoderma sp. SY (Kim et al., 2004, J. Microbiol. Biotechnol. 14:643-645); Aspergillus niger (Oguntimein and Reilly, 1980, Biotechnol. Bioeng. 22:1143-1154); or Penicillium wortmanni (Matsuo et al., 1987, Agric. Biol. Chem. 51:2367-2379).

In certain aspects, the β-xylosidase does not have retaining β-xylosidase activity. In other aspects, the β-xylosidase has inverting β-xylosidase activity. In yet further aspects, the β-xylosidase has no retaining β-xylosidase activity but has inverting β-xylosidase activity. An enzyme can be tested for retaining vs. inverting activity. Generally cleavage of a glycosidic bond by b-xylosidases has been shown to follow either of the two mechanisms, the stereochemical outcome of which is an overall retention (i.e., the retaining mechanism or the “retaining b-xylosidase activity”) or inversion (i.e., the inverting mechanism or the “inverting b-xylosidase activity”) of the configuration of aromeric center of glycon part of substrate. M. Sinnott, Chem. Rev., 90:1170-1202 (1990); J. McCarter & S. Withers, Curr. Opin. Struct. Biol. 4:885-892 (1994).

“Family 3 glycosyl hydrolase” or “GH3” refers to polypeptides falling within the definition of glycosyl hydrolase family 3 according to the classification by Henrissat, Biochem. J. 280:309-316 (1991), and by Henrissat & Cairoch, Biochem. J., 316:695-696 (1996).

An engineered GH3 enzyme, according to the present compositions and methods described herein, can be isolated or purified. By purification or isolation is meant that the GH3 polypeptide is altered from its natural state by the simple fact that the molecule and the amino acid sequence of it does not exist in nature, or by virtue of separating the GH3 from some or all of the naturally occurring constituents with which it is associated in nature. Isolation or purification may be accomplished by art-recognized separation techniques such as ion exchange chromatography, affinity chromatography, hydrophobic separation, dialysis, protease treatment, ammonium sulphate precipitation or other protein salt precipitation, centrifugation, size exclusion chromatography, filtration, microfiltration, gel electrophoresis or separation on a gradient to remove whole cells, cell debris, impurities, extraneous proteins, or enzymes undesired in the final composition. It is further possible to then add constituents to the engineered GH3 enzyme-containing composition which provide additional benefits, for example, activating agents, anti-inhibition agents, desirable ions, compounds to control pH or other enzymes or chemicals.

As used herein, “microorganism” refers to a bacterium, a fungus, a virus, a protozoan, and other microbes or microscopic organisms.

As used herein, a “derivative” or “variant” of a polypeptide means a polypeptide, which is derived from a precursor polypeptide (e.g., the native polypeptide or the parent GH3 polypeptide) by addition of one or more amino acids to either or both the C- and N-terminal end, substitution of one or more amino acids at one or a number of different sites in the amino acid sequence, deletion of one or more amino acids at either or both ends of the polypeptide or at one or more sites in the amino acid sequence, or insertion of one or more amino acids at one or more sites in the amino acid sequence. The preparation of a GH3 polypeptide derivative or variant may be achieved in any convenient manner, e.g., by modifying a DNA sequence which encodes the native or parent polypeptides, transformation of that DNA sequence into a suitable host, and expression of the modified DNA sequence to form the derivative/variant GH3 enzyme. Derivatives or variants further include GH3 polypeptides that are chemically modified, e.g., glycosylation or otherwise changing a characteristic of the parent GH3 polypeptide. While derivatives and variants of GH3 polypeptides are encompassed by the present compositions and methods, such derivates and variants will at times display dual functionality, for example, in the case of a parent GH3 beta-glucosidase, acquiring beta-xylosidase activity without completely losing beta-glucosidase activity (i.e., retaining at least some beta-glucosidase activity), or in the case of a parent GH3 beta-xylosidase, acquiring beta-glucosidase activity without completely losing beta-xylosidase activity (i.e., retaining at least some beta-xylosidase activity). In certain specific embodiments, a parent GH3 beta-glucosidase, having been engineered to acquire beta-xylosidase activity while retaining substantially all of its parent's beta-glucosidase activity, the resulting engineered enzyme is deemed a variant or a derivative of the parent GH3 polypeptide hereunder. In particular embodiments, a parent GH3 beta-glucosidase, having been engineered to acquire beta-xylosidase activity but at the same time acquire improved or increased beta-glucosidase even when compared to the beta-glucosidase activity of the parent, and such a resulting engineered enzyme is also deemed a variant or a derivative of the parent GH3 polypeptide.

As used herein, “percent (%) sequence identity” with respect to the amino acid or nucleotide sequences identified herein is defined as the percentage of amino acid residues or nucleotides in a candidate sequence that are identical with the amino acid residues or nucleotides in a parent GH3 enzyme sequence, after aligning the sequences and introducing gaps, if necessary, to achieve the maximum percent sequence identity, and not considering any conservative substitutions as part of the sequence identity.

By “homologue” shall mean an entity having a specified degree of identity with the subject amino acid sequences and the subject nucleotide sequences. A homologous sequence is taken to include an amino acid sequence that is at least 70%, 75%, 80%, 85%, 86%, 87%, 88%, 89%, 90%, 91%, 92%, 93%, 94%, 95%, 96%, 97%, 98% or even 99% identical to the subject sequence, using conventional sequence alignment tools (e.g., Clustal, BLAST, and the like). Typically, homologues will include the same active site residues as the subject amino acid sequence, unless otherwise specified.

Methods for performing sequence alignment and determining sequence identity are known to the skilled artisan, may be performed without undue experimentation, and calculations of identity values may be obtained with definiteness. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Protein Sequence and Structure 5:Suppl. 3 (National Biomedical Research Foundation, Washington, D.C.). A number of algorithms are available for aligning sequences and determining sequence identity and include, for example, the homology alignment algorithm of Needleman et al. (1970) J. Mol. Biol. 48:443; the local homology algorithm of Smith et al. (1981) Adv. Appl. Math. 2:482; the search for similarity method of Pearson et al. (1988) Proc. Natl. Acad. Sci. 85:2444; the Smith-Waterman algorithm (Meth. Mol. Biol. 70:173-187 (1997); and BLASTP, BLASTN, and BLASTX algorithms (see Altschul et al. (1990) J. Mol. Biol. 215:403-410).

Computerized programs using these algorithms are also available, and include, but are not limited to: ALIGN or Megalign (DNASTAR) software, or WU-BLAST-2 (Altschul et al., (1996) Meth. Enzym., 266:460-480); or GAP, BESTFIT, BLAST, FASTA, and TFASTA, available in the Genetics Computing Group (GCG) package, Version 8, Madison, Wis., USA; and CLUSTAL in the PC/Gene program by Intelligenetics, Mountain View, Calif. Those skilled in the art can determine appropriate parameters for measuring alignment, including algorithms needed to achieve maximal alignment over the length of the sequences being compared. Preferably, the sequence identity is determined using the default parameters determined by the program. Specifically, sequence identity can determined by using Clustal W (Thompson J. D. et al. (1994) Nucleic Acids Res. 22:4673-4680) with default parameters, i.e.:
Gap opening penalty: 10.0
Gap extension penalty: 0.05
Protein weight matrix: BLOSUM series
DNA weight matrix: TUB
Delay divergent sequences %: 40
Gap separation distance: 8
DNA transitions weight: 0.50
List hydrophilic residues: GPSNDQEKR
Use negative matrix: OFF
Toggle Residue specific penalties: ON
Toggle hydrophilic penalties: ON
Toggle end gap separation penalty OFF

As used herein, “expression vector” means a DNA construct including a DNA sequence which is operably linked to a suitable control sequence capable of affecting the expression of the DNA in a suitable host. Such control sequences may include a promoter to affect transcription, an optional operator sequence to control transcription, a sequence encoding suitable ribosome-binding sites on the mRNA, and sequences which control termination of transcription and translation. Different cell types may be used with different expression vectors. An exemplary promoter for vectors used in Bacillus subtilis is the AprE promoter; an exemplary promoter used in Streptomyces lividans is the A4 promoter (from Aspergillus niger); an exemplary promoter used in E. coli is the Lac promoter, an exemplary promoter used in Saccharomyces cerevisiae is PGK1, an exemplary promoter used in Aspergillus niger is glaA, and an exemplary promoter for Trichoderma reesei is cbh1. The vector may be a plasmid, a phage particle, or simply a potential genomic insert. Once transformed into a suitable host, the vector may replicate and function independently of the host genome, or may, under suitable conditions, integrate into the genome itself. In the present specification, plasmid and vector are sometimes used interchangeably. However, the present compositions and methods are intended to include other forms of expression vectors which serve equivalent functions and which are, or become, known in the art. Thus, a wide variety of host/expression vector combinations may be employed in expressing the DNA sequences described herein. Useful expression vectors, for example, may consist of segments of chromosomal, non-chromosomal and synthetic DNA sequences such as various known derivatives of SV40 and known bacterial plasmids, e.g., plasmids from E. coli including col E1, pCR1, pBR322, pMb9, pUC 19 and their derivatives, wider host range plasmids, e.g., RP4, phage DNAs e.g., the numerous derivatives of phage λ, e.g., NM989, and other DNA phages, e.g., M13 and filamentous single stranded DNA phages, yeast plasmids such as the 2μ plasmid or derivatives thereof, vectors useful in eukaryotic cells, such as vectors useful in animal cells and vectors derived from combinations of plasmids and phage DNAs, such as plasmids which have been modified to employ phage DNA or other expression control sequences. Expression techniques using the expression vectors of the present compositions and methods are known in the art and are described generally in, for example, Sambrook et al., Molecular Cloning: A Laboratory Manual, Second Edition, Cold Spring Harbor Press (1989). Often, such expression vectors including the DNA sequences described herein are transformed into a unicellular host by direct insertion into the genome of a particular species through an integration event (see e.g., Bennett & Lasure, More Gene Manipulations in Fungi, Academic Press, San Diego, pp. 70-76 (1991) and articles cited therein describing targeted genomic insertion in fungal hosts).

As used herein, “host strain” or “host cell” means a suitable host for an expression vector including DNA according to the present compositions and methods. Host cells useful in the present compositions and methods are generally prokaryotic or eukaryotic hosts, including any transformable microorganism in which expression can be achieved. Specifically, host strains may be Bacillus subtilis, Bacillus hemicellulosilyticus, Streptomyces lividans, Escherichia coli, Trichoderma reesei, Saccharomyces cerevisiae, Aspergillus niger, Aspergillus oryzae, Chrysosporium lucknowence, Myceliophthora thermophila, and various other microbial cells. Host cells are transformed or transfected with vectors constructed using recombinant DNA techniques. Such transformed host cells may be capable of one or both of replicating the vectors encoding a GH3 enzyme (and its derivatives or variants (mutants) and expressing the desired peptide product. In certain embodiments according to the present compositions and methods, wherein “host cell” is used in reference to Trichoderma sp., it means both the cells and protoplasts created from the cells of Trichoderma sp.

A “host strain” or “host cell” is an organism into which an expression vector, phage, virus, or other DNA construct, including a polynucleotide encoding a polypeptide of interest (e.g., an engineered GH3 enzyme) has been introduced. Exemplary host strains are microbial cells (e.g., bacteria, filamentous fungi, and yeast) capable of expressing the polypeptide of interest. The term “host cell” includes protoplasts created from cells.

The terms “transformed,” “stably transformed,” and “transgenic,” used with reference to a cell means that the cell contains a non-native (e.g., heterologous) nucleic acid sequence integrated into its genome or carried as an episome that is maintained through multiple generations.

The term “introduced” in the context of inserting a nucleic acid sequence into a cell, means “transfection”, “transformation” or “transduction,” as known in the art. Means of transformation include protoplast transformation, calcium chloride precipitation, electroporation, naked DNA, and the like as known in the art. (See, Chang and Cohen (1979) Mol. Gen. Genet. 168:111-115; Smith et al., (1986) Appl. Env. Microbiol. 51:634; and the review article by Ferrari et al., in Harwood, Bacillus, Plenum Publishing Corporation, pp. 57-72, 1989).

The term “heterologous” with reference to a polynucleotide or polypeptide refers to a polynucleotide or polypeptide that does not naturally occur in a host cell.

The term “endogenous” with reference to a polynucleotide or polypeptide refers to a polynucleotide or polypeptide that occurs naturally in the host cell.

The term “expression” refers to the process by which a polypeptide is produced based on a nucleic acid sequence. The process includes both transcription and translation.

As used herein, “signal sequence” means a sequence of amino acids bound to the N-terminal portion of a protein which facilitates the secretion of the mature form of the protein outside of the cell. This definition of a signal sequence is a functional one. The mature form of the extracellular protein lacks the signal sequence which is cleaved off during the secretion process. While the native signal sequence of parent GH3 beta-glucosidase or GH3 beta-xylosidase may be employed in aspects of the present compositions and methods, other non-native signal sequences may also be employed (e.g., one selected from SEQ ID NOs:8-36).

The engineered GH3 polypeptides of the invention may be referred to as “precursor,” “immature,” or “full-length,” in which case they include a signal sequence, or may be referred to as “mature,” in which case they lack a signal sequence. Mature forms of the polypeptides are generally the most useful. Unless otherwise noted, the amino acid residue numbering used herein refers to the mature forms of the respective GH3 polypeptides. The engineered GH3 polypeptides of the invention may also be truncated to remove the N or C-termini, so long as the resulting polypeptides retain the desired beta-glucosidase and/or beta-xylosidase activity.

The engineered GH3 polypeptides of the invention may also be a “chimeric” or “hybrid” polypeptide, in that it includes at least a portion of a first GH3 polypeptide, and at least a portion of a second GH3 polypeptide (such chimeric GH3 polypeptides may, for example, be derived from the first and second GH3 polypeptides using known technologies involving the swapping of domains on each of the GH3 polypeptides). The present engineered GH3 polypeptides may further include heterologous signal sequence, an epitope to allow tracking or purification, or the like. When the term of “heterologous” is used to refer to a signal sequence used to express a polypeptide of interest, it is meant that the signal sequence is, for example, derived from a different microorganism as the polypeptide of interest. Examples of suitable heterologous signal sequences for expressing the engineered GH3 polypeptides herein, may be, for example, those from Trichoderma reesei, other Trichoderma sp., Aspergillus niger, Aspergillus oryzae, other Aspergillus sp., Chrysosporium, and other organisms, those from Bacillus subtilis, Bacillus hemicellulosilyticus, other Bacillus species, E. coli., or other suitable microbes.

As used herein, “functionally attached” or “operably linked” means that a regulatory region or functional domain having a known or desired activity, such as a promoter, terminator, signal sequence or enhancer region, is attached to or linked to a target (e.g., a gene or polypeptide) in such a manner as to allow the regulatory region or functional domain to control the expression, secretion or function of that target according to its known or desired activity.

As used herein, the terms “polypeptide” and “enzyme” are used interchangeably to refer to polymers of any length comprising amino acid residues linked by peptide bonds. The conventional one-letter or three-letter codes for amino acid residues are used herein. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, “wild-type” and “native” genes, enzymes, or strains, are those found in nature.

The terms “wild-type,” “parent,” “parental” or “reference,” with respect to a polypeptide, refer to a naturally-occurring polypeptide that does not include a man-made substitution, insertion, or deletion at one or more amino acid positions. Similarly, the term “wild-type,” “parent,” “parental,” or “reference,” with respect to a polynucleotide, refers to a naturally-occurring polynucleotide that does not include a man-made nucleoside change. However, a polynucleotide encoding a wild-type, parental, or reference polypeptide is not limited to a naturally-occurring polynucleotide, but rather encompasses any polynucleotide encoding the wild-type, parental, or reference polypeptide.

As used herein, a “variant polypeptide” refers to a polypeptide that is derived from a parent (or reference) polypeptide by the substitution, addition, or deletion, of one or more amino acids, typically by recombinant DNA techniques. Variant polypeptides may differ from a parent polypeptide by a small number of amino acid residues. They may be defined by their level of primary amino acid sequence homology/identity with a parent polypeptide. Suitably, variant polypeptides have at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity to a parent polypeptide.

As used herein, a “variant polynucleotide” encodes a variant polypeptide, has a specified degree of homology/identity with a parent polynucleotide, or hybridized under stringent conditions to a parent polynucleotide or the complement thereof. Suitably, a variant polynucleotide has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity to a parent polynucleotide or to a complement of the parent polynucleotide. Methods for determining percent identity are known in the art and described above.

The term “derived from” encompasses the terms “originated from,” “obtained from,” “obtainable from,” “isolated from,” and “created from,” and generally indicates that one specified material find its origin in another specified material or has features that can be described with reference to the another specified material.

As used herein, the term “hybridization conditions” refers to the conditions under which hybridization reactions are conducted. These conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm −5° C. (5° C. below the Tm of the probe); “high stringency” at about 5-10° C. below the Tm; “intermediate stringency” at about 10-20° C. below the Tm of the probe; and “low stringency” at about 20-25° C. below the Tm. Alternatively, or in addition, hybridization conditions can be based upon the salt or ionic strength conditions of hybridization, and/or upon one or more stringency washes, e.g.: 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, it is typically desirable to use relatively stringent conditions to form the hybrids (e.g., relatively low salt and/or high temperature conditions are used).

As used herein, the term “hybridization” refers to the process by which a strand of nucleic acid joins with a complementary strand through base pairing, as known in the art. More specifically, “hybridization” refers to the process by which one strand of nucleic acid forms a duplex with, i.e., base pairs with, a complementary strand, as occurs during blot hybridization techniques and PCR techniques. A nucleic acid sequence is considered to be “selectively hybridizable” to a reference nucleic acid sequence if the two sequences specifically hybridize to one another under moderate to high stringency hybridization and wash conditions. Hybridization conditions are based on the melting temperature (Tm) of the nucleic acid binding complex or probe. For example, “maximum stringency” typically occurs at about Tm-5° C. (5° below the Tm of the probe); “high stringency” at about 5-10° C. below the Tm; “intermediate stringency” at about 10-20° C. below the Tm of the probe; and “low stringency” at about 20-25° C. below the Tm. Functionally, maximum stringency conditions may be used to identify sequences having strict identity or near-strict identity with the hybridization probe; while intermediate or low stringency hybridization can be used to identify or detect polynucleotide sequence homologs.

Intermediate and high stringency hybridization conditions are well known in the art. For example, intermediate stringency hybridizations may be carried out with an overnight incubation at 37° C. in a solution comprising 20% formamide, 5×SSC (150 mM NaCl, 15 mM trisodium citrate), 50 mM sodium phosphate (pH 7.6), 5×Denhardt's solution, 10% dextran sulfate and 20 mg/ml denatured sheared salmon sperm DNA, followed by washing the filters in 1×SSC at about 37-50° C. High stringency hybridization conditions may be hybridization at 65° C. and 0.1×SSC (where 1×SSC=0.15 M NaCl, 0.015 M Na₃ citrate, pH 7.0). Alternatively, high stringency hybridization conditions can be carried out at about 42° C. in 50% formamide, 5×SSC, 5×Denhardt's solution, 0.5% SDS and 100 μg/ml denatured carrier DNA followed by washing two times in 2×SSC and 0.5% SDS at room temperature and two additional times in 0.1×SSC and 0.5% SDS at 42° C. And very high stringent hybridization conditions may be hybridization at 68° C. and 0.1×SSC. Those of skill in the art know how to adjust the temperature, ionic strength, etc. as necessary to accommodate factors such as probe length and the like.

A nucleic acid encoding a variant beta-xylosidase, or an engineered multi-functional GH3 enzyme may have a Tₘ increased, or reduced by 1° C.-3° C. or more compared to a duplex formed between the nucleotide of SEQ ID NO: 1, or SEQ ID NO:4, and its identical complement.

The phrase “substantially similar” or “substantially identical,” in the context of at least two nucleic acids or polypeptides, means that a polynucleotide or polypeptide comprises a sequence that has at least about 90%, at least about 91%, at least about 92%, at least about 93%, at least about 94%, at least about 95%, at least about 96%, at least about 97%, at least about 98%, or even at least about 99% identical to a parent or reference sequence, or does not include amino acid substitutions, insertions, deletions, or modifications made only to circumvent the present description without adding functionality.

As used herein, an “expression vector” refers to a DNA construct containing a DNA sequence that encodes a specified polypeptide and is operably linked to a suitable control sequence capable of effecting the expression of the polypeptides in a suitable host. Such control sequences may include a promoter to effect transcription, an optional operator sequence to control such transcription, a sequence encoding suitable mRNA ribosome binding sites and/or sequences that control termination of transcription and translation. The vector may be a plasmid, a phage particle, or a potential genomic insert. Once transformed into a suitable host, the vector may replicate and function independently of the host genome, or may, in some instances, integrate into the host genome.

The term “selective marker” or “selectable marker,” refers to a gene capable of expression in a host cell that allows for ease of selection of those hosts containing an introduced nucleic acid or vector. Examples of selectable markers include but are not limited to antimicrobial substances (e.g., hygromycin, bleomycin, or chloramphenicol) and/or genes that confer a metabolic advantage, such as a nutritional advantage, on the host cell.

The term “regulatory element” or “regulatory sequence” refers to a genetic element that controls some aspect of the expression of nucleic acid sequences. For example, a promoter is a regulatory element which facilitates the initiation of transcription of an operably linked coding region. Additional regulatory elements include splicing signals, polyadenylation signals and termination signals.

As used herein, “host cells” are generally cells of prokaryotic or eukaryotic hosts that are transformed or transfected with vectors constructed using recombinant DNA techniques known in the art. Transformed host cells are capable of either replicating vectors encoding the polypeptide variants or expressing the desired polypeptide variant. In the case of vectors, which encode the pre- or pro-form of the polypeptide variant, such variants, when expressed, are typically secreted from the host cell into the host cell medium.

The term “introduced,” in the context of inserting a nucleic acid sequence into a cell, means transformation, transduction, or transfection. Means of transformation include protoplast transformation, calcium chloride precipitation, electroporation, naked DNA, and the like as known in the art. (See, Chang and Cohen (1979) Mol. Gen. Genet. 168:111-115; Smith et al., (1986) Appl. Env. Microbiol. 51:634; and the review article by Ferrari et al., in Harwood, Bacillus, Plenum Publishing Corporation, pp. 57-72, 1989).

“Fused” polypeptide sequences are connected, i.e., operably linked, via a peptide bond between two subject polypeptide sequences.

The term “filamentous fungi” refers to all filamentous forms of the subdivision Eumycotina, particularly Pezizomycotina species.

Other technical and scientific terms have the same meaning as commonly understood by one of ordinary skill in the art to which this disclosure pertains (See, e.g., Singleton and Sainsbury, Dictionary of Microbiology and Molecular Biology, 2d Ed., John Wiley and Sons, NY 1994; and Hale and Marham, The Harper Collins Dictionary of Biology, Harper Perennial, NY 1991).

The Trichoderma reesei beta-glucosidase 1 (Bgl1) (SEQ ID NO:2) has the following amino acid sequence, with the predicted signal sequence (as per SignalP 4.1, available at http://www.cbs.dtu.dk/cgi-bin/webface2.fcgi?jobid=545F0CC500006119218EDD7C&wait=20) underlined:

MRYRTAAALALATGPFARADSHSTSGASAEAVVPPAGTPWGTAYDKAKA
ALAKLNLQDKVGIVSGVGWNGGPCVGNTSPASKISYPSLCLQDGPLGVR
YSTGSTAFTPGVQAASTWDVNLIRERGQFIGEEVKASGIHVILGPVAGP
LGKTPQGGRNWEGFGVDPYLTGIAMGQTINGIQSVGVQATAKHYILNEQ
ELNRETISSNPDDRTLHELYTWPFADAVQANVASVMCSYNKVNTTWACE
DQYTLQTVLKDQLGFPGYVMTDWNAQHTTVQSANSGLDMSMPGTDFNGN
NRLWGPALTNAVNSNQVPTSRVDDMVTRILAAWYLTGQDQAGYPSFNIS
RNVQGNHKTNVRAIARDGIVLLKNDANILPLKKPASIAVVGSAAIIGNH
ARNSPSCNDKGCDDGALGMGWGSGAVNYPYFVAPYDAINTRASSQGTQV
TLSNTDNTSSGASAARGKDVAIVFITADSGEGYITVEGNAGDRNNLDPW
HNGNALVQAVAGANSNVIVVVHSVGAIILEQILALPQVKAVVWAGLPSQ
ESGNALVDVLWGDVSPSGKLVYTIAKSPNDYNTRIVSGGSDSFSEGLFI
DYKHFDDANITPRYEFGYGLSYTKFNYSRLSVLSTAKSGPATGAVVPGG
PSDLFQNVATVTVDIANSGQVTGAEVAQLYITYPSSAPRTPPKQLRGFA
KLNLTPGQSGTATFNIRRRDLSYWDTASQKWVVPSGSFGISVGASSRDI
RLTSTLSVA

The mature Trichoderma reesei Bgl1 enzyme, as based on the removal of the predicted signal peptide sequence is SEQ ID NO:3:

VVPPAGTPWGTAYDKAKAALAKLNLQDKVGIVSGVGWNGGPCVGNTSPA
SKISYPSLCLQDGPLGVRYSTGSTAFTPGVQAASTWDVNLIRERGQFIG
EEVKASGIHVILGPVAGPLGKTPQGGRNWEGFGVDPYLTGIAMGQTING
IQSVGVQATAKHYILNEQELNRETISSNPDDRTLHELYTWPFADAVQAN
VASVMCSYNKVNTTWACEDQYTLQTVLKDQLGFPGYVMTDWNAQHTTVQ
SANSGLDMSMPGTDFNGNNRLWGPALTNAVNSNQVPTSRVDDMVTRILA
AWYLTGQDQAGYPSFNISRNVQGNHKTNVRAIARDGIVLLKNDANILPL
KKPASIAVVGSAAIIGNHARNSPSCNDKGCDDGALGMGWGSGAVNYPYF
VAPYDAINTRASSQGTQVTLSNTDNTSSGASAARGKDVAIVFITADSGE
GYITVEGNAGDRNNLDPWHNGNALVQAVAGANSNVIVVVHSVGAIILEQ
ILALPQVKAVVWAGLPSQESGNALVDVLWGDVSPSGKLVYTIAKSPNDY
NTRIVSGGSDSFSEGLFIDYKHFDDANITPRYEFGYGLSYTKFNYSRLS
VLSTAKSGPATGAVVPGGPSDLFQNVATVTVDIANSGQVTGAEVAQLYI
TYPSSAPRTPPKQLRGFAKLNLTPGQSGTATFNIRRRDLSYWDTASQKW
VVPSGSFGISVGASSRDIRLTSTLSVA

The Trichoderma reesei beta-xylosidase 3 (Xyl3A) (SEQ ID NO:4) has the following amino acid sequence, with the signal sequence underlined:

MVNNAALLAALSALLPTALAQNNQTYANYSAQGQPDLYPETLATLTLSF
PDCEHGPLKNNLVCDSSAGYVERAQALISLFTLEELILNTQNSGPGVPR
LGLPNYQVWNEALHGLDRANFATKGGQFEWATSFPMPILTTAALNRTLI
HQIADIISTQARAFSNSGRYGLDVYAPNVNGFRSPLWGRGQETPGEDAF
FLSSAYTYEYITGIQGGVDPEHLKVAATVKHFAGYDLENWNNQSRLGFD
AIITQQDLSEYYTPQFLAAARYAKSRSLMCAYNSVNGVPSCANSFFLQT
LLRESWGFPEWGYVSSDCDAVYNVFNPHDYASNQSSAAASSLRAGTDID
CGQTYPWHLNESFVAGEVSRGEIERSVTRLYANLVRLGYFDKKNQYRSL
GWKDVVKTDAWNISYEAAVEGIVLLKNDGTLPLSKKVRSIALIGPWANA
TTQMQGNYYGPAPYLISPLEAAKKAGYHVNFELGTEIAGNSTTGFAKAI
AAAKKSDAIIYLGGIDNTIEQEGADRTDIAWPGNQLDLIKQLSEVGKPL
VVLQMGGGQVDSSSLKSNKKVNSLVWGGYPGQSGGVALFDILSGKRAPA
GRLVTTQYPAEYVHQFPQNDMNLRPDGKSNPGQTYIWYTGKPVYEFGSG
LFYTTFKETLASHPKSLKFNTSSILSAPHPGYTYSEQIPVFTFEANIKN
SGKTESPYTAMLFVRTSNAGPAPYPNKWLVGFDRLADIKPGHSSKLSIP
IPVSALARVDSHGNRIVYPGKYELALNTDESVKLEFELVGEEVTIENWP
LEEQQIKDATPDA

The mature Trichoderma reesei Xyl3A enzyme, as based on the removal of the predicted signal peptide sequence is SEQ ID NO:5.

QNNQTYANYSAQGQPDLYPETLATLTLSFPDCEHGPLKNNLVCDSSAGY
VERAQALISLFTLEELILNTQNSGPGVPRLGLPNYQVWNEALHGLDRAN
FATKGGQFEWATSFPMPILTTAALNRTLIHQIADIISTQARAFSNSGRY
GLDVYAPNVNGFRSPLWGRGQETPGEDAFFLSSAYTYEYITGIQGGVDP
EHLKVAATVKHFAGYDLENWNNQSRLGFDAIITQQDLSEYYTPQFLAAA
RYAKSRSLMCAYNSVNGVPSCANSFFLQTLLRESWGFPEWGYVSSDCDA
VYNVFNPHDYASNQSSAAASSLRAGTDIDCGQTYPWHLNESFVAGEVSR
GEIERSVTRLYANLVRLGYFDKKNQYRSLGWKDVVKTDAWNISYEAAVE
GIVLLKNDGTLPLSKKVRSIALIGPWANATTQMQGNYYGPAPYLISPLE
AAKKAGYHVNFELGTEIAGNSTTGFAKAIAAAKKSDAIIYLGGIDNTIE
QEGADRTDIAWPGNQLDLIKQLSEVGKPLVVLQMGGGQVDSSSLKSNKK
VNSLVWGGYPGQSGGVALFDILSGKRAPAGRLVTTQYPAEYVHQFPQND
MNLRPDGKSNPGQTYIWYTGKPVYEFGSGLFYTTFKETLASHPKSLKFN
TSSILSAPHPGYTYSEQIPVFTFEANIKNSGKTESPYTAMLFVRTSNAG
PAPYPNKWLVGFDRLADIKPGHSSKLSIPIPVSALARVDSHGNRIVYPG
KYELALNTDESVKLEFELVGEEVTIENWPLEEQQIKDATPDA

### Engineering GH3 Polypeptides

From structural studies of the substrate binding site of a representative GH3 beta-glucosidase (namely the Trichoderma reesei Bgl1) and the substrate binding site of a representative GH3 beta-xylosidase (namely the Trichoderma reesei Xyl3A), and especially the study of 3-D structure using X-ray crystallography the substrate bound versions of these enzymes, it is discovered that by changing certain residues at the respective substrate binding sites of these GH3 enzymes it would be possible to switch the substrate specificity and enzymatic activities of these enzymes. More specifically, the Xyl3A 3-D crystallographic structure complexed with 4-thioxylobiose at the active site was compared to the Bgl1 3-D crystallographic structure complexed with a glucose at the active site. Superimposing the glucose molecule to the Xyl3A active site allowed the identification of certain active site interactions that would allow 4-thioxylobiose but not a glucose to be substrate to a beta-xylosidase. Conversely, superimposing the 4-thioxylobiose molecule to the Bgl1 active site allowed the identification of active site interactions that would allow/prefer glucose but not a 4-thioxylobiose to be a substrate. Amino acid substitutions at those active sites can then be designed to enable xylosaccharide binding in a GH3 beta-glucosidase and glucosaccharide binding in a GH3 beta-xylosidase.

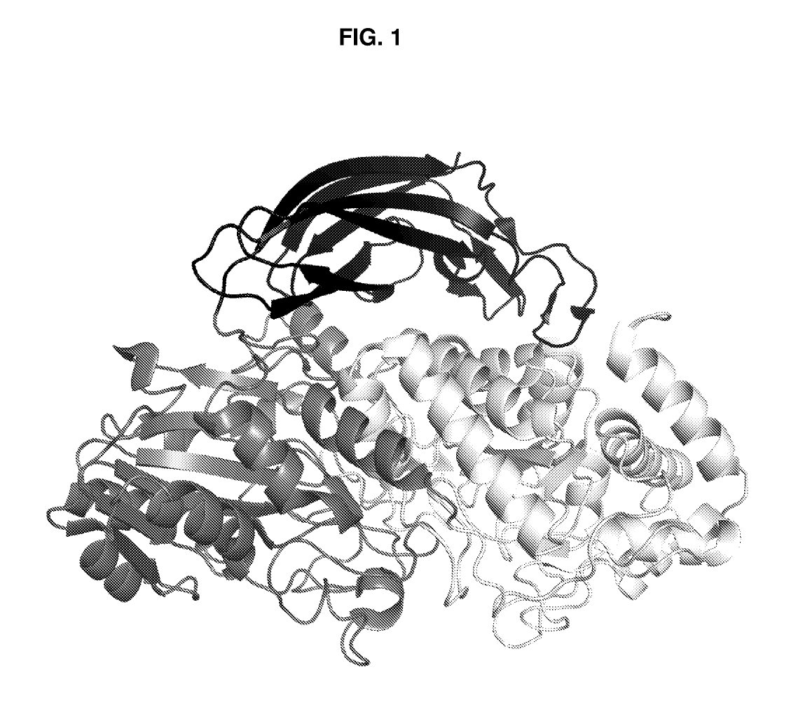

Trichoderma reesei Bgl1 was crystallized with one molecule in the asymmetric unit in space group P2₁, both apo (Bgl1-apo), glucose (Bgl1-glucose) forms, and these structures were solved to a resolution of 2.1 Å. It was noted that the overall structure or “fold” of Trichoderma reesei Bgl1 looks very much like the structure of Thermotoga neapolitana beta-glucosidase 3B. See, Pozzo, T., et al., (2010) Structural and Functional Analysis of Beta-Glucosidase 3B from Thermotoga neapolitana: A Thermostable Three-Domain Representative of Glycosyl Hydrolase 3, J. Mol. Biol., 397:724-739. There are three distinct domains (as seen in FIG. 1). In fact, superimposing the Trichoderma reesei Bgl1 structure with the Thermotoga neapolitana Bgl3B structure gives a root-mean-square deviation (RMSD) of 1.63 Å for 713 equivalent Cα positions, using the SSM algorithm, which is described in Krissinel, E., and Henrick, K., (2004) Secondary-structure Matching (SSM), a New Tool for Fast Protein Structure Alignment in Three Dimensions, Acta Crysallogr. D. Biol. Crysallogr. 60:2256-68.

It can be observed that domain 1 encompasses residues 7 to 300 of Trichodema reesei Bgl1. Domain 1 is joined to domain 2 with a 16-residue linker (i.e., residues 301 to 316). Domain 2, which is a five-stranded α/β sandwich, includes residues 317 to 522. This domain is followed by a domain 3 including residues 580 to 714. It is noted that domain 3 may have an immunoglobulin-like topology. The first two domains are similar to those present in the structure of a GH3 glycosyl hydrolyase obtained from the grain barley. See, Varghese, J. N., et al., (1999) Three-dimensional Structure of a Barley Beta-D-Glucan Exohydrolase, a Family 3 Glycosyl Hydrolase, Structure 7(2):179-90. What differentiates the Barley beta-D-glucan exohydrolase is a canonical TIM barrel fold with an alternating repeat of 8 α-helices and eight parallel β-strands α/β barrel in domain 1, as compared to the T. reesei Bgl1 lacking 3 of the 8 parallel β-strands and the two intervening α-helices. Instead, the T. reesei Bgl1 has, in domain 1, 3 short anti-parallel β-strands, which together with five parallel β-strands and six α-helices in the same domain, form an incomplete or collapsed α/β barrel.

This structure of domain 3 of T. reesei Bgl1 is similar to that of domain 1 of Thermotoga neapolitana beta-glucosidase 3B. Indeed, when domain 3 of Trichoderma reesei Bgl1 and domain 1 of Thermotoga neapolitana beta-glucosidase 3B are superimposed, a low RMSD value of 1.04 Å was obtained over 113 equivalent Cα positions. What differentiates the domain 3 of T. reesei bgl1 and T. neapolitana beta-glucosidase 3B appears to be in the region where the β-strands lysine 581 to threonine 592 and valine 614 to serine 624 of T. reesei Bgl1 are connected. It appears that the 2 corresponding β-strands in T. neapolitana beta-glucosidase 3B are connected with a short loop whereas in Trichoderma reesei Bgl1, a larger structured insertion, Ala593-Asn613, is present at this position.

The 3-D structure of Trichoderma reesei beta-xylosidase 3A (Xyl3A) been determined at an 1.8 Å resolution using X-ray crystallography. Two ligand datasets were also collected on the improved crystals soaked with xylose and 4-thioxylosbiose, respectively.

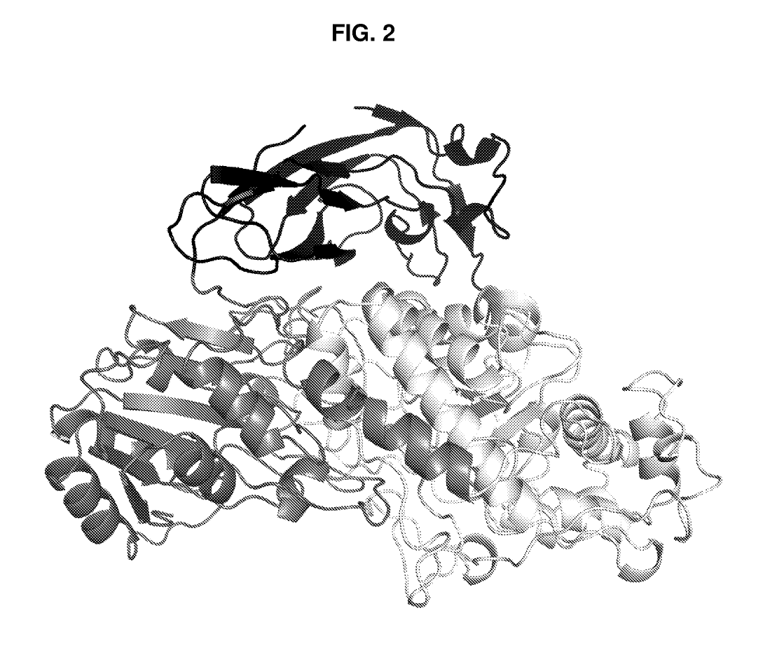

It appears that Xyl3A is a glycosylated three-domain protein of 777 amino acid residues in length. FIG. 2 depicts the Xyl3A structure. Just like the structure of T. reesei Bgl1 as described above, Xyl3A also has three distinct domains with similar domain architecture as reported for Thermotoga neapolitana beta-glucosidase 3B. (see, Pozzo et al., supra). The structure of Xyl3A is also similar to that of Kluyveromyces marxianus beta-glucosidase I, although it is noted that both Xyl3A and Thermotoga neapolitana beta-glucosidase 3B lack the PA14 domain, which is present in domain 2 of Kluyveromyces marxianus beta-glucosidase I. See, Yoshida E., et al., (2010) Role of a PA14 Domain in Determining Substrate Specificity of a Glycosyl Hydrolyase Family 3 Beta-glucosidase from Kluyveromyces marxianus, Biochem. J. 431(1):39-49.

The active site of Xyl3A is located in the interface between domains 1 and 2. Two of the active site residues, the glutamic acid 492 and tyrosine 429 are located in domain 2. The nucleophile aspartic acid 291 is located in domain 1, as are most of the other active site residues including proline 15, leucine 17, glutamic acid 89, tyrosine 152, arginine 166, lysine 206, histidine 207, arginine 221, tyrosine 257, lysine 206 and histidine 207, which together form part of a conserved motif with cis-peptide bonds after lysine 206 (between residues 206 and 207) and after phenylalanine 208 (between residues 208 and 209). See, Harvey A J, Hrmova M, De Gori R, Varghese J N, Fincher G B. 2000 Comparative modeling of the three-dimensional structures of family 3 glycoside hydrolases. Proteins. 2000 Nov. 1; 41 (2):257-69); Pozzo et al., supra. At the individual reside level, however, only lysine 206, histidine 207 and aspartic acid 291 residues are conserved throughout the beta-xylosidases. In addition, glutamic acid 89, which forms hydrogen bonding to the OH-4 group of a xylose residue in subsite-1 appears to be conserved among fungal beta-xylosidases. In most beta-glucosidases the corresponding residue appears to be an aspartic acid.

It appears that the active site of Trichoderma reesei Xyl3A is narrower than that of the Thermotoga neapolitana beta-glucosidase 3B, or that of the Kluyveromyces marxianus beta-glucosidase I. This narrowing appears to be contributed to by residues such as glutamate 14, proline 15, leucine 17 and leucine 22 from the N-terminal region of Xyl3A. The backbone amide of leucine 22 and the backbone carbonyl of leucine 17 appear to form a small water mediated hydrogen bond network with the O1 hydroxyl group of the +1 xylose residue in the 4-thioxylobiose complex with Xyl3A. Tryptophan 87 is located next to leucine 22 and within van der Waal (vdW) distance from both the −1 and +1 subsites. Moreover, the tryptophan 87 has no corresponding residue in any of the GH3 enzymes with known structure. In both the xylose-bound and the 4-thioxylobiose-bound Xyl3A structure models, the sidechains of tryptophan 87 has vdW interactions with the C5 atom of the xylose bound in subsite −1 and fills the space where a C6 atom. It is thought that the O6 hydroxyl group of the glucose can be located in the same space if the xylose was substituted with glucose.

Also the sulfur atom of cysteine 292, which forms a cysteine bridge with cysteine 324, is within vdW distance of the ligand C5 atom in −1. While the sidechain of cysteine 292 points in another direction, the backbone atoms of that cysteine superpose to a large extent with those of tryptophan 286 in Kluyveromyces marxianus beta-glucosidase I, which has been suggested to form one of the edges in a “molecular clamp” around the +1 subsite of the Kluyveromyces marxianus beta-glucosidase I. See, Yoshida E, et al. (2010) Role of a PA14 domain in determining substrate specificity of a glycoside hydrolase family 3 β-glucosidase from Kluyveromyces marxianus. Biochem J. 2010 Oct. 1; 431(1):39-49. Trichoderma reesei Xyl3A therefore does not have such a clamp structure; rather its +1 subsite is surrounded by residues on three sides.

The glutamate 89 of Trichoderma reesei Xyl3A corresponds to the key residue aspartate 58 in Thermotoga neapolitana beta-glucosidase 3B, which has shown to be conserved in about 200 glycosyl hydrolase family 3 enzymes (Pozzo, et al., supra). In the corresponding homologs, this residue was believed to be involved in maintaining correct stereochemistry for the glucose residue bound in subsite-1. The tryptophan 87 residue of Trichoderma reesei Xyl3A may have caused the backbone to move slightly from the familiar corresponding position as generated by aspartate 58 of Thermotoga neapolitana beta-glucosidase 3B, thus making it inappropriate to have an aspartic acid residue at the same position in Xyl3A because its side chains would be too short to help maintain such correct stereochemistry. Therefore, glutamate 89 fills the corresponding position instead, with its side chains forming hydrogen bonds to both the xylose substrate and to the lysine 206 nearby, in order to strengthen the interactions through the interactions among the 3 residues, of this particular site in the enzyme.

### Engineered GH3 Polypeptides Having Both Beta-Glucosidase Activity and Beta-Xylosidase Activity

Three amino acid residues have been identified that contribute to the specificity differences between Trichoderma reesei Bgl1 and Xyl3A. For Trichoderma reesei Bgl1 the corresponding residues are valine 43, tryptophan 237, and methionine 255.

For Trichoderma reesei Bgl1, it is proposed that a change of valine 43 to a larger hydrophobic residue, for example, with a leucine, phenylalanine, or tryptophan, might restrict the binding of glucose at its C6-hydroxyl. Moreover, it is proposed that with the change of valine 43, the tryptophan 237 should be changed to a residue having smaller hydrophobic side chain such as, for example, a leucine, isoleucine, valine, alanine or glycine. Furthermore, the change of valine also may require the introduction of an active site disulfide bridge for example by replacing the methionine at position 255.

Contemplated herein are variants of sequences derived from various organisms wherein the substitution is at residues 43, 237, and 255, which residues are numbered in reference to the amino acid sequence of SEQ ID NO:3. Such organisms include, but are not limited to, Trichoderma reesei, Chaetomium globosum, Aspergillus terreus, Septoria lycopersici, Periconia sp. BCC 2871, Penicillium brasilianus, Phaeosphaeria avenaria, Aspergillus fumigatus, Aspergillus aculeatus, Talaromyces emersonii, Thermoascus aurentiacus, Aspergillus oryzae, Aspergillus niger, Kuraishia capsulata, Uromyces fabae, Saccharomycopsis fibuligera, Coccidioides immitis, Piromyces sp. E2, and Hansenula anomala. For example, as shown in Example 6, analysis indicated that the majority of aligned sequences from Trichoderma reesei, Chaetomium globosum, Aspergillus terreus, Septoria lycopersici, Periconia sp. BCC 2871, Penicillium brasilianus, Phaeosphaeria avenaria, Aspergillus fumigatus, Aspergillus aculeatus, Talaromyces emersonii, Thermoascus aurentiacus, Aspergillus oryzae, Aspergillus niger, Kuraishia capsulata, Uromyces fabae, Saccharomycopsis fibuligera, Coccidioides immitis, Piromyces sp. E2, and Hansenula anomala had a valine at the position corresponding to Bgl1 residue 43. Accordingly, improved properties observed from the study of T. reesei Bgl1 V43L variant herein may be applied to the other GH3 beta-glucosidases having a sequence identity to SEQ ID NO:2 or 3 at a level as low as 31% (Table 9). Accordingly, contemplated herein are variants of polypeptide sequences derived from organisms including, but not limited to, Trichoderma reesei, Chaetomium globosum, Aspergillus terreus, Periconia sp. BCC 2871, Penicillium brasilianus, Phaeosphaeria avenaria, Aspergillus fumigatus, Aspergillus aculeatus, Talaromyces emersonii, Thermoascus aurentiacus, Aspergillus oryzae, Aspergillus niger, Uromyces fabae, Saccharomycopsis fibuligera, Saccharomycopsis fibuligera, Coccidioides immitis, or Piromyces sp. E2. wherein the substitution is at residues 43, 237, and 255, which residues are numbered in reference to the amino acid sequence of SEQ ID NO:3.

### Engineered GH3 Beta-Glucosidase Polypeptides and Polynucleotides Encoding Such Polypeptides

In one aspect, the present compositions and methods provide an engineered GH3 beta-glucosidase polypeptide, fragments thereof, or variants thereof comprising an amino acid sequence that is at least 70% (e.g., at least 70%, at least 75%, at least 80%, at least 85%, at least 90%, at least 91%, at least 92%, at least 93%, at least 94%, at least 95%, at least 96%, at least 97%, at least 98%, at least 99%) identical to SEQ ID NO:2 or SEQ ID NO:3, comprising one or more substitutions at positions 43, 237 and 255, which are numbered in reference to SEQ ID NO:3. In one embodiment, the engineered beta-glucosidase polypeptide retains at least about 95%, at least about 90%, at least about 85%, at least about 80%, at least about 75%, at least about 70%, at least about 65%, at least about 60%, at least about 55%, at least about 50%, at least about 45%, at least about 40%, at least about 30% of the beta-glucosidase activity as compared to the parent, unengineered beta-glucosidase polypeptide. The engineered beta-glucosidase polypeptide also has at least about 5% (e.g., at least about 5%, at least about 10%, at least about 15%, at least about 20%, or higher) beta-xylosidase activity relative to the beta-xylosidase activity of Trichoderma reesei Xyl3A using either one of the standard beta-xylosidase activity assays: the pNpX-hydrolysis assay. In another embodiment, the engineered beta-glucosidase polypeptide not only retains substantially all of the beta-glucosidase activity as compared to the parent, unengineered beta-glucosidase polypeptide, but is a better beta-glucosidase than the parent beta-glucosidase, in that the engineered beta-glucosidase polypeptide has increased beta-glucosidase polypeptide, or improved thermoactivity (i.e., higher activity at higher reaction temperatures), broader pH-activity profile or a pH profile that renders it more suitable as a lignocellulosic biomass hydrolysis enzyme, or has reduced or is less susceptible to product inhibition. The engineered beta-glucosidase polypeptide at the same time acquires at least about 5% (e.g., at least about 5%, at least about 10%, at least about 15%, at least about 20%, or higher) beta-xylosidase activity relative to the beta-xylosidase activity of Trichoderma reesei Xyl3A using either one of the standard beta-xylosidase activity assays: the pNpX-hydrolysis assay.

The beta-glucosidase activity can be measured using two alternative assays. The first is one measuring the hydrolysis of model substrate chloro-nitro-phenyl-beta-D-glucoside (CNPG) or para-nitrophenol-beta-D-glucoside (PNPG). It is called CNPG-hydrolysis assay or PNPG-hydrolysis assay, and both are known to and readily practiced by those skilled in the art. An example of a standard CNPG assay can be found in published patent application WO2011063308. The second is one measuring the cellobiase activity of the beta-glucosidase enzyme, and as such it is called the cellobiase activity assay. Examples of cellobiase activity assays of beta-glucosidases can be found in published patent application WO2011063308.

The beta-xylosidase activity is measured using a standard assay measuring the hydrolysis of model substrate p-nitrophenyl-β-xylopyranoside. The hydrolysis reaction can be followed using ¹H-NMR analysis during the course of the reaction. The experimental methods are described in, e.g., Pauly et al., 1999, Glycobiology 9:93-100.

In some embodiments, the engineered GH3 beta-glucosidase polypeptide, fragments thereof, or variants thereof comprises an amino acid sequence that is at least 35% identical to SEQ ID NO:2 or SEQ ID NO:3, comprising one or more substitutions at positions 43, 237 and 255, which are numbered in reference to SEQ ID NO:3. When the substitution is at position 43, it is the replacement of a valine (V) residue at that position with a tryptophan (W), phenylalanine (F), or leucine (L). When the substitution is at position 237, it is the replacement of a tryptophan (F) residue at that position with a leucine (L), isoleucine (I), valine (V), alanine (A), glycine (G) or cysteine (C). When the substitution is at position 255, it is the replacement of a methionine (M) residue at that position with a cysteine (C). Suitable polypeptide sequences which may comprise one or more substitutions at positions 43, 237 and 255 include polypeptide sequences having at least 35% (e.g., at least 35%, at least 40%, at least 45%, at least 50%, at least 55%, at least 60%, at least 65%, at least 70%, at least 75%, at least 80%, at least 85%, at least 90%, at least 95%, or higher) identity to SEQ ID NO: 37, 38, 39, 41, 43, 44, 45, 46, 47, 48, 49, 50, 51, 53, 54, 55, 56, or 57 wherein the positions are numbered in reference to the mature sequence of Bgl1, SEQ ID NO:3. In embodiments, such polypeptides comprise a substitution of a valine residue at position 43 with a tryptophan (W), phenylalanine (F), or leucine (L), wherein the positions are numbered in reference to the mature sequence of Bgl1, SEQ ID NO:3. Accordingly, provided herein are polypeptides having the amino acid sequence of SEQ ID NO: 37, 38, 39, 41, 43, 44, 45, 46, 47, 48, 49, 50, 51, 53, 54, 55, 56, or 57 and further comprising, for example, a substitution of a valine residue at position 43 with a leucine, wherein the position is numbered in reference to SEQ ID NO: 3.

In some embodiments, the engineered GH3 beta-glucosidase, fragments thereof, or variants thereof comprises an amino acid sequence of at least 35% identity (e.g., at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% identity) to SEQ ID NO:2 or SEQ ID NO:3, with two or more substitutions at the enumerated positions, all numbered in reference to SEQ ID NO:3. For example, the two or more substitutions are at positions 43 and 237. Alternatively the two or more substitutions are at positions 43 and 255. Furthermore, the two or more substitutions can be at positions 237 and 255. In some particular embodiments, the engineered GH3 beta-xylosidase, fragments thereof, or variants thereof comprises an amino acid sequence that is at least 35% identity (e.g., at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% identity) to SEQ ID NO:2 or SEQ ID NO:3, with substitutions at all three positions, namely positions 43, 237 and 255, which are numbered in reference to SEQ ID NO:3. In any of the embodiments described above, the substitution at position 43 may be with a tryptophan (W), phenylalanine (F), or leucine (L). The substitution at position 237 may be with a leucine (L), isoleucine (I), valine (V), alanine (A), glycine (G) or cysteine (C). The substitution at position 255 may be with a cysteine (C).

In some embodiments, the engineered GH3 beta-glucosidase comprises an amino acid sequence that is at least 40% identity (e.g., at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% identity) to SEQ ID NO:2 or SEQ ID NO:3, with the substitutions V43W/F/L, V43W/W237L, V43W/W237I, V43W/W237V, V43W/W237G, V43F/W237L, V43F/W237I, V43F/W237V, V43F/W237A, V43F/W237G, V43L/W237L, V43L/W237I, V43L/W237V, V43L/W237A, V43L/W237G, V43W/W237C/M255C, V43F/W237C/M255C, or V43L/W237C/M255C, wherein the residues are numbered in reference to SEQ ID NO:3

In certain embodiments, the engineered GH3 beta-glucosidase comprising an amino acid sequence that is at least 35% identity to SEQ ID NO: 2 or SEQ ID NO:3 and one or more substitutions at positions 43, 237 and 255, has detectable beta-xylosidase activity. In some embodiments, the engineered beta-glucosidase has at least 2% (e.g., at least 5%, at least 10%, at least 15%, or at least 20% or higher) of the beta-xylosidase activity of purified Trichoderma reesei beta-xylosidase 3 (Xyl3A) as measured using a standard assay measuring the hydrolysis of model substrate para-nitrophenol-beta-D-xyloside (pNpX). In some embodiments, the engineered beta-glycosides has at least 2% higher (e.g., 2% higher, 5% higher, 10% higher, 15% higher, or even 20% higher) beta-xylosidase activity than that of its native, unengineered, parent beta-glucosidase. In some embodiments, the engineered beta-glucosidase retains substantial level of beta-glucosidase activity, for example, at least 95%, at least 90%, at least 85%, at least 80%, at least 75%, at least 70%, at least 65%, at least 60%, at least 55%, at least 50%, at least 45%, at least 40%, at least 35%, or at least 30%, of its parent unengineered beta-glucosidase, while acquiring increased beta-xylosidase activity. In other embodiments, the engineered beta-glucosidase not only retains substantial level of beta-glucosidase activity of its parent unengineered beta-glucosidase, but is a better or improved beta-glucosidase as compared to its parent unengineered beta-glucosidase in that it has increased beta-glucosidase and/or cellobiase activity, or has higher thermoactivity (i.e., higher enzymatic activity at a higher temperature), or has a broader or more useful pH-activity profile for lignocellulosic biomass hydrolysis, or has a reduced or is less susceptible to product inhibition.

In some embodiments, the engineered GH3 beta-glucosidase polypeptide is a variant GH3 polypeptide having a specific degree of amino acid sequence identity to the exemplified Trichoderma reesei beta-glucosidase 1 (Bgl1) polypeptide, e.g., at least 35% (e.g., at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99%) sequence identity to the amino acid sequence of SEQ ID NO:2 or to the mature sequence SEQ ID NO:3, and comprising one or more substitutions at the positions 43, 237, and 255, wherein the numbering of the positions are in reference to SEQ ID NO:3. Sequence identity can be determined by amino acid sequence alignment, e.g., using a program such as BLAST, ALIGN, or CLUSTAL, as described herein.

In certain embodiments, the engineered GH3 beta-glucosidase polypeptides, which have both the beta-glucosidase activity and the beta-xylosidase activity, are produced recombinantly, in a microorganism, for example, in a bacterial or fungal host organism, while in others the engineered GH3 beta-glucosidase polypeptides, which have both the beta-glucosidase activity and the beta-xylosidase activity, can be produced synthetically.

In certain embodiments, the engineered GH3 beta-glucosidase polypeptide which has both beta-glucosidase and beta-xylosidase activity, aside from the substitutions at one or more of positions 43, 237, and 255, which numbering is in reference to SEQ ID NO:3, may also include substitutions that do not substantially affect the structure, function, and/or specificity of the polypeptide. Examples of these substitutions are conservative mutations, as summarized in Table I.

TABLE I
Amino Acid Substitutions

| Original | | |
|---|---|---|
| Residue | Code | Acceptable Substitutions |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, beta-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4- carboxylic acid, D-or |
| | | L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Substitutions can be made by mutating a nucleic acid encoding a select GH3 parent beta-glucosidase enzyme, and then expressing the variant polypeptide in an organism. Certain non-naturally occurring amino acids or chemical modifications of amino acids can also be included, but those are typically made by chemically modifying an engineered GH3 beta-glucosidase polypeptide with the desired substitutions that has been synthesized by an organism.

Other modifications, including other substitutions, insertions or deletions that do not significantly affect the structure, function, expression or specificity of the polypeptide, to an engineered GH3 parent beta-glucosidase in accordance with the embodiments above, comprising a sequence that is at least 35% identical to SEQ ID NO:2 or SEQ ID NO:3, and one or more substitutions at 43, 237, and 255, can also be applied with the methods and compositions herein.

Engineered GH3 beta-glucosidase may be fragments of “full-length” engineered GH3 beta-glucosidase that retain the beta-glucosidase activity, or have increased or improved beta-glucosidase and/or cellobiase activity, and the newly acquired beta-xylosidase activity. Preferably those functional fragments (i.e., fragments that retain at least some beta-glucosidase activity and at least some of the acquired beta-xylosidase activity) are at least 80 amino acid residues in length (e.g., at least 80 amino acid residues, at least 100 amino acid residues, at least 120 amino acid residues, at least 140 amino acid residues, at least 160 amino acid residues, at least 180 amino acid residues, at least 200 amino acid residues, at least 220 amino acid residues, at least 240 amino acid residues, at least 260 amino acid residues, at least 280 amino acid residues, at least 300 amino acid residues in length or longer). Such fragments suitably retain the active site of the full-length precursor polypeptides or full length mature polypeptides but may have deletions of non-critical amino acid residues. The activity of fragments can be readily determined using the methods of measuring beta-glucosidase activity and beta-xylosidase activity as described herein, or by other suitable assays or other means of activity measurements known in the art.

In some embodiments, the engineered GH3 beta-glucosidase amino acid sequences and derivatives are produced as an N- and/or C-terminal fusion protein, for example, to aid in extraction, detection and/or purification and/or to add functional properties to the engineered GH3 beta-glucosidase polypeptides. Examples of fusion protein partners include, but are not limited to, glutathione-S-transferase (GST), 6×His, GAL4 (DNA binding and/or transcriptional activation domains), FLAG-, MYC-tags or other tags known to those skilled in the art. In some embodiments, a proteolytic cleavage site is provided between the fusion protein partner and the polypeptide sequence of interest to allow removal of fusion sequences. Suitably, the fusion protein does not hinder the beta-glucosidase activity and the acquired beta-xylosidase activity of the engineered GH3 beta-glucosidase polypeptide. In some embodiments, the engineered GH3 beta-glucosidase polypeptide is fused to a functional domain including a leader peptide, propeptide, binding domain and/or catalytic domain. Fusion proteins are optionally linked to the engineered GH3 beta-glucosidase polypeptide through a linker sequence that joins the engineered GH3 beta-glucosidase polypeptide and the fusion domain without significantly affecting the properties of either component. The linker optionally contributes functionally to the intended application.

In a related aspect, the engineered GH3 beta-glucosidase having also beta-xylosidase activity, is encoded by a polynucleotide having at least about 35% identity (e.g., at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% identity) to SEQ ID NO:1, whereby the polynucleotide also encodes certain substitution amino acid residues at positions 43, 237 and 255, with reference to SEQ ID NO:3. The polynucleotide encodes an engineered GH3 beta-glucosidase that has at least 2% (e.g., at least 5%, at least 10%, at least 15%, or at least 20% or higher) of the beta-xylosidase activity of purified Trichoderma reesei beta-xylosidase 3 (Xyl3A) as measured using a standard assay measuring the hydrolysis of model substrate para-nitrophenol-beta-D-xyloside (pNpX). In some embodiments, the polynucleotide encodes an engineered GH3 beta-glucosidase that has at least 2% higher (e.g., at least 2% higher, at least 5% higher, at least 10% higher, at least 15% higher, at least 20%) beta-xylosidase activity than that of its parent, unengineered, beta-glucosidase. The engineered GH3 beta-glucosidase may also retain substantial level of beta-glucosidase activity, for example, at least 95%, at least 90%, at least 85%, at least 80%, at least 75%, at least 70%, at least 65%, at least 60%, at least 55%, at least 50%, at least 45%, at least 40%, at least 35%, or at least 30%, of its parent unengineered beta-glucosidase, while acquiring increased beta-xylosidase activity. In an alternative embodiment, the engineered GH3 beta-glucosidase may not only retain substantial level of beta-glucosidase activity of its parent unengineered beta-glucosidase, but also be a better or improved beta-glucosidase in that it has increased levels of beta-glucosidase and/or cellobiase activity, or it has increased thermoactivity (i.e., higher enzymatic activity at higher temperature), or it has broader or more suitable pH activity optimum for lignocellulosic biomass hydrolysis, or it has reduced or is less susceptible to product inhibition.

In some embodiments, the engineered GH3 beta-glucosidase is encoded by a polynucleotide having at least 35% identity (e.g., at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% identity) to SEQ ID NO:1, whereby the polynucleotide also encodes one of the following substitutions: V43W/F/L, V43W/W237L, V43W/W237I, V43W/W237V, V43W/W237G, V43F/W237L, V43F/W237I, V43F/W237V, V43F/W237A, V43F/W237G, V43L/W237L, V43L/W237I, V43L/W237V, V43L/W237A, V43L/W237G, V43W/W237C/M255C, V43F/W237C/M255C, or V43L/W237C/M255C, the numbering of the residues being in reference to SEQ ID NO:3. The engineered GH3 beta-glucosidase has at least 2% (e.g., at least 5%, at least 10%, at least 15%, or at least 20% or higher) of the beta-xylosidase activity of purified Trichoderma reesei beta-xylosidase 3 (Xyl3A) as measured using a standard assay measuring the hydrolysis of model substrate para-nitrophenol-beta-D-xyloside (pNpX). In some embodiments, the engineered GH3 beta-glucosidase has at least 2% higher (e.g., at least 2%, at least 5%, at least 10%, at least 15%, at least 20% higher) beta-xylosidase activity as compared to that of the native, unengineered, parent beta-glucosidase. Moreover, the engineered GH3 beta-glucosidase retains substantial level of beta-xylosidase activity, for example, at least 95%, at least 90%, at least 85%, at least 80%, at least 75%, at least 70%, at least 65%, at least 60%, at least 55%, at least 50%, at least 45%, at least 40%, at least 35%, or at least 30%, of its parent unengineered beta-glucosidase, while acquiring increased beta-xylosidase activity. In an alternative embodiment, the engineered GH3 beta-glucosidase may not only retain substantial level of beta-glucosidase activity of its parent unengineered beta-glucosidase, but also be a better or improved beta-glucosidase in that it has increased levels of beta-glucosidase and/or cellobiase activity, or it has increased thermoactivity (i.e., higher enzymatic activity at higher temperature), or it has broader or more suitable pH activity optimum for lignocellulosic biomass hydrolysis, or it has reduced or is less susceptible to product inhibition.

In certain embodiments, the engineered GH3 beta-glucosidase is encoded by a polynucleotide having at least 35% (e.g., at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% identity) identity to SEQ ID NO:1, or hybridizes under medium stringency conditions, high stringency conditions, or very high stringency conditions to SEQ ID NO:1, or to a complementary sequence thereof, whereby the polynucleotide also encodes certain amino acid substitutions at residues 43, 237 and 255 of SEQ ID NO:3. In some embodiments, the amino acid substitution is selected from one of the following: V43W/F/L, V43W/W237L, V43W/W237I, V43W/W237V, V43W/W237G, V43F/W237L, V43F/W237I, V43F/W237V, V43F/W237A, V43F/W237G, V43L/W237L, V43L/W237I, V43L/W237V, V43L/W237A, V43L/W237G, V43W/W237C/M255C, V43F/W237C/M255C, or V43L/W237C/M255C. In some embodiments, the engineered GH3 beta-glucosidase has at least 2% (e.g., at least 5%, at least 10%, at least 15%, or at least 20% or higher) of the beta-xylosidase activity of purified Trichoderma reesei beta-xylosidase 3 (Xyl3A) as measured using a standard assay measuring the hydrolysis of model substrate para-nitrophenol-beta-D-xyloside (pNpX). In some embodiments, the engineered GH3 beta-glucosidase has at least 2% higher (e.g., at least 2% higher, at least 5% higher, at least 10% higher, at least 15% higher, or even at least 20% higher) beta-xylosidase activity than that of its native, unengineered, parent beta-glucosidase. Moreover, the engineered GH3 beta-glucosidase retains substantial level of beta-glucosidase activity, for example, at least 95%, at least 90%, at least 85%, at least 80%, at least 75%, at least 70%, at least 65%, at least 60%, at least 55%, at least 50%, at least 45%, at least 40%, at least 35%, or at least 30%, of its parent unengineered beta-glucosidase, while acquiring increased beta-xylosidase activity. In an alternative embodiment, the engineered GH3 beta-glucosidase may not only retain substantial level of beta-glucosidase activity of its parent unengineered beta-glucosidase, but also be a better or improved beta-glucosidase in that it has increased levels of beta-glucosidase and/or cellobiase activity, or it has increased thermoactivity (i.e., higher enzymatic activity at higher temperature), or it has broader or more suitable pH activity optimum for lignocellulosic biomass hydrolysis, or has reduced or is less susceptible to product inhibition.

In some embodiments, the polynucleotide that encodes an engineered GH3 beta-glucosidase polypeptide is fused in frame behind (i.e., downstream of) a coding sequence for a signal peptide for directing the extracellular secretion of the engineered GH3 beta-glucosidase polypeptide. As described herein, the term “heterologous” when used to refer to a signal sequence used to express a polypeptide of interest, it is meant that the signal sequence and the polypeptide of interest are from different organisms. Heterologous signal sequences include, for example, those from other fungal cellulase genes, such as, e.g., the signal sequence of Trichoderma reesei CBH1. Expression vectors may be provided in a heterologous host cell suitable for expressing an engineered GH3 beta-xylosidase polypeptide, or suitable for propagating the expression vector prior to introducing it into a suitable host cell.

In some embodiments, polynucleotides encoding the engineered GH3 beta-glucosidase polypeptides hybridize to the polynucleotide of SEQ ID NO:1 (or to the complement thereof) under specified hybridization conditions. Examples of conditions are intermediate stringency, high stringency and extremely high stringency conditions, which are described herein.

The engineered beta-glucosidase polynucleotides may be synthetic (i.e., man-made), and may be codon-optimized for expression in a different host, mutated to introduce cloning sites, or otherwise altered to add functionality.

The nucleic acid sequence encoding the coding region of a representative engineered beta-glucosidase Trichoderma reesei Bgl1 polypeptide is below (SEQ ID NO:1):

ATGCGCTACCGCACCGCTGCCGCTTTAGCCTTAGCCACCGGCCCCTTCG
CCAGAGCCGATAGCCACAGCACCTCCGGCGCTAGTGCTGAAGCTGTTGT
CCCTCCTGCTGGCACCCCTTGGGGCACCGCCTACGACAAGGCCAAGGCC
GCCCTCGCCAAGCTCAACCTCCAGGACAAGGTCGGCATCGTCAGCGGCG
TCGGCTGGAACGGCGGTCCCTGCGTCGGCAACACCAGCCCCGCCAGCAA
GATCAGCTACCCCAGCCTCTGCCTCCAGGACGGCCCCCTCGGCGTCCGC
TACAGCACCGGCAGCACCGCCTTCACCCCTGGCGTCCAGGCCGCCAGCA
CCTGGGACGTCAACCTCATCCGCGAGCGCGGCCAGTTCATCGGCGAAGA
GGTCAAGGCCAGCGGCATCCACGTCATCCTCGGTCCCGTTGCTGGTCCC
TTAGGCAAGACCCCCCAGGGCGGTCGCAACTGGGAGGGCTTCGGCGTCG
ACCCCTACCTCACCGGCATTGCCATGGGCCAGACCATCAACGGCATCCA
GAGCGTCGGCGTCCAGGCCACCGCCAAGCACTACATCCTCAACGAGCAA
GAGTTAAACCGCGAGACTATCAGCAGCAACCCCGACGACCGCACCCTCC
ACGAGTTATACACCTGGCCCTTCGCCGACGCCGTCCAGGCCAACGTCGC
CAGCGTCATGTGCAGCTACAACAAGGTCAACACCACCTGGGCCTGCGAG
GACCAGTACACCCTCCAGACCGTCCTCAAGGACCAGCTCGGCTTCCCCG
GCTACGTCATGACCGACTGGAACGCCCAGCACACCACCGTCCAGAGCGC
CAACAGCGGCCTCGACATGAGCATGCCCGGCACCGACTTCAACGGCAAC
AACCGCCTCTGGGGCCCTGCCCTCACCAACGCCGTCAACAGCAACCAGG
TCCCCACCTCCCGCGTCGACGACATGGTCACCCGCATCCTCGCCGCCTG
GTACTTAACCGGCCAAGACCAGGCTGGCTATCCCAGCTTCAACATCAGC
CGCAACGTCCAGGGCAACCACAAGACCAACGTCCGCGCCATTGCCCGCG
ACGGCATCGTCCTCCTCAAGAACGACGCCAACATCCTCCCCCTCAAGAA
GCCCGCCTCTATCGCCGTCGTCGGCAGCGCCGCCATCATCGGCAACCAC
GCCCGCAACAGCCCCAGCTGCAACGACAAGGGCTGCGATGACGGTGCCC
TCGGCATGGGCTGGGGCTCTGGCGCCGTCAACTACCCCTACTTCGTCGC
CCCCTACGACGCCATCAACACCCGCGCCAGCAGCCAGGGCACCCAGGTC
ACCCTCAGCAACACCGACAATACTTCTTCTGGCGCTTCTGCTGCTAGAG
GCAAGGACGTCGCCATCGTTTTTATCACTGCCGATTCTGGCGAAGGCTA
CATCACCGTCGAGGGCAACGCCGGCGACCGCAACAACCTCGACCCCTGG
CACAACGGCAATGCCCTCGTCCAGGCCGTTGCTGGTGCTAACAGCAACG
TCATCGTCGTCGTCCACAGCGTCGGCGCCATCATCCTCGAGCAGATCCT
CGCCCTCCCCCAGGTCAAGGCCGTCGTCTGGGCCGGCTTACCCAGCCAG
GAAAGCGGCAACGCCTTAGTCGACGTCCTCTGGGGTGACGTTTCCCCCT
CTGGCAAGCTCGTCTACACCATTGCCAAGAGCCCCAACGACTACAACAC
CCGCATTGTCAGCGGCGGCAGCGACAGCTTCAGCGAGGGCCTCTTCATC
GACTACAAGCACTTCGACGACGCCAACATTACCCCCCGCTACGAGTTCG
GCTACGGCCTCAGCTACACCAAGTTCAACTACAGCCGCCTCAGCGTCCT
CAGCACCGCCAAGAGCGGCCCTGCCACTGGTGCTGTCGTCCCTGGTGGC
CCTTCTGACCTCTTCCAGAACGTCGCCACGGTCACCGTCGACATTGCCA
ACTCCGGCCAGGTCACTGGCGCCGAGGTCGCCCAGCTCTACATCACCTA
CCCCAGCAGCGCCCCTCGCACTCCTCCCAAGCAGCTCAGAGGCTTCGCT
AAGTTAAACTTAACCCCTGGCCAGAGCGGCACCGCCACCTTTAACATCC
GCAGACGCGACCTCAGCTACTGGGACACCGCCAGCCAGAAGTGGGTCGT
CCCCAGCGGCAGCTTCGGCATCTCCGTCGGCGCCAGCTCCCGCGACATC
CGCCTCACCAGCACCCTCAGCGTCGCCTGATGA

As is well known to those of ordinary skill in the art, due to the degeneracy of the genetic code, polynucleotides having significantly different sequences can nonetheless encode identical, or nearly identical, polypeptides. As such, aspects of the present compositions and methods include polynucleotides encoding an engineered GH3 beta-glucosidase polypeptides or derivatives thereof that contain a nucleic acid sequence that is at least 35% identical to SEQ ID NO:1, including at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or even at least 99% identical to SEQ ID NO:1. In some embodiments, the engineered GH3 beta-glucosidase polypeptides contain a nucleic acid sequence that is nearly identical to SEQ ID NO:1.

In some embodiments, polynucleotides may include a sequence encoding a signal peptide. Many convenient signal sequences may be suitably employed.

The present disclosure provides host cells that are engineered to express one or more engineered GH3 beta-glucosidase polypeptides of the disclosure. Suitable host cells include cells of any microorganism (e.g., cells of a bacterium, a protist, an alga, a fungus (e.g., a yeast or filamentous fungus), or other microbe), and are preferably cells of a bacterium, a yeast, or a filamentous fungus.

Suitable host cells of the bacterial genera include, but are not limited to, cells of Escherichia, Bacillus, Lactobacillus, Pseudomonas, and Streptomyces. Suitable cells of bacterial species include, but are not limited to, cells of Escherichia coli, Bacillus subtilis, Bacillus hemicellulosilyticus, Lactobacillus brevis, Pseudomonas aeruginosa, and Streptomyces lividans.

Suitable host cells of the genera of yeast include, but are not limited to, cells of Saccharomyces, Schizosaccharomyces, Candida, Hansenula, Pichia, Kluyveromyces, and Phaffia. Suitable cells of yeast species include, but are not limited to, cells of Saccharomyces cerevisiae, Schizosaccharomyces pombe, Candida albicans, Hansenula polymorpha, Pichia pastoris, P. canadensis, Kluyveromyces marxianus, and Phaffia rhodozyma.

Suitable host cells of filamentous fungi include all filamentous forms of the subdivision Eumycotina. Suitable cells of filamentous fungal genera include, but are not limited to, cells of Acremonium, Aspergillus, Aureobasidium, Bjerkandera, Ceriporiopsis, Chrysoporium, Coprinus, Coriolus, Corynascus, Chaertomium, Cryptococcus, Filobasidium, Fusarium, Gibberella, Humicola, Magnaporthe, Mucor, Myceliophthora, Mucor, Neocallimastix, Neurospora, Paecilomyces, Penicillium, Phanerochaete, Phlebia, Piromyces, Pleurotus, Scytaldium, Schizophyllum, Sporotrichum, Talaromyces, Thermoascus, Thielavia, Tolypocladium, Trametes, and Trichoderma.

Suitable cells of filamentous fungal species include, but are not limited to, cells of Aspergillus awamori, Aspergillus fumigatus, Aspergillus foetidus, Aspergillus japonicus, Aspergillus nidulans, Aspergillus niger, Aspergillus oryzae, Chrysosporium lucknowense, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Neurospora intermedia, Penicillium purpurogenum, Penicillium canescens, Penicillium solitum, Penicillium funiculosum Phanerochaete chrysosporium, Phlebia radiate, Pleurotus eryngii, Talaromyces flavus, Thielavia terrestris, Trametes villosa, Trametes versicolor, Trichoderma harzianum, Trichoderma koningii, Trichoderma longibrachiatum, Trichoderma reesei, and Trichoderma viride.

Methods of transforming nucleic acids into these organisms are known in the art. For example, a suitable procedure for transforming Aspergillus host cells is described in EP 238 023.

In some embodiments, the engineered GH3 beta-glucosidase polypeptide is fused to a signal peptide to, for example, facilitate extracellular secretion of the engineered GH3 beta-glucosidase polypeptide. In particular embodiments, the engineered GH3 beta-glucosidase is expressed in a heterologous organism as a secreted polypeptide. The compositions and methods herein thus encompass methods for expressing an engineered beta-glucosidase polypeptide as a secreted polypeptide in a heterologous organism.

In a specific embodiment, a GH3 beta-glucosidase polypeptide of the invention or an engineered variant thereof, having acquired beta-xylosidase activity, for example, may be a part of an enzyme composition, contributing to the enzymatic hydrolysis process and to the liberation of D-glucose from oligosaccharides such as cellobiose. In certain embodiments, the GH3 beta-glucosidase polypeptide/variant may be genetically engineered to express in an ethanologen, such that the ethanologen microbe expresses and/or secrets such a GH3 beta-glucosidase/beta-xylosidase activity. Moreover, the GH3 polypeptide may be a part of the hydrolysis enzyme composition while at the same time also expressed and/or secreted by the ethanologen, whereby the soluble fermentable sugars produced by the hydrolysis of the lignocellulosic biomass substrate using the hydrolysis enzyme composition is metabolized and/or converted into ethanol by an ethanologen microbe that also expresses and/or secrets the GH3 polypeptide. The hydrolysis enzyme composition can comprise the GH3 beta-glucosidase polypeptide/variant thereof in addition to one or more other cellulases and/or one or more hemicellulases. The ethanologen can be engineered such that it expresses the GH3 beta-glucosidase/variant polypeptide, one or more other cellulases, one or more other hemicellulases, or a combination of these enzymes. One or more of the GH3 beta-glucosidase/variant may be in the hydrolysis enzyme composition and expressed and/or secreted by the ethanologen. For example, the hydrolysis of the lignocellulosic biomass substrate may be achieved using an enzyme composition comprising a GH3 polypeptide or variant of the present invention, and the sugars produced from the hydrolysis can then be fermented with a microorganism engineered to express and/or secret GH3 polypeptide or variant polypeptide, which may or may not be the same polypeptide as the one in the enzyme composition. Alternatively, an enzyme composition comprising a first GH3 beta-glucosidase polypeptide participates in the hydrolysis step and a second GH3 beta-glucosidase, which also has beta-xylosidase activity, which is different from the first beta-glucosidase, is expressed and/or secreted by the ethanologen.

The disclosure also provides expression cassettes and/or vectors comprising the above-described nucleic acids. Suitably, the nucleic acid encoding an engineered GH3 beta-glucosidase polypeptide having both beta-glucosidase activity and beta-xylosidase activity is operably linked to a promoter. Promoters are well known in the art. Any promoter that functions in the host cell can be used for expression of the engineered GH3 beta-glucosidase/variant herein and/or any of the other nucleic acids of the present disclosure. Virtually any promoter capable of driving these nucleic acids can be used.

Specifically, where recombinant expression in a filamentous fungal host is desired, the promoter can be a filamentous fungal promoter. The nucleic acids can be, for example, under the control of heterologous promoters. The nucleic acids can also be expressed under the control of constitutive or inducible promoters. Examples of promoters that can be used include, but are not limited to, a cellulase promoter, a xylanase promoter, the 1818 promoter (previously identified as a highly expressed protein by EST mapping Trichoderma). For example, the promoter can suitably be a cellobiohydrolase, endoglucanase, or beta-glucosidase promoter. A particularly suitable promoter can be, for example, a T. reesei cellobiohydrolase, endoglucanase, or beta-glucosidase promoter. For example, the promoter is a cellobiohydrolase I (cbh1) promoter. Non-limiting examples of promoters include a cbh1, cbh2, egl1, egl2, egl3, egl4, egl5, pki1, gpd1, xyn1, or xyn2 promoter. Additional non-limiting examples of promoters include a T. reesei cbh1, cbh2, egl1, egl2, egl3, egl4, egl5, pki1, gpd1, xyn1, or xyn2 promoter.

The nucleic acid sequence encoding an engineered GH3 beta-glucosidase polypeptide herein can be included in a vector. In some aspects, the vector contains the nucleic acid sequence encoding the engineered GH3 beta-glucosidase polypeptide under the control of an expression control sequence. In some aspects, the expression control sequence is a native expression control sequence. In some aspects, the expression control sequence is a non-native expression control sequence. In some aspects, the vector contains a selective marker or selectable marker. In some aspects, the nucleic acid sequence encoding the engineered GH3 beta-glucosidase polypeptide is integrated into a chromosome of a host cell without a selectable marker.

Suitable vectors are those which are compatible with the host cell employed. Suitable vectors can be derived, for example, from a bacterium, a virus (such as bacteriophage T7 or a M-13 derived phage), a cosmid, a yeast, or a plant. Suitable vectors can be maintained in low, medium, or high copy number in the host cell. Protocols for obtaining and using such vectors are known to those in the art (see, for example, Sambrook et al., Molecular Cloning: A Laboratory Manual, 2^{nd} ed., Cold Spring Harbor, 1989).

In some aspects, the expression vector also includes a termination sequence. Termination control regions may also be derived from various genes native to the host cell. In some aspects, the termination sequence and the promoter sequence are derived from the same source.

A nucleic acid sequence encoding an engineered GH3 beta-glucosidase polypeptide can be incorporated into a vector, such as an expression vector, using standard techniques (Sambrook et al., Molecular Cloning: A Laboratory Manual, Cold Spring Harbor, 1982).

In some aspects, it may be desirable to over-express an engineered GH3 beta-glucosidase polypeptide herein and/or one or more of any other nucleic acid described in the present disclosure at levels far higher than currently found in naturally-occurring cells. In some embodiments, it may be desirable to under-express (e.g., mutate, inactivate, or delete) an endogenous beta-glucosidase and/or one or more of any other nucleic acid described in the present disclosure at levels far below that those currently found in naturally-occurring cells.

### Chemical Synthesis

Alternatively, the engineered GH3 beta-glucosidase, or portions thereof, may be produced by direct peptide synthesis using solid-phase techniques (see, e.g., Stewart et al., Solid-Phase Peptide Synthesis, W.H. Freeman Co., San Francisco, Calif. (1969); Merrifield, J. Am. Chem. Soc., 85:2149-2154 (1963)). In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of an engineered GH3 beta-glucosidase polypeptide may be chemically synthesized separately and combined using chemical or enzymatic methods to produce a full-length GH3 polypeptide.

### Recombinant Methods of Making

DNA encoding an engineered GH3 beta-glucosidase polypeptide as described above may be obtained from oligonucleotide synthesis.

Host cells are transfected or transformed with expression or cloning vectors described herein for the production of engineered GH3 beta-glucosidase polypeptides. The host cells are cultured in conventional nutrient media modified as appropriate for inducing promoters, selecting transformants, or amplifying the genes encoding the desired sequences. The culture conditions, such as media, temperature, pH and the like, can be selected by the ordinarily skilled artisan without undue experimentation. In general, principles, protocols, and practical techniques for maximizing the productivity of cell cultures can be found in Mammalian Cell Biotechnology: a Practical Approach, M. Butler, ed. (IRL Press, 1991) and Sambrook et al., Molecular Cloning: A Laboratory Manual (New York: Cold Spring Harbor Laboratory Press, 1989).

Methods of transfection are known to the ordinarily skilled artisan, for example, CaPO₄ and electroporation. Depending on the host cell used, transformation is performed using standard techniques appropriate to such cells. The calcium treatment employing calcium chloride, as described in Sambrook et al., Molecular Cloning: A Laboratory Manual (New York: Cold Spring Harbor Laboratory Press, 1989), or electroporation is generally used for prokaryotes or other cells that contain substantial cell-wall barriers. Infection with Agrobacterium tumefaciens is used for transformation of certain plant cells, as described by Shaw et al., Gene, 23:315 (1983) and WO 89/05859 published 29 Jun. 1989. Transformations into yeast can be carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, microporation, biolistic bombardment, bacterial protoplast fusion with intact cells, or polycations, e.g., polybrene, polyornithine, may also be used.

Suitable host cells for cloning or expressing the DNA in the vectors herein include prokaryote, yeast, or filamentous fungal cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). In addition to prokaryotes, eukaryotic microorganisms such as filamentous fungi or yeast are suitable cloning or expression hosts for vectors encoding the engineered GH3 beta-glucosidase as described herein. Saccharomyces cerevisiae is a commonly used lower eukaryotic host microorganism.

In some embodiments, the microorganism to be transformed includes a strain derived from Trichoderma sp. or Aspergillus sp. Exemplary strains include T. reesei which is useful for obtaining overexpressed protein or Aspergillus niger var. awamori. For example, Trichoderma strain RL-P37, described by Sheir-Neiss et al. in Appl. Microbiol. Biotechnology, 20 (1984) pp. 46-53 is known to secrete elevated amounts of cellulase enzymes. Functional equivalents of RL-P37 include Trichoderma reesei (longibrachiatum) strain RUT-C30 (ATCC No. 56765) and strain QM9414 (ATCC No. 26921). Another example includes overproducing mutants as described in Ward et al. in Appl. Microbiol. Biotechnology 39:738-743 (1993). For example, it is contemplated that these strains would also be useful in overexpressing an engineered GH3 beta-glucosidase polypeptide, or a variant thereof. The selection of the appropriate host cell is deemed to be within the skill in the art.

### Preparation and Use of a Replicable Vector

DNA encoding an engineered GH3 beta-glucosidase polypeptide or derivatives thereof (as described above) can be prepared for insertion into an appropriate microorganism. According to the present compositions and methods, DNA encoding the engineered GH3 beta-glucosidase polypeptide includes all of the DNA necessary to encode for a protein which has functional engineered GH3 beta-glucosidase having at least some retained beta-glucosidase activity of the parent but also acquired at least some beta-glucosidase activity. As such, embodiments of the present compositions and methods include DNA encoding an engineered GH3 beta-glucosidase polypeptide that has both beta-glucosidase activity and beta-xylosidase activity.

The DNA encoding the engineered GH3 beta-glucosidase may be prepared by the construction of an expression vector carrying the DNA encoding such an engineered enzyme. The expression vector carrying the inserted DNA fragment encoding the GH3 polypeptide may be any vector which is capable of replicating autonomously in a given host organism or of integrating into the DNA of the host, typically a plasmid, cosmid, viral particle, or phage. Various vectors are publicly available. It is also contemplated that more than one copy of DNA encoding an engineered GH3 beta-glucosidase may be recombined into the strain to facilitate overexpression.

In certain embodiments, DNA sequences for expressing the engineered GH3 beta-glucosidase polypeptide as described herein above include the promoter, gene coding region, and terminator sequence all originate from the native gene to be expressed. Gene truncation may be obtained by deleting away undesired DNA sequences (e.g., coding for unwanted domains) to leave the domain to be expressed under control of its native transcriptional and translational regulatory sequences. A selectable marker can also be present on the vector allowing the selection for integration into the host of multiple copies of the GH3 beta-glucosidase gene sequence.

In other embodiments, the expression vector is preassembled and contains sequences required for high level transcription and, in some cases, a selectable marker. It is contemplated that the coding region for a gene or part thereof can be inserted into this general purpose expression vector such that it is under the transcriptional control of the expression cassette's promoter and terminator sequences. For example, pTEX is such a general purpose expression vector. Genes or part thereof can be inserted downstream of the strong cbh1 promoter.

In the vector, the DNA sequence encoding the engineered GH3 polypeptides of the present compositions and methods should be operably linked to transcriptional and translational sequences, e.g., a suitable promoter sequence and signal sequence in reading frame to the structural gene. The promoter may be any DNA sequence which shows transcriptional activity in the host cell and may be derived from genes encoding proteins either homologous or heterologous to the host cell. The signal peptide provides for extracellular production (secretion) of the engineered GH3 polypeptide or derivatives thereof. The DNA encoding the signal sequence can be that which is naturally associated with the gene to be expressed. However the signal sequence from any suitable source, for example an exo-cellobiohydrolases or endoglucanase from Trichoderma, a xylanase from a bacterial species, e.g., from Streptomyces coelicolor, etc., are contemplated in the present compositions and methods.

The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

A desired engineered GH3 beta-glucosidase as provided herein may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector or it may be a part of the GH3 polypeptide-encoding DNA that is inserted into the vector. The signal sequence may be a prokaryotic signal sequence selected, for example, from the group of the alkaline phosphatase, penicillinase, lpp, or heat-stable enterotoxin II leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990.

Both expression and cloning vectors may contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria and the 2μ plasmid origin is suitable for yeast.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli. A suitable selection gene for use in yeast is the trp1 gene present in the yeast plasmid YRp7 (Stinchcomb et al., Nature, 282:39 (1979); Kingsman et al., Gene, 7:141 (1979); Tschemper et al., Gene, 10:157 (1980)). The trp1 gene provides a selection marker for a mutant strain of yeast lacking the ability to grow in tryptophan, for example, ATCC No. 44076 or PEP4-1 (Jones, Genetics, 85:12 (1977)). An exemplary selection gene for use in Trichoderma sp is the pyr4 gene.

Expression and cloning vectors usually contain a promoter operably linked to the engineered GH3 polypeptide-encoding nucleic acid sequence. The promoter directs mRNA synthesis. Promoters recognized by a variety of potential host cells are well known. Promoters include a fungal promoter sequence, for example, the promoter of the cbh1 or egl1 gene.

Promoters suitable for use with prokaryotic hosts include the β-lactamase and lactose promoter systems (Chang et al., Nature, 275:615 (1978); Goeddel et al., Nature, 281:544 (1979)), alkaline phosphatase, a tryptophan (trp) promoter system (Goeddel, Nucleic Acids Res., 8:4057 (1980); EP 36,776), and hybrid promoters such as the tac promoter (deBoer et al., Proc. Natl. Acad. Sci. USA, 80:21-25 (1983)). Additional promoters, e.g., the A4 promoter from A. niger, also find use in bacterial expression systems, e.g., in S. lividans. Promoters for use in bacterial systems also may contain a Shine-Dalgarno (S.D.) sequence operably linked to the DNA encoding an engineered GH3 beta-glucosidase polypeptide.

Examples of suitable promoting sequences for use with yeast hosts include the promoters for 3-phosphoglycerate kinase (Hitzeman et al., J. Biol. Chem., 255:2073 (1980)) or other glycolytic enzymes (Hess et al., J. Adv. Enzyme Reg., 7:149 (1968); Holland, Biochemistry, 17:4900 (1978)), such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

Expression vectors used in eukaryotic host cells (e.g. yeast, fungi, insect, plant) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding an engineered GH3 beta-glucosidase as described herein.

### Purification of an Engineered GH3 Beta-Glucosidase

In general, an engineered GH3 beta-glucosidase protein, such as the engineered Bgl1 herein, produced in cell culture is secreted into the medium and may be purified or isolated, e.g., by removing unwanted components from the cell culture medium. However, in some cases, such a variant protein may be produced in a cellular form necessitating recovery from a cell lysate. In such cases the variant GH3 beta-glucosidase protein is purified from the cells in which it was produced using techniques routinely employed by those of skill in the art. Examples include, but are not limited to, affinity chromatography (Tilbeurgh et al., FEBS Lett. 16:215, 1984), ion-exchange chromatographic methods (Goyal et al., Bioresource Technol. 36:37-50, 1991; Fliess et al., Eur. J. Appl. Microbiol. Biotechnol. 17:314-318, 1983; Bhikhabhai et al., J. Appl. Biochem. 6:336-345, 1984; Ellouz et al., J. Chromatography 396:307-317, 1987), including ion-exchange using materials with high resolution power (Medve et al., J. Chromatography A 808:153-165, 1998), hydrophobic interaction chromatography (Tomaz and Queiroz, J. Chromatography A 865:123-128, 1999), and two-phase partitioning (Brumbauer, et al., Bioseparation 7:287-295, 1999).

Typically, the variant engineered GH3 beta-glucosidase protein is fractionated to segregate proteins having selected properties, such as binding affinity to particular binding agents, e.g., antibodies or receptors; or which have a selected molecular weight range, or range of isoelectric points.

Once expression of a given variant GH3 beta-glucosidase protein is achieved, the protein thereby produced is purified from the cells or cell culture. Examples of procedures suitable for such purification include the following: antibody-affinity column chromatography, ion exchange chromatography; ethanol precipitation; reverse phase HPLC; chromatography on silica or on a cation-exchange resin such as DEAE; chromatofocusing; SDS-PAGE; ammonium sulfate precipitation; and gel filtration using, e.g., Sephadex G-75. Various methods of protein purification may be employed and such methods are known in the art and described e.g. in Deutscher, Methods in Enzymology, 182:779, 1990; Scopes, Methods Enzymol. 90:479-91, 1982. The purification step(s) selected will depend, e.g., on the nature of the production process used and the particular protein produced.

### Derivatives of Engineered GH3 Polypeptides

As described above, in addition to the engineered GH3 beta-glucosidase described herein, it is contemplated that GH3 enzyme derivatives can be prepared with altered amino acid sequences. In general, such GH3 enzyme derivatives would be capable of conferring, as a parent engineered GH3 beta-glucosidase, to a cellulase and/or hemicellulase mixture or composition either one or both of an improved capacity to hydrolyze a lignocellulosic biomass substrate. Such derivatives may be made, for example, to improve expression in a particular host, improve secretion (e.g., by altering the signal sequence), to introduce epitope tags or other sequences that can facilitate the purification and/or isolation of such an engineered polypeptides. In some embodiments, derivatives may confer more capacity to hydrolyze a lignocellulosic biomass substrate to a cellulase and/or hemicellulase mixture or composition, as compared to the parent engineered GH3 beta-glucosidase polypeptide.

GH3 beta-glucosidase derivatives can be prepared by introducing appropriate nucleotide changes into the engineered GH3 beta-glucosidase-encoding DNA, or by synthesis of the desired engineered GH3 beta-glucosidase polypeptides. Those skilled in the art will appreciate that amino acid changes may alter post-translational processes of these polypeptides, such as changing the number or position of glycosylation sites.

Derivatives of the engineered GH3 beta-glucosidase polypeptide or of various domains of the polypeptides described herein can be made, for example, using any of the techniques and guidelines for conservative and non-conservative mutations set forth, for instance, in U.S. Pat. No. 5,364,934. Sequence variations may be a substitution, deletion or insertion of one or more codons encoding the engineered GH3 beta-glucosidase polypeptide that results in a change in the amino acid sequence of the polypeptide as compared with the parent sequence. Optionally, the sequence variation is by substitution of at least one amino acid with any other amino acid in one or more of the domains of the engineered GH3 beta-glucosidase polypeptide.

Guidance in determining which amino acid residue may be inserted, substituted or deleted without adversely affecting the desired GH3 beta-glucosidase and/or beta-xylosidase activity may be found by comparing the sequence of the polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of 1 to 5 amino acids. The variation allowed may be determined by systematically making insertions, deletions or substitutions of amino acids in the sequence and testing the resulting derivatives for functional activity using techniques known in the art.

The sequence variations can be made using methods known in the art such as oligonucleotide-mediated (site-directed) mutagenesis, alanine scanning, and PCR mutagenesis. Site-directed mutagenesis (Carter et al., Nucl. Acids Res., 13:4331 (1986); Zoller et al., Nucl. Acids Res., 10:6487 (1987)), cassette mutagenesis (Wells et al., Gene, 34:315 (1985)), restriction selection mutagenesis (Wells et al., Philos. Trans. R. Soc. London SerA, 317:415 (1986)) or other known techniques can be performed on the cloned DNA to produce the engineered GH3 beta-xylosidase or beta-glucosidase encoding DNA with a variant sequence.

Scanning amino acid analysis can also be employed to identify one or more amino acids along a contiguous sequence. Among the scanning amino acids the can be employed are relatively small, neutral amino acids. Such amino acids include alanine, glycine, serine, and cysteine. Alanine is often used as a scanning amino acid among this group because it eliminates the side-chain beyond the beta-carbon and is less likely to alter the main-chain conformation of the derivative. Alanine is also often used because it is the most common amino acid. Further, it is frequently found in both buried and exposed positions (Creighton, The Proteins, (W.H. Freeman & Co., N.Y.); Chothia, J. Mol. Biol., 150:1 (1976)). If alanine substitution does not yield adequate amounts of derivative, an isosteric amino acid can be used.

### Engineered GH3 Beta-Glucosidase Antibodies

The present compositions and methods further provide anti GH3 beta-glucosidase, or anti-GH3 multifunctional beta-glucosidase/beta-xylosidase antibodies. Exemplary antibodies include polyclonal and monoclonal antibodies, including chimeric and humanized antibodies.

The anti-GH3 beta-glucosidase antibodies of the present compositions and methods may include polyclonal antibodies. Any convenient method for generating and preparing polyclonal and/or monoclonal antibodies may be employed, a number of which are known to those ordinarily skilled in the art.

Anti-GH3 beta-glucosidase antibodies of the present disclosure may also be generated using recombinant DNA methods, such as those described in U.S. Pat. No. 4,816,567.

The antibodies may be monovalent antibodies, which may be generated by recombinant methods or by the digestion of antibodies to produce fragments thereof, particularly, Fab fragments.

### Cell Culture Media

Generally, the microorganism is cultivated in a cell culture medium suitable for production of the engineered GH3 beta-glucosidase polypeptides described herein. The cultivation takes place in a suitable nutrient medium comprising carbon and nitrogen sources and inorganic salts, using procedures and variations known in the art. Suitable culture media, temperature ranges and other conditions for growth and cellulase production are known in the art. As a non-limiting example, a typical temperature range for the production of cellulases by Trichoderma reesei is 24° C. to 37° C., for example, between 25° C. and 30° C.

### Cell Culture Conditions

Materials and methods suitable for the maintenance and growth of fungal cultures are well known in the art. In some aspects, the cells are cultured in a culture medium under conditions permitting the expression of one or more engineered GH3 beta-glucosidase polypeptides encoded by a nucleic acid inserted into the host cells. Standard cell culture conditions can be used to culture the cells. In some aspects, cells are grown and maintained at an appropriate temperature, gas mixture, and pH. In some aspects, cells are grown at in an appropriate cell medium.

### Compositions Comprising an Engineered GH3 Beta-Glucosidase Polypeptide

The present disclosure provides engineered enzyme compositions (e.g., cellulase compositions) or fermentation broths enriched with an engineered GH3 beta-glucosidase polypeptide. In some aspects, the composition is a cellulase composition. The cellulase composition can be, e.g., a filamentous fungal cellulase composition, such as a Trichoderma cellulase composition. The cellulase composition can be, in some embodiments, an admixture or physical mixture, of various cellulases originating from different microorganisms; or it can be one that is the culture broth of a single engineered microbe co-expressing the cellulase genes; or it can be one that is the admixture of one or more individually/separately obtained cellulases with a mixture that is the culture broth of an engineered microbe co-expressing one or more cellulase genes.

In some aspects, the composition is a cell comprising one or more nucleic acids encoding one or more cellulase polypeptides. In some aspects, the composition is a fermentation broth comprising cellulase activity, wherein the broth is capable of converting greater than about 50% by weight of the cellulose present in a biomass sample into sugars. The term “fermentation broth” and “whole broth” as used herein refers to an enzyme preparation produced by fermentation of an engineered microorganism that undergoes no or minimal recovery and/or purification subsequent to fermentation. The fermentation broth can be a fermentation broth of a filamentous fungus, for example, a Trichoderma, Humicola, Fusarium, Aspergillus, Neurospora, Penicillium, Cephalosporium, Achlya, Podospora, Endothia, Mucor, Cochliobolus, Pyricularia, Myceliophthora or Chrysosporium fermentation broth. In particular, the fermentation broth can be, for example, one of Trichoderma sp. such as a Trichoderma reesei, or Penicillium sp., such as a Penicillium funiculosum. The fermentation broth can also suitably be a cell-free fermentation broth. In one aspect, any of the cellulase, cell, or fermentation broth compositions of the present invention can further comprise one or more hemicellulases.

In some aspects, the whole broth composition is expressed in T. reesei or an engineered strain thereof. In some aspects the whole broth is expressed in an integrated strain of T. reesei wherein a number of cellulases including an engineered GH3 beta-glucosidase polypeptide has been integrated into the genome of the T. reesei host cell. In some aspects, one or more components of the polypeptides expressed in the integrated T. reesei strain (e.g., a native beta-glucosidase, or a native beta-xylosidase) have been deleted.

In some aspects, the whole broth composition is expressed in A. niger or an engineered strain thereof.

Alternatively, the engineered GH3 beta-glucosidase polypeptide can be expressed intracellularly. Optionally, after intracellular expression of the enzyme variants, or secretion into the periplasmic space using signal sequences such as those mentioned above, a permeabilization or lysis step can be used to release the engineered GH3 beta-glucosidase polypeptide into the supernatant. The disruption of the membrane barrier is effected by the use of mechanical means such as ultrasonic waves, pressure treatment (French press), cavitation, or by the use of membrane-digesting enzymes such as lysozyme or enzyme mixtures. A variation of this embodiment includes the expression of an engineered GH3 beta-glucosidase polypeptide in an ethanologen microbe intracellularly. For example, a cellobiose transporter can be introduced through genetic engineering into the same ethanologen microbe such that cellobiose resulting from the hydrolysis of a lignocellulosic biomass can be transported into the ethanologen organism, and can therein be hydrolyzed and turned into D-glucose, which can in turn be metabolized by the ethanologen.

In some aspects, the polynucleotides encoding the engineered GH3 beta-glucosidase polypeptide are expressed using a suitable cell-free expression system. In cell-free systems, the polynucleotide of interest is typically transcribed with the assistance of a promoter, but ligation to form a circular expression vector is optional. In some embodiments, RNA is exogenously added or generated without transcription and translated in cell-free systems.

In certain embodiments, the enzyme composition comprising the engineered GH3 beta-glucosidase polypeptide as described herein may be a formulated enzyme mixture product. The formulated product may be one that is a liquid, or a gel, or a solid (e.g., a pellet, a granule, a particle, etc) or one that is a mixture, a suspension, a multi-compartment packages comprising a liquid, a suspension, a gel, a solid, or a combination thereof.

### Uses of Engineered GH3 Beta-Glucosidase Polypeptides and Compositions Comprising Such Polypeptides to Hydrolyze a Lignocellulosic Biomass Substrate

In some aspects, provided herein are methods for converting lignocellulosic biomass to sugars, the method comprising contacting the biomass substrate with a composition disclosed herein comprising an engineered GH3 beta-glucosidase polypeptide/variant in an amount effective to convert the biomass substrate to fermentable sugars.

In some aspects, the method further comprises pretreating the biomass with acid and/or base and/or mechanical or other physical means In some aspects the acid comprises phosphoric acid. In some aspects, the base comprises sodium hydroxide or ammonia. In some aspects, the mechanical means may include, for example, pulling, pressing, crushing, grinding, and other means of physically breaking down the lignocellulosic biomass into smaller physical forms. Other physical means may also include, for example, using steam or other pressurized fume or vapor to “loosen” the lignocellulosic biomass in order to increase accessibility by the enzymes to the cellulose and hemicellulose. In certain embodiments, the method of pretreatment may also involve enzymes that are capable of breaking down the lignin of the lignocellulosic biomass substrate, such that the accessibility of the enzymes of the biomass hydrolyzing enzyme composition to the cellulose and the hemicelluloses of the biomass is increased.

### Biomass

The disclosure provides methods and processes for biomass saccharification, using the enzyme compositions of the disclosure, comprising an engineered GH3 beta-xylosidase polypeptide as provided herein. The term “biomass,” as used herein, refers to any composition comprising cellulose and/or hemicellulose (optionally also lignin in lignocellulosic biomass materials). As used herein, biomass includes, without limitation, seeds, grains, tubers, plant waste (such as, for example, empty fruit bunches of the palm trees, or palm fibre wastes) or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), wood (including, e.g., wood chips, processing waste), paper, pulp, and recycled paper (including, e.g., newspaper, printer paper, and the like). Other biomass materials include, without limitation, potatoes, soybean (e.g., rapeseed), barley, rye, oats, wheat, beets, and sugar cane bagasse.

The disclosure therefore provides methods of saccharification comprising contacting a composition comprising a biomass material, for example, a material comprising xylan, hemicellulose, cellulose, and/or a fermentable sugar, with an engineered GH3 beta-glucosidase polypeptide of the disclosure, or an engineered GH3 beta-glucosidase polypeptide encoded by a nucleic acid or polynucleotide of the disclosure, or any one of the cellulase or non-naturally occurring hemicellulase compositions comprising an engineered GH3 beta-glucosidase polypeptide, or products of manufacture of the disclosure.

The saccharified biomass (e.g., lignocellulosic material processed by enzymes of the disclosure) can be made into a number of bio-based products, via processes such as, e.g., microbial fermentation and/or chemical synthesis. As used herein, “microbial fermentation” refers to a process of growing and harvesting fermenting microorganisms under suitable conditions. The fermenting microorganism can be any microorganism suitable for use in a desired fermentation process for the production of bio-based products. Suitable fermenting microorganisms include, without limitation, filamentous fungi, yeast, and bacteria. The saccharified biomass can, for example, be made it into a fuel (e.g., a biofuel such as a bioethanol, biobutanol, biomethanol, a biopropanol, a biodiesel, a jet fuel, or the like) via fermentation and/or chemical synthesis. The saccharified biomass can, for example, also be made into a commodity chemical (e.g., ascorbic acid, isoprene, 1,3-propanediol), lipids, amino acids, polypeptides, and enzymes, via fermentation and/or chemical synthesis.

### Pretreatment

Prior to saccharification or enzymatic hydrolysis and/or fermentation of the fermentable sugars resulting from the saccharification, biomass (e.g., lignocellulosic material) is preferably subject to one or more pretreatment step(s) in order to render xylan, hemicellulose, cellulose and/or lignin material more accessible or susceptible to the enzymes in the enzymatic composition (for example, the enzymatic composition of the present invention comprising an engineered GH3 beta-glucosidase polypeptide as provided herein) and thus more amenable to hydrolysis by the enzyme(s) and/or the enzyme compositions.

In some aspects, a suitable pretreatment method may involve subjecting biomass material to a catalyst comprising a dilute solution of a strong acid and a metal salt in a reactor. The biomass material can, e.g., be a raw material or a dried material. This pretreatment can lower the activation energy, or the temperature, of cellulose hydrolysis, ultimately allowing higher yields of fermentable sugars. See, e.g., U.S. Pat. Nos. 6,660,506; 6,423,145.

In some aspects, a suitable pretreatment method may involve subjecting the biomass material to a first hydrolysis step in an aqueous medium at a temperature and a pressure chosen to effectuate primarily depolymerization of hemicellulose without achieving significant depolymerization of cellulose into glucose. This step yields a slurry in which the liquid aqueous phase contains dissolved monosaccharides resulting from depolymerization of hemicellulose, and a solid phase containing cellulose and lignin. The slurry is then subject to a second hydrolysis step under conditions that allow a major portion of the cellulose to be depolymerized, yielding a liquid aqueous phase containing dissolved/soluble depolymerization products of cellulose. See, e.g., U.S. Pat. No. 5,536,325.

In further aspects, a suitable pretreatment method may involve processing a biomass material by one or more stages of dilute acid hydrolysis using about 0.4% to about 2% of a strong acid; followed by treating the unreacted solid lignocellulosic component of the acid hydrolyzed material with alkaline delignification. See, e.g., U.S. Pat. No. 6,409,841.

In yet further aspects, a suitable pretreatment method may involve pre-hydrolyzing biomass (e.g., lignocellulosic materials) in a pre-hydrolysis reactor; adding an acidic liquid to the solid lignocellulosic material to make a mixture; heating the mixture to reaction temperature; maintaining reaction temperature for a period of time sufficient to fractionate the lignocellulosic material into a solubilized portion containing at least about 20% of the lignin from the lignocellulosic material, and a solid fraction containing cellulose; separating the solubilized portion from the solid fraction, and removing the solubilized portion while at or near reaction temperature; and recovering the solubilized portion. The cellulose in the solid fraction is rendered more amenable to enzymatic digestion. See, e.g., U.S. Pat. No. 5,705,369. In a variation of this aspect, the pre-hydrolyzing can alternatively or further involves pre-hydrolysis using enzymes that are, for example, capable of breaking down the lignin of the lignocellulosic biomass material.

In yet further aspects, suitable pretreatments may involve the use of hydrogen peroxide H₂O₂. See Gould, 1984, Biotech, and Bioengr. 26:46-52.

In other aspects, pretreatment can also comprise contacting a biomass material with stoichiometric amounts of sodium hydroxide and ammonium hydroxide at a very low concentration. See Teixeira et al., (1999), Appl. Biochem. and Biotech. 77-79:19-34.

In some embodiments, pretreatment can comprise contacting a lignocellulose with a chemical (e.g., a base, such as sodium carbonate or potassium hydroxide) at a pH of about 9 to about 14 at moderate temperature, pressure, and pH. See Published International Application WO2004/081185. Ammonia is used, for example, in a preferred pretreatment method. Such a pretreatment method comprises subjecting a biomass material to low ammonia concentration under conditions of high solids. See, e.g., U.S. Patent Publication No. 20070031918 and Published International Application WO 06110901.

### Saccharification Process

In some embodiments, provided herein is a saccharification process comprising treating biomass with an enzyme composition comprising an engineered GH3 beta-glucosidase polypeptide, wherein the engineered GH3 beta-glucosidase has not only beta-glucosidase activity but also acquires beta-xylosidase activity, wherein the process results in at least about 50 wt. % (e.g., at least about 55 wt. %, 60 wt. %, 65 wt. %, 70 wt. %, 75 wt. %, or 80 wt. %) conversion of biomass to fermentable sugars. In some aspects, the biomass comprises lignin. In some aspects the biomass comprises cellulose. In some aspects the biomass comprises hemicellulose. In some aspects, the biomass comprising cellulose further comprises one or more of xylan, galactan, or arabinan. In some aspects, the biomass may be, without limitation, seeds, grains, tubers, plant waste (e.g., empty fruit bunch from palm trees, or palm fibre waste) or byproducts of food processing or industrial processing (e.g., stalks), corn (including, e.g., cobs, stover, and the like), grasses (including, e.g., Indian grass, such as Sorghastrum nutans; or, switchgrass, e.g., Panicum species, such as Panicum virgatum), perennial canes (e.g., giant reeds), wood (including, e.g., wood chips, processing waste), paper, pulp, and recycled paper (including, e.g., newspaper, printer paper, and the like), potatoes, soybean (e.g., rapeseed), barley, rye, oats, wheat, beets, and sugar cane bagasse. In some aspects, the material comprising biomass is subject to one or more pretreatment methods/steps prior to treatment with the polypeptide. In some aspects, the saccharification or enzymatic hydrolysis further comprises treating the biomass with an enzyme composition comprising an engineered GH3 beta-glucosidase polypeptide of the invention. The enzyme composition may, for example, comprise one or more other cellulases, in addition to the engineered GH3 beta-glucosidase polypeptide. Alternatively, the enzyme composition may comprise one or more other hemicellulases. In certain embodiments, the enzyme composition comprises an engineered GH3 beta-glucosidase polypeptide of the invention, one or more other cellulases, one or more hemicellulases. In some embodiments, the enzyme composition is a whole broth composition.

In certain embodiments, provided is a saccharification process comprising treating a lignocellulosic biomass material with a composition comprising a polypeptide, wherein the polypeptide has at least about 70% (e.g., at least about 70%, 75%, 80%, 85%, 90%, 91%, 92%, 93%, 94%, 95%, 96%, 97%, 98%, 99%) sequence identity to SEQ ID NO:2, or SEQ ID NO:3, and one or more substitutions at positions 43, 237, and 255, with the numbering referencing SEQ ID NO:3, and wherein the process results in at least about 50% (e.g., at least about 55%, 60%, 65%, 70%, 75%, 80%, 85%, or 90%) by weight conversion of biomass to fermentable sugars. In some aspects, lignocellulosic biomass material has been subject to one or more pretreatment methods/steps as described herein.

Other aspects and embodiments of the present compositions and methods will be apparent from the foregoing description and following examples.

### EXAMPLES

The following examples are put forth so as to provide those of ordinary skill in the art with a complete disclosure and description of how to make and use the present compositions and methods, and are not intended to limit the scope of what the inventors regard as their inventive compositions and methods nor are they intended to represent that the experiments below are all or the only experiments performed. Efforts have been made to ensure accuracy with respect to numbers used (e.g. amounts, temperature, etc.) but some experimental errors and deviations should be accounted for.

### Example 1

### Purification of Trichoderma reesei Beta-Glucosidase I (Bgl1)

The native gene encoding Trichoderma reesei beta-glucosidase I (Bgl1) (UniProt Q12715) was overexpressed in a Trichoderma reesei strain lacking four genes coding for cellulases (cbh1, cbh2, egl1, egl2). The target genes were cloned into the pTrex3G vector (amdS^{R}, amp^{R}, P_{cbh1}), see, e.g., published application US 20070128690, and used to transform the above Trichoderma reesei strain. Transformants were picked from Vogel's minimal medium plates (see, Vogel H. J., (1956) A convenient growth medium for Neurospora (medium N), Microbial Genetics Bulletin, 13:42-43) containing acetamide, after 7 days of growth at 37° C. Those transformants were grown up in Vogel's minimal medium with a mixture of glucose and sophorose as a carbon source. The overexpressed proteins appeared as dominant proteins in culture supernatants, in that the Trichoderma reesei Bgl1 was approximately 80% pure as judged by visualization of the SDS-PAGE.

Ten (10) mL of cell culture filtrate from production run was diluted in 90 mL 25 mM Na-acetate buffer, at pH4. After mixing, the sample was incubated at 37° C. for 30 min. The sample was then desalted using a Sephadex G-25M column (GE Healthcare, Piscataway, N.J., USA), which had been previously equilibrated with the Na-acetate buffer, at pH 4. Volumes of 2.5 mL each of the sample were loaded to the column and eluted with 3.5 mL acetate buffer. Fractions containing protein were pooled and concentrated to a volume of 10 mL using a centrifugal concentrator with a 10 kD cutoff (Vivascience, Littleton, Mass., USA).

The resulting sample was then loaded onto a high load 26/60 Superdex 200 column (GE Healthcare, Piscataway, N.J., USA), which had been previously equilibrated with the Na-acetate buffer, at pH 4.0, containing also 100 mM NaCl. Protein was eluted with the same buffer and protein-containing fractions were checked on SDS-PAGE gel for purity. Fractions with visually pure Trichoderma reesei Bgl1 were pooled and stored at 4° C. Enzyme purity was also confirmed by IEF analysis.

### Example 2

### Crystallization, Data Collection, Structural Determination and Refinement of Trichoderma reesei Bgl1

The purified Bgl1 as described in Example 1 above was concentrated to 3.9 mg/mL in a buffer containing 25 mM NaAc (pH 4), and 100 mM NaCl. Bgl1 crystals were obtained using the hanging-drop vapor diffusion method at 20° C.

More specifically, the drops were prepared by mixing equal volume of protein sample and crystallization solution containing 0.1 M sodium formate, at pH 7.0, and 10-20% PEG 3350. To produce Bgl1-glucose or Bgl1 (1-thio-beta-D-glucosyldisulfanyl)1-thio-beta-D-glucose (Bgl1-GSSG) complex crystals, Bgl1 crystals were soaked into the crystallization solution containing an addition of 50 mM glucose or 20 mM 4-thio-cellobiose for a period of 10 min before they were frozen.

Prior to data collection, crystals were frozen in liquid nitrogen, after the crystallization solution with 20% glycerol had been added as a cryo-protectant. Glucose was also added to the cryo-protectant to a final concentration of 50 mM for the Bgl1-glucose crystals. Likewise, 4-thiocellobiose was added to the cryo-protectant to a final concentration of 10 mM for the Bgl1-GSSG crystals.

Crystallographic coordinates for each of Bgl1, Bgl1-glucose, and Bgl1-GSSG were collected on beam line I-911-5 at MAX-lab (Lund, Sweden), at ESRF beam line BM-14 (Grenoble, France), and beam line I-911-3 at MAX-lab (Lund, Sweden), respectively, from single crystals at 100 K. The X-ray diffraction data were processed using the X-ray data integration program Mosflm (see, Leslie, A.g., (2006) The Integration of macromolecular diffraction data, Acta Crystallogr. D. Biol. Crystallogr. 62:48-57) and scaled using the scaling program Scala (see, Evans, P., (2006) Scaling and assessment of data quality, Acta Crystallogr. D. Biol. Crystallogr. 62:72-82) in the CCP4i program package (see, High-throughput structure determination. Proceedings of the 2002 CCP4 (Collaborative Computational Project in Macromolecular Crystallography) study weekend. January, 2002. York, United Kingdom. (2002) Acta Crystallogr. D. Biol. Crystallogr. 58:1897-970; see also, Dodson, E. J., et al., (1997) Collaborative Computational Project, number 4: providing programs for protein crystallography, Methods Enzymol. 277:620-33) In the case of Bgl1-glucose complex, the data were processed and scaled with XDS package (see, Kabsch W., (2010) Xds. Acta. Crystallogr. D. Biol. Crystallogr. 66:125-32).

Details of data collection and processing are presented in Table 2.

TABLE 2

| 1). Data collection and processing | T. Reesei Bgl1 | Bgl1-glucose |
|---|---|---|
| PDB code | 3zz1 | 3zyz |
| Beamline^{a} | I911-5 | BM14 |
| Wavelength (Å) | 0.90817 | 0.95373 |
| No. Of images | 175 | 120 |
| Oscillation range (°) | 0.6 | 1.0 |
| Space group | P2₁2₁2₁ | P2₁2₁2₁ |
| Cell dimensions (a, b, c) | 55.1 82.4 136.7 | 55.1 82.9 136.8 |
| Cell angles (α, β, χ) | 90, 90, 90 | 90, 90, 90 |
| Resolution range (Å) | 29.7-2.1 | 29.7-2.1 |
| Resolution range outer shell | 2.21-2.10 | 2.21-2.10 |
| No. Of observed reflections | 152209 | 217624 |
| No. Of unique reflections | 36726 | 37117 |
| Average multiplicity | 4.1(3.9) | 5.9(5.4) |
| Completeness (%)^{b} | 99.0 (95.0) | 99.8(99.5) |
| R_{merge} (%)^{c} | 14.0 (38.1) | 15.0(49.5) |
| I/σ(I) | 8.1 (3.1) | 9.8(3.2) |
| 2). Refinement | Cel3A | Cel3A-GC |
| Resolution used in refinement (Å) | 30.0-2.10 | 30.0-2.10 |
| No. of reflections | 34896 | 35114 |
| R work(%) | 17.4 | 17.2 |
| Rfree (%) | 22.3 | 22.2 |
| No. of residues in protein | 713/713 | 713/713 |
| No. of residues in the a.u with | 9 | 12 |
| alternate conformations | | |
| No. of water molecules | 690 | 611 |
| Average atomic B-factor (Å2) | | |
| overall | 15.1 | 14.4 |
| protein | 14.1 | 13.6 |
| Rmsd for bond lengths (Å)d | 0.007 | 0.009 |
| Rmsd for bond angles (deg)d | 1.024 | 1.190 |
| Ramachandran outliers (%) | | |
| favored | 94.16 | 95.41 |
| allowed | 5.13 | 3.87 |
| outlier | 0.71 | 0.72 |

The wild type T. reesei Bgl1, Bgl1-glucose, and Bgl1-CSSG complex crystals were found to belong to the orthorhombic space group P212121, with approximate unit-cell parameters of: a=55.06, b=82.40, and c=136.7.

### Example 3

### Preparation and Purification of Trichoderma reesei Beta-Xylosidase Xyl3A

The gene encoding for Trichoderma reesei (or H. jecorina) Xyl3A (GenBank accession code CAA93248.1, UniProt accession code Q92458) (see, Margolles-Clark, E., et al., (1996) Cloning of Genes Encoding alpha-L-arabinofuranosidase and beta-xylosidase from Trichoderma reesei by expression in Saccharomyces cerevisiae. App. Environ. Microbiol. 62(10): 3840-46) has been sequenced from a H. jecorina QM6a cDNA library as described in Foreman P K et al. (2003) Transcriptional regulation of biomass-degrading enzymes in the filamentous fungus Trichoderma reese, J Biol Chem. August 22; 278 (34):31988-97. The open reading frame (ORF) of the gene was amplified from H. jecorina QM6a genomic DNA by PCR using the primers:

bxl1F:
(SEQ ID NO: 6)
5′-CACCATGGTGAATAACGCAGCTC-3′;
and
bxl1R:
(SEQ ID NO: 7)
5′-TTATGCGTCAGGTGTAGCATC-3′,

and inserted into pENTR/D-TOPO (Invitrogen Corp., Carlsbad, Calif.) using the TOPO cloning reaction.

Subsequently, the open reading frame of bxl1 was transferred to pTrex3g, using the LR clonase reaction (Invitrogen) to create the expression vector pTrex3Gbxl1 with the bxl1 ORF flanked by the cbh1 promoter and terminator.

The pTrex3g vector is based on the E. coli plasmid pSL1180 (Pharmacia Inc., Piscataway, N.J.). It was designed as a Gateway destination vector (Hartley, Temple et al. 2000; Walhout, Temple et al. 2000) to allow insertion using Gateway technology (Invitrogen) of any desired ORF between the promoter and terminator regions of the H. jecorina cbh1 gene. It also contains the Aspergillus nidulans amdS gene, with its native promoter and terminator, as selectable marker for transformation.

A Trichoderma reesei host strain was derived from strain RL-P37 (Sheir-Neiss and Montenecourt 1984) by sequential deletion of the genes encoding the four major secreted cellulases (cbh1, cbh2, egl1 and egl2). Transformation with pTrex3gbxl1 was performed using a Bio-Rad Laboratories, Inc. (Hercules, Calif.) model PDS-1000/He biolistic particle delivery system according to the manufacturers instructions. Transformants were selected on solid medium containing acetamide as the sole nitrogen source.

For Xyl3A production, transformants were cultured in a liquid minimal medium containing lactose as carbon source as described previously (Ilmen, M., et al., (1997) Appl Environ Microbiol 63:1298-1306), except that 100 mM piperazine-N, N-bis (3-propanesulfonic acid) (Calbiochem) was included to maintain the pH at 5.5. Culture supernatants were analyzed by SDS-PAGE under reducing conditions and the strain that produced the highest level of a band with apparent molecular weight of approximately 90 kDa was selected for further analysis and grown at 25° C., 200 rpm in a batch-fed process, using a minimal fermentation medium of 0.8 L containing 5% glucose, incubated with 1.5 mL of spore suspension, essentially as described in Ilmen et al. (1997) Regulation of cellulase gene expression in the filamentous fungus Trichoderma reesei, Appl. Environ. Microbiol. April 63(4): 1298-306.

After 48 hours, the culture was transferred to 6.2 L of the same media in a 14 L fermenter (Biolafitte, N.J.). One (1) hour after the glucose was exhausted, a 25% (w/w) lactose feed was started in a carbon limiting fashion so as to prevent its accumulation. The pH during fermentation was maintained in the range of pH 4.5-5.5. Xyl3A was expressed at several grams per litre, constituting more than 50% of the total secreted protein, as judged by SDS-PAGE. The supernatant was concentrated to 168 g total protein/L by ultrafiltration at 4° C.

### Example 4

### Crystallization, Data Collection, Structural Determination and Refinement of Trichoderma reesei Xyl3A

The Xyl3A protein was stored at 4° C. in a stock solution containing 149 mg/mL protein, 13% sorbitol and 0.125% Sodium benzoate, in culture medium. The protein stock solution was diluted to 10 mg/mL by adding 0.1 M sodium acetate buffer pH 4.5 just prior to crystallisation.

Initial screening for crystallization conditions for Xyl3A were carried out using JCSG+ (Qiagen), PEG Ion HT and HCS I+II screens and the vapour diffusion crystallization method using sitting drops in Greiner Low profile 96 well plates. See, Manuela Benvenuti & Stephano Magnani (2007) Crystallization of soluble proteins in vapor diffusion for x-ray crystallography, Nature Protocols, 2: 1633-1651 (2007). Crystallization drops were prepared by mixing the protein solution with an equal volume of well solution. Crystals commonly started to appear after a few hours incubation and grew in size within 1 to 3 days in condition E6 in JCSG+, C2 in Peg Ion HT and D9 in HCS I+II at 20 degrees. Optimization of crystal condition C2 in PEG Ion HT was performed using Hampton additive screen.

Crystals for data collection were obtained by the hanging drop vapour diffusion method. For multiple anomalous dispersion (MAD) data collection, crystals were obtained by mixing 2 μL of protein solution, 2 μL of well solution A (15% PEG 3350, 0.2M zinc acetate, and 0.1M Tris-Cl pH 8.5) and 0.5 μL of 0.1 M magnesium chloride hexahydrate. For high-resolution data collection, crystals were obtained by mixing equal volumes of Xyl3A protein solution, 15 mg/mL, with the well solution B (22% PEG 3350, 0.2 M zinc acetate and 0.1 M Tris-Cl pH 8.5). Crystals for ligand data collection were obtained in PACT screen (Qiagen etc) condition C4 (0.1 M PCB pH 7.0 and 25% PEG1500). Soaking of xylose and 4-thioxylobiose to the crystals was done by a one-hour incubation of crystals in 0.095M PCB, pH 7.0 and 33% PEG1500 with either 10 mM xylose (SIGMA etc) or 14 mM 4-thioxylobiose, which was custom synthesized using the protols as described in Jacques Defayea et al. (1985), Induction of d-xylan-degrading enzymes in Trichoderma lignorum by nonmetabolizable inducers. A synthesis of 4-thioxylobiose. Carbohydrate Research, 139, 15 Jun. 1985, Pages 123-132.

Prior to data collection, crystals were passed through a cryoprotectant solution containing 30% PEG 3350 and 10% glycerol and flash frozen in liquid nitrogen prior to storage and transport to the synchrotron X-ray source.

The MAD and the high-resolution native datasets were collected at beamline 1911-3 at MAX-lab, Lund, Sweden. The datasets of crystals soaked with xylose (to 2.4 Å resolution) and 4-thioxylosbiose (to 2.1 Å resolution) were collected at the beamline 1911-5. All data were processed using the data integration program Mosflm (Leslie 2006) and scaled using Scala in the CCP4 Software suite (Collaborative Computational Project Number 4. 1994).

Details of data collection and processing are presented in Table 3:

TABLE 3

| 1). Data collection and | | |
|---|---|---|
| processing | Xyl3A | Xyl3A-thioxylobiose |
| PDB code | Not yet | Not yet |
| | deposited | deposited |
| Beamline^{a} | I911-3 | I911-2 |
| Wavelength (Å) | 0.99 | 1.03796 |
| No. Of images | 175 | 201 |
| Oscillation range (°) | 0.8 | 0.5 |
| Space group | P2₁2₁2 | P2₁2₁2 |
| Cell dimensions (a, b, c) | 99.9 203.7 82.1 | 100.2 202.4 82.4 |
| Cell angles (α, β, χ) | | |
| Resolution range (Å) | 26.6-1.8 | 29.8-2.1 |
| Resolution range outer shell | 1.90-1.80 | 2.29-2.10 |
| No. Of observed reflections | 146943 | 408594 |
| No. Of unique reflections | 139579 | 93526 |
| Average multiplicity | 5.35 | 4.15 |
| Completeness (%)^{b} | 99(94.5) | 99.9(99.9) |
| R_{merge} (%)^{c} | 14(66) | 9(39) |
| I/σ(I) | 5.1(1.3) | 6.6(2.3) |
| 2). Refinement | Xyl3A | Xyl3A-thioxylobiose |
| Resolution used in refinement | 30-1.8 | 30-2.1 |
| (Å) | | |
| No. of reflections | 139579 | 63549 |
| R work(%) | 16.2 | 18.8 |
| Rfree (%) | 20.0 | 23.2 |
| No. of residues in protein | 766/767 | 766/767 |
| No. of residues in the a.u with | 50 | 7 |
| alternate conformations | | |
| No. of water molecules | 1983 | 632 |
| Average atomic B-factor (Å2) | | |
| overall | | |
| protein | | |
| Rmsd for bond lengths (Å)d | 0.011 | 0.006 |
| Rmsd for bond angles (deg)d | 1.293 | 1.144 |
| Ramachandran outliers (%) | | |
| favored | 98.1 | 97.7 |
| allowed | | |
| outlier | 1.9 | 2.3 |

MAD technique (Hendricksen Wash., et al. (1985) Direct phase determination based on anomalous scattering, Methods Enzymol. 115:41-55) was used for structure determination of Xyl3A to 2.1 Å resolution using the PHENIX software suite. See, Adam PDI., et al. (2002) PHENIX: building new software for automated crystallographic structure determination. Acta Crystallogr. D. Biol. Crystallogr. 58 (Pt. 11): 1948-54; Adam PDI., et al. (2011) The Phenix software for automated determination of macromolecular structure, Methods 55(1): 94-106. The position of 14 zinc atoms bound to the protein where found making it possible to calculate initial phases and perform density modification. The Autobuild function in PHENIX built more than 80% of the complete structure model including solvent. The high-resolution structure was solved by molecular replacement (MR) using the program Phaser with the structure model solved by MAD technique to a 2.1 Å resolution as a search model. Xylose-bound and 4-thioxylobiose-bound structure models were refined to 2.4 Å and 2.1 Å resolution, respectively, using the phases from the 1.8 Å structure model. See, McCoy (2007) Solving structures of protein complexes by molecular replacement with Phaser. Acta Crystallogr. D. Biol. Crystallogr. 63 (Pt. 1): 32-41; McCoy et al. (2007) Phaser crystallographic software, J. Appl. Crystallogr. 40 (Pt. 4): 658-674.

Structure refinement was performed using the program REFMACS and 5% of the data was excluded from the refinement for cross-validation and R_{free} calculations. See, Murshudov et al. (1997) Refinement of macromolecular structures by the maximum-likelihood method, Acta Crystallogr. D. Biol. Crystallogr. 53 (Pt. 3): 240-255; Brunger (1992), Free R. value: a novel statistical quantity for assessing the accuracy of crystal structures, Nature 355 (6359): 472-475). Throughout the refinement 2mFₒ-DF_{c} and mFₒ-DF_{c} sigma A weighted maps were generated and inspected so that the model could manually be built and adjusted in Coot. Pannu et al. (1996) Improved structure refinement through maximum likelihood, Acta Crystallogr. A52: 659-668; Emsley et al. (2004) Coot: model-building tools for molecular graphics, Acta. Crystallog. D. Biol. Crystallogr. 60 (Pt. 12, Pt. 2) 2126-2132). The statistics of refinement is shown in table 1b. Figures were rendered using the molecular visualization program PyMOL. See, DeLano (2002) The PyMOL Molecular Graphics System, Palo Alto, Calif. USA, Delano Scientific. The coordinates for the final structure models and structure-factors amplitudes for these have been deposited at the Protein Data Bank (PDB). See, Bernstein et al., (1977) The Protein Data Bank: a computer-based archival file for macromolecular structures. J. Mol. Biol. 112 (3): 535-542; Keller et al. (1998) Deposition of macromolecular structures, Acta. Crystallog. D. Biol. Crystallogr. 54 (Pt. 6 Pt. 1): 1105-1108; Sussman et al. (1998) Protein Data Bank (PDB): database of three-dimensional structural information of biological macromolecules, Acta. Crystallog. D. Biol. Crystallogr. 54 (Pt. 6 Pt. 1): 1078-1084).

### Example 5

### The Crystal Structures of Trichoderma reesei Bgl1 and Xyl3A

### Bgl1 Crystal Structure:

Trichoderma reesei expressed Bgl1 crystallized with one molecule in the asymmetric unit in space group P2₁, both apo (Bgl1-apo), glucose (Bgl1-glucose) forms. Both structures were solved to 2.1 Å. The crystallographic R-factors for the final structure models of the Bgl1 and Bgl1-glucose complex are 17.5% and 18.3%, respectively, while the R-free values are 22.2% and 22.8%, respectively. Other refinement statistics are provided in Table 2 (above).

The overall fold of Bgl1 is composed of three distinct domains (FIG. 1). Superposition of this structure with the structure of TnBgl3B structures gives an RMSD of 1.63 Å for 713 equivalent Cα positions, using the SSM algorithm. See, Pozzo et al. (2010) Structural and functional analyses of beta-glucosidase 3B from Thermotoga neapolitana: a thermostable three-domain representative of glycoside hydrolase 3, J. Mol. Biol. 397(3):724-739.

Domain 1 of Bgl1 encompasses residues 7 to 300. This domain is joined to Domain 2 with a 16 residues long linker (301-316). Domain 2, a five-stranded α/β sandwich, comprises residues 317 to 522 is followed by a third domain, Domain 3, which is composed of residues 580 to 714, and has a immunoglobulin type topology. The folds represented by Domain 1 and Domain 2 together are present in many GH3 β-glucosidases and the fold was first described for a barley Hordeum vulgare GH3 b-glucanase HvExo1 (Varghese, J. N., M. Hrmova, and G. B. Fincher, Three-dimensional structure of a barley beta-D-glucan exohydrolase, a family 3 glycosyl hydrolase. Structure, 1999. 7(2): p. 179-90.) While the Domain 1 of HvExo1 has a canonical TIM barrel fold, with an alternating repeat of eight α-helices and eight parallel β-strands α/β barrel, Domain 1 of Trichoderma reesei Bgl1 lacks three of the parallel β-strands and the two intervening α-helices. This was similarly reported for Bgl3 of Thermotoga neapolitana. Instead, the Bgl1 Domain 1 has 3 short anti-parallel β-strands, which together with five parallel β-strands and six α-helices form an incomplete or collapsed α/β barrel.

It was noted that the Domain 3 of Bgl1 is almost identical to Domain 3 of Bgl3 of Thermotoga neapolitana (TnBgl3B). The Bgl1 was found to have low RMSD value of 1.04 Å after superposition of the two domains over 113 equivalent Cα positions. Comparing the Domain 3's of Bgl1 vs. TnBgl3B, major differences were observed in the region where the β-strands Lys581-Thr592 and Val614-Ser624 of Bgl1 are connected. The two corresponding β-strands in TnBgl3B were observed to be connected with a short loop whereas in Bgl1, a notably larger structural insertion was observed Ala593-Asn613.

### Xyl3A Crystal Structures

The crystal structure of β-xylosidase Xyl3A from Hypocrea jecorina was determined at 1.8 Å resolution by X-ray crystallography, representing the first structure of a glycoside hydrolase (GH) family 3 enzyme primarily active on xylans.

The crystallization studies revealed that Xyl3A only crystallized with zinc present and the structure was initially solved with a 2.1 Å resolution dataset using the MAD technique with zinc as anomalous scatterer.

The original crystal form was P2₁2₁2₁, for which the MAD data set was collected on one crystal. The data was cut at 2.3 Å for the structure determination and the positions of 14 zinc atoms bound to the protein were identified by HYSS. See, Grosse-Kunstleve et al. (2003) Substructure search procedures for macromolecular structures, Acta Crystallog. D. Biol. Crystallogr. 59 (Pt. 11) 1966-1973. The score after calculating the initial phase from SOLVE was 63.9 and the map correlation coefficient was 0.65 after density modification using RESOLVE. See, Terwilliger et al. (1999) Automated MAD and MIR structure solution, Acta Crystallog. D. Biol. Crystallogr. 55 (Pt. 4): 849-861; Terwilliger (2000) Maximum-likelihood density modification, Acta. Crystallog. D. Biol. Crystallogr. 56 (Pt. 8): 965-972; Terwilliger (2003) Automated main-chain model building by template matching and iterative fragment extension, Acta. Crystallog. D. Biol. Crystallogr. 59 (Pt. 1): 38-44. The Autobuild function in PHENIX built more than 80% of the complete structure model including solvent.

Data for MAD phasing and structure determination is presented in Table 3 (above).

Improved crystals were obtained with a different crystal form, P2₁2₁2, for which a data set was collected that diffracted to 1.8 Å resolution. Two ligand datasets were also collected on the improved crystals soaked with xylose and 4-thioxylosbiose, respectively. The high-resolution structure was solved by molecular replacement using the initial structure model from MAD phasing as search model. In both crystal forms of Xyl3A, the asymmetric unit contained two enzyme molecules. The main difference between the two crystal forms appeared to be a different glycosylation pattern. Specifically, Xyl3A appeared to be a glycosylated 3-domain protein of 777 amino acid residues. In 4 structure models built based on the diffraction pattern, the electron density is well defined. Specifically, 11 residues at the C-terminus were not visible in the electron density map for any of the structure models built, and the density appeared ill-defined for 5 residues in the loop between residues 628 and 634 in one of the two Non-Crystallographic Symmetry molecules in all structure models. Using the method of Marshall, 1972, 10 asparagine residues in Asn-xaa-Thr/Ser sites were found to be N-glycosylated on each molecule. See, Marshall (1972) Glycoproteins, Ann. Rev. Biochem. 41:673-702.

FIG. 2 shows a cartoon representation of the Xyl3A domain structure and the NCS dimer of the 1.8 Å resolution structure model. Xyl3A has three distinct domains with the same domain architecture as reported for the bacterial GH3 β-glucosidase TnBgl3B and also similar to that of another fungal BglI from Kluyveromyces marxianus (KmBglI), although Xyl3A and TnBgl3B both lacks the PA14 domain present as an insert in domain 2 of KmBglI. See, Pozzo et al. (2010) Structural and functional analyses of beta-glucosidase 3B from Thermotoga neapolitana: a thermostable three-domain representative of glycoside hydrolase 3, J. Mol. Biol. 397 (3): 724-739; Yoshida et al. (2010) Role of a Pa14 domain in determining substrate specificity of a glycoside hydrolase family 3 beta-glucosidase from Kluyveromyces marxianus, Biochem. J. 431(1): 39-49.

Similar to other multi-domain GH3 enzymes, the active site of Xyl3A is located in the interface between domain 1 and 2 and has the same functional build up as has been reported for all other GH3 β-glucosidases with known three dimensional structure. Only two of the active site residues, the catalytic acid/base Glu492 and Tyr429, are located on domain 2. The nucleophile (Asp291) is located on domain 1 as are most of the other active site residues of Xyl3A: Pro15, Leu17 Glu89, Tyr152, Arg166, Lys206, His207, Arg221 and Tyr257. Lys206 and His207 form part of a conserved motif with cis-peptide bonds after Lys206 and the Phe208. See, Harvey et al (2000) Comparative modeling of the three-dimensional structure of family 3 glycoside hydrolases, Proteins 41(2): 257-69; Pozzo et al. (2010) Structural and functional analyses of beta-glucosidase 3B from Thermotoga neapolitana: a thermostable three-domain representative of glycoside hydrolase 3, J. Mol. Biol. 397 (3): 724-739. These cis-peptide bonds have been suggested to allow a correct side chain conformation for the substrate interaction by Lys206 and His207. See, Pozzo et al. (2010) Structural and functional analyses of beta-glucosidase 3B from Thermotoga neapolitana: a thermostable three-domain representative of glycoside hydrolase 3, J. Mol. Biol. 397 (3): 724-739. Except from Lys206, His207 and Asp291, remarkably few of the active site residues are conserved. Glu89, which form H-bond to OH-4 of a xylose residue in subsite −1, seem to be a conserved glutamate among fungal β-xylosidases. On the other hand, in most β-glucosidases and in all GH3 enzymes with known structure this residue is most commonly an aspartate.

The active site geometry is narrower in Xyl3A compared to both TnBgl3B and HvExo1. Residues Gln14, Pro15, Leu17 and Leu22 from the N-terminal region restrict the space for a xylose residue in the +1 subsite on one side. The backbone amide of Leu22 and the backbone carbonyl of Leu17 form a small water mediated hydrogen bond network with the O1 hydroxyl group of the +1 xylose residue in the 4-thioxylobiose complex with Xyl3A. Trp87 is located next to Leu22 and within van der Waal (vdW) distance from both the −1 and +1 subsites. Trp87 has no corresponding residue in any of the GH3 enzymes with known structure. In both the xylose-bound and the 4-thioxylobiose-bound Xyl3A structure models, the sidechain of Trp87 has vdW interactions with the C5 atom of the xylose residue bound in subsite −1 and fills the space where a C6 atom and O6 hydroxyl group would be located if the xylose was substituted with glucose.

Also the sulfur atom of Cys292, which forms a cysteine bridge with Cys324, is within vdW distance of the ligand C5 atom in −1. While the sidechain of Cys292 points in another direction, the backbone atoms superpose well with those of Trp286 in HvExo1. This tryptophan was suggested to form one of the edges in a “molecular clamp” around the +1 subsite of the HvExo1 enzyme. Xyl3A is lacking such kind of clamp structure, instead the +1 subsite is surrounded by residues on three sides.

Glu89 in Xyl3A corresponds to the key residue Asp58 in TnBgl3B that has shown to be conserved in 200 GH3 members and involved in keeping the stereochemistry correct for the glucose residue bound in subsite −1. See, Pozzo et al. (2010) Structural and functional analyses of beta-glucosidase 3B from Thermotoga neapolitana: a thermostable three-domain representative of glycoside hydrolase 3, J. Mol. Biol. 397 (3): 724-739. The explanation might be that the positioning of Trp87 causes the backbone to move slightly with the consequence that the side chain of an aspartic acid would be too short to fulfill its function. In Xyl3A, Glu89 is forming hydrogen bonds to both the xylose substrate and to Lys206 thereby strengthening the interactions between these three residues.

### Example 6

### Identification of the Structural Determinants of the Substrate Specificity in GH3 Beta-Xylosidase and Beta-Glucosidase

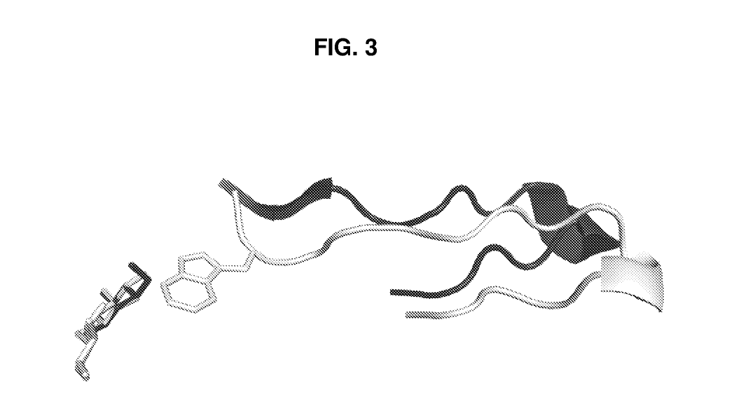

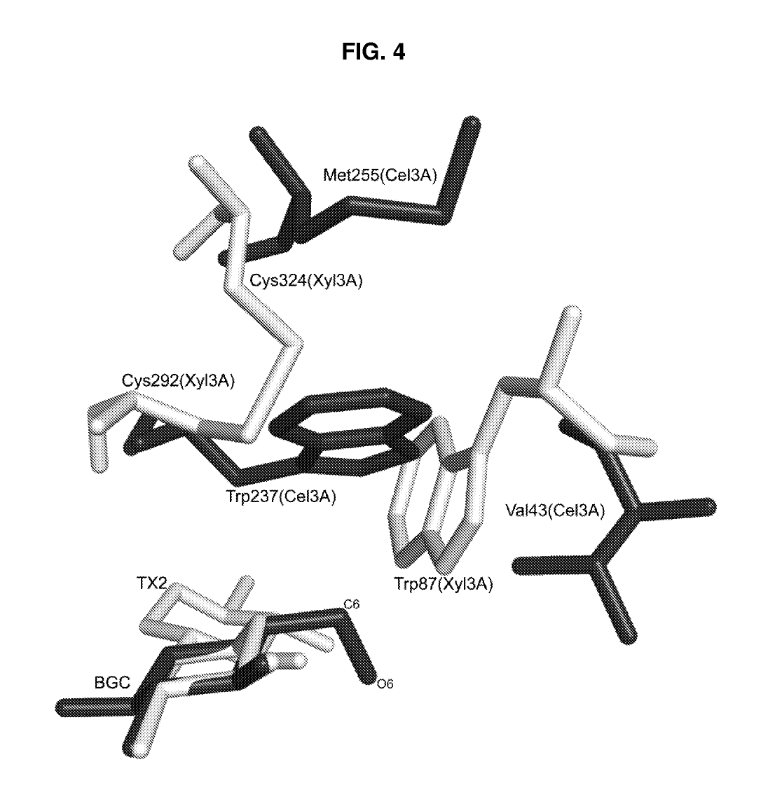

Three amino acid residues have been identified that contribute to the specificity differences between Bgl1 and Xyl3A (FIGS. 3 and 4). For Bgl1 these residues are Val43, Trp237, and Met255. For Xyl3A the corresponding residues are Trp87, Cys292, and Cys324. The latter two Cys residues form a disulfide bridge in the active site in place of Bgl1 Trp237. In Xyl3A another tryptophan, Trp87, takes the place of Cel3A Trp237 but has been rotated such that it occupies the same space as the C6 group of a complexed glucose molecule in Bgl1.

Using the information identified above, it was proposed that, amino acid substitutions that would change the substrate specificity of Bgl1 may include:

Val43: A change of Val43 to a larger hydrophobic side chain would restrict the binding of C6 hydroxyl of glucose. Three changes with increasing side chain length are proposed: L, F, and W.

W237: Each of the Val43 substitutions is extended with changes in W237 to a smaller hydrophobic side chain: L, I, V, A, G.

W237 and M255: Each of the Val43 substitutions is combined with an engineered active site disulfide bridge.

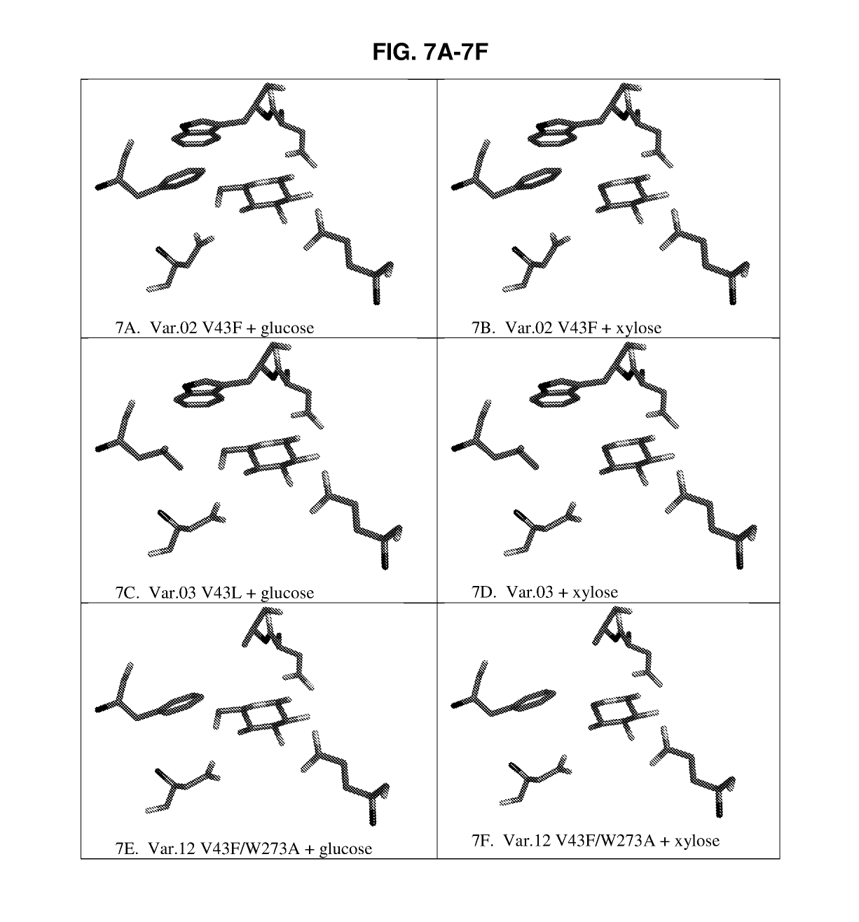

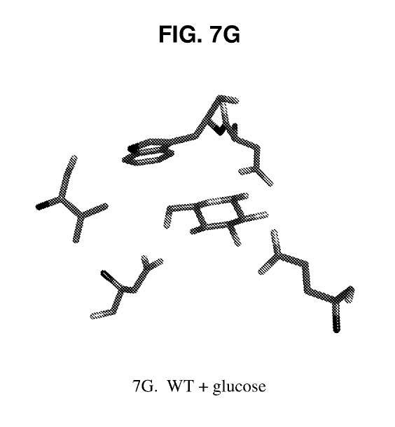

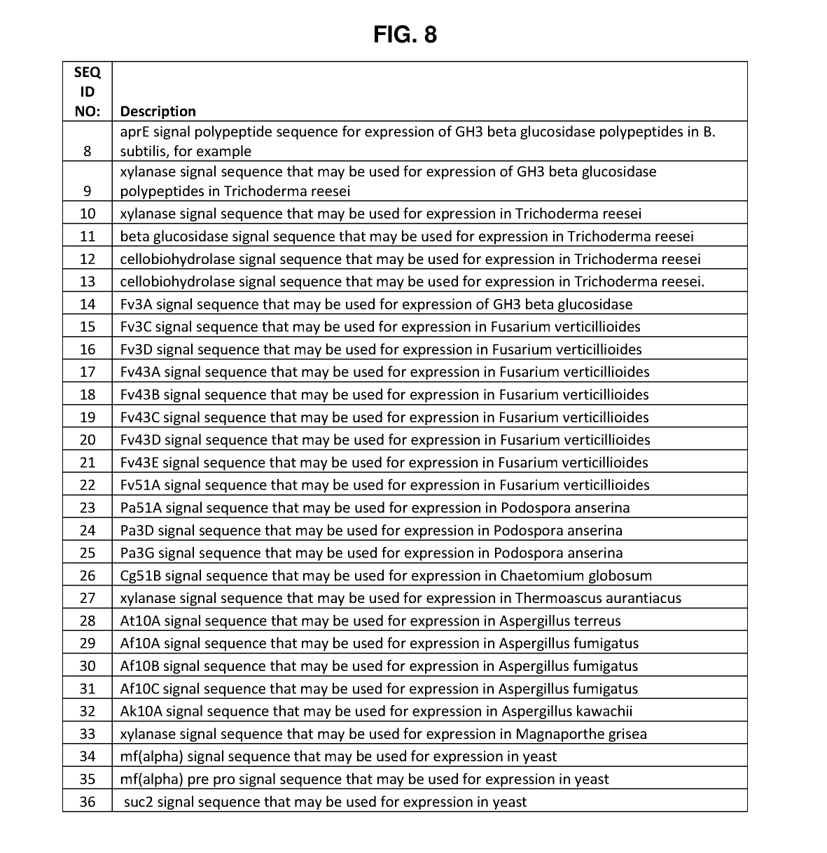

The structural modeling of these variants were presented in FIGS. 7A-7G.

A full list of proposed Bgl1 variants is listed in Table 4.

TABLE 4
Bgl1 variants

| Variant | Sub1 | Sub2 | Sub3 |
|---|---|---|---|
| Bgl1-var-01 | V43W | | |
| Bgl1-var-02 | V43F | | |
| Bgl1-var-03 | V43L | | |
| Bgl1-var-04 | V43W | W237L | |
| Bgl1-var-05 | V43W | W237I | |
| Bgl1-var-06 | V43W | W237V | |
| Bgl1-var-07 | V43W | W237A | |
| Bgl1-var-08 | V43W | W237G | |
| Bgl1-var-09 | V43F | W237L | |
| Bgl1-var-10 | V43F | W237I | |
| Bgl1-var-11 | V43F | W237V | |
| Bgl1-var-12 | V43F | W237A | |
| Bgl1-var-13 | V43F | W237G | |
| Bgl1-var-14 | V43L | W237L | |
| Bgl1-var-15 | V43L | W237I | |
| Bgl1-var-16 | V43L | W237V | |
| Bgl1-var-17 | V43L | W237A | |
| Bgl1-var-18 | V43L | W237G | |
| Bgl1-var-19 | V43W | W237C | M255C |
| Bgl1-var-20 | V43F | W237C | M255C |
| Bgl1-var-21 | V43L | W237C | M255C |

The Bgl1 variants of the table above were produced as follows. The nucleotide sequences encoding these variants were synthesized by an external vendor (BaseClear, Leiden, the Netherlands), and cloned into the pTTTpyr2 vector (see, e.g., published PCT application WO2014029808). Protoplasts of a Trichoderma reesei strain (e.g., the hexa-delete strain of International Publication WO05/001036) with its cbh1, cbh2, eg1, eg2, eg3, and bgl1 deleted) were transformed with plasmid DNA encoding the variants and wild type. The resulting transformants were fermented using standard Trichoderma reesei fermentation procedures.

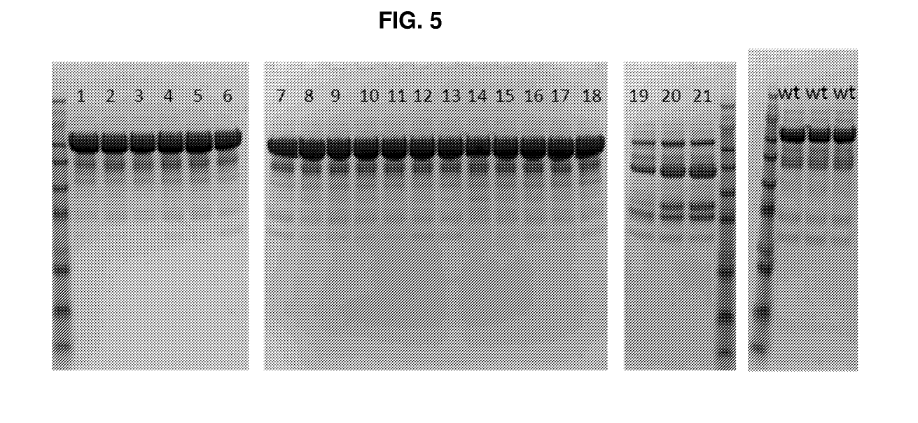

Varied levels of expression were observed with the variants and variants 1-18 appeared to have expressed better than variants 19-21, although even the latter set of variants expressed successfully (FIG. 5).

Initially the variant samples were diluted to 200 to 400 nM and incubated with 1 mM para-nitrophenol-beta-xylopyranoside (pNpX) at 37° C. for 30 minutes. Reactions were stopped by addition of 100 μL of 0.5 M sodium carbonate and absorbance was measured at 410 nm. After background subtraction and normalization to protein concentration, 3 of the variants (Var-02, Var-03, and Var-012) were found to have substantially higher beta-glucosidase activity than that of the wild type T. reesei Bgl1. A performance index (PI) was calculated for each by dividing the background and normalized OD410 values of each of the variants by that of the wild type. The 3 best variants were subject to further studies.

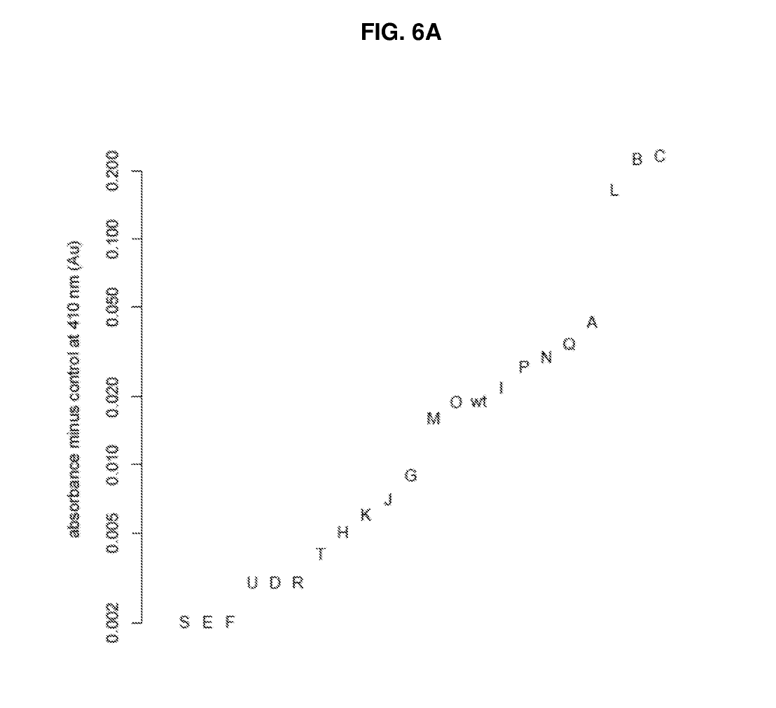

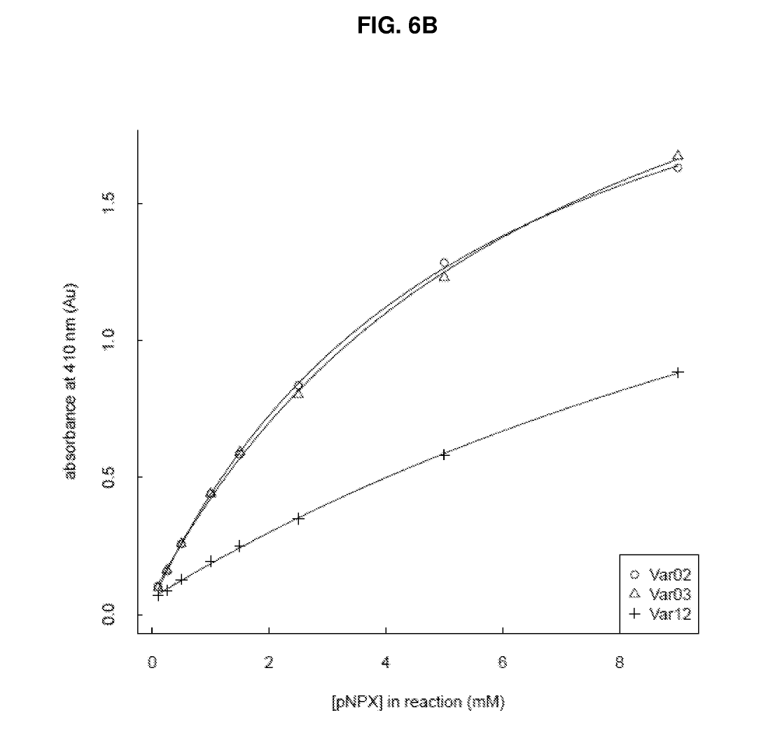

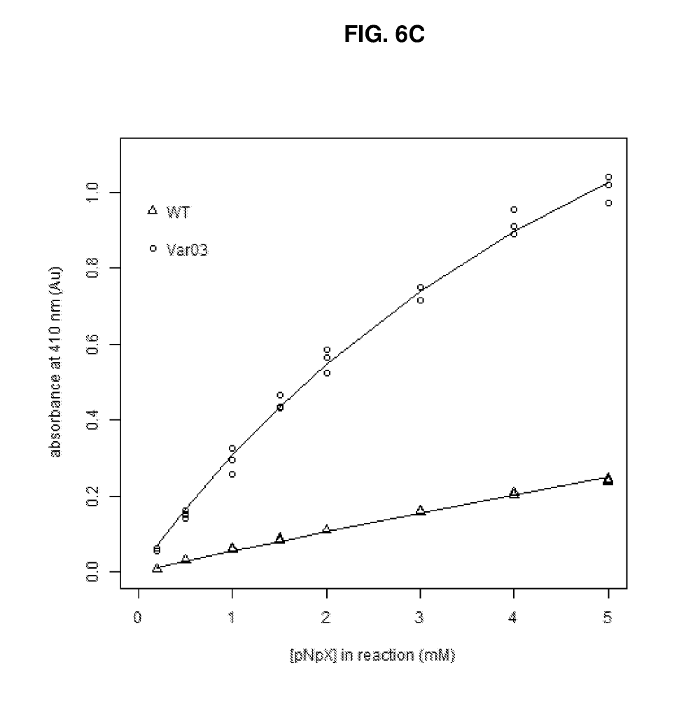

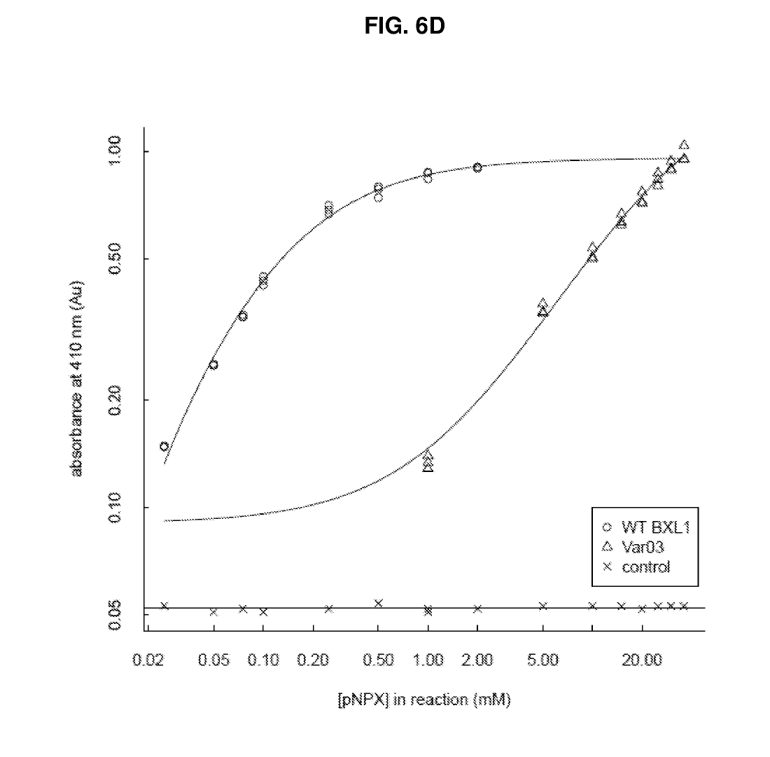

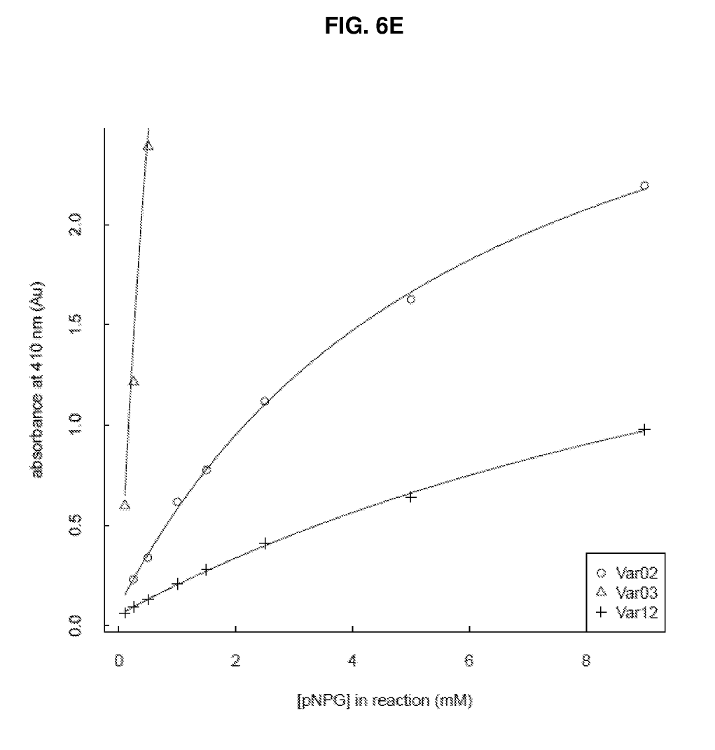

The relative activities of T. reesei Bgl1 and variants for the hydrolysis of 1 mM pNpX have been shown in FIG. 6A and in addition were listed in Table 5:

TABLE 5
Hydrolysis of 1 mM pNpX by Bgl1 WT and variants

| | | Background subtracted and | |
|---|---|---|---|
| Bgl1 var | Substitutions | normalized OD410 | PI |
| Bgl1-var-01 | V43W | 0.043 | 1.13 |
| Bgl1-var-02 | V43F | 0.226 | 5.95 |
| Bgl1-var-03 | V43L | 0.233 | 6.13 |
| Bgl1-var-04 | V43W/W237L | 0.003 | 0.08 |
| Bgl1-var-05 | V43W/W237I | 0.002 | 0.05 |
| Bgl1-var-06 | V43W/W237V | 0.002 | 0.05 |
| Bgl1-var-07 | V43W/W237A | 0.009 | 0.24 |
| Bgl1-var-08 | V43W/W237G | 0.005 | 0.13 |
| Bgl1-var-09 | V43F/W237L | 0.022 | 0.58 |
| Bgl1-var-10 | V43F/W237I | 0.007 | 0.18 |
| Bgl1-var-11 | V43F/W237V | 0.006 | 0.16 |
| Bgl1-var-12 | V43F/W237A | 0.166 | 4.37 |
| Bgl1-var-13 | V43F/W237G | 0.016 | 0.42 |
| Bgl1-var-14 | V43L/W237L | 0.03 | 0.79 |
| Bgl1-var-15 | V43L/W237I | 0.019 | 0.50 |
| Bgl1-var-16 | V43L/W237V | 0.027 | 0.71 |
| Bgl1-var-17 | V43L/W237A | 0.034 | 0.89 |
| Bgl1-var-18 | V43L/W237G | 0.003 | 0.08 |
| Bgl1-var-19 | V43W/W237C/M255C | 0.002 | 0.05 |
| Bgl1-var-20 | V43F/W237C/M255C | 0.004 | 0.11 |
| Bgl1-var-21 | V43L/W237C/M255C | 0.003 | 0.08 |
| Bgl1-WT | | 0.038 | 1.00 |

Variants 02, 03 and 12 were diluted and incubated with varying concentrations of para-nitrophenol-beta-D-xylopyranoside (or para-nitrophenol-beta-D-xyloside) (pNpX) and para-nitrophenol-beta-D-glucopyranoside (pNpG) in the concentration range of 0.1-9 mM at 37° C. for 30 minutes. Reactions were stopped by addition of 100 μL 0.5 M sodium carbonate and absorbance was measured at 410 nm. Background subtracted OD410 absorbances were plotted against substrate concentration (FIG. 6B-E) and the data was fitted with a function for Michaelis-Menten kinetics using the statistical software package R. Michaelis constants and relative maximum velocities for hydrolysis of pNpX and pNpG were reported in Tables 6 and 7, below, respectively.

It was noted that the data for pNpX hydrolysis by the wild type Bgl1 could not be fitted with the Michaelis-Menten function. In order to calculate a Michalis constant the maximum velocity obtained for Variant 03 was used. Variant 02 and Variant 03 displayed 6-7 fold more efficient hydrolysis of pNpX as compared to the wild type Bgl1, while Variant 12 displayed about 2× higher efficiency of that hydrolysis than the wild type (Table 6).

TABLE 6
pNpX hydrolysis by selected Bgl1 variants

| | | | Relative | Rel |
|---|---|---|---|---|
| Variant | Substitutions | Km | Vmax | Vmax/Km |
| Bgl1-var-02 | V43F | 5.7 ± 0.2 | 93 ± 4 | 16.3 |
| Bgl1-var-03 | V43L | 7.0 ± 0.7 | 100 ± 4 | 14.3 |
| Bgl1-var-12 | V43F/W237A | 18.4 ± 1.9 | 89 ± 4 | 4.9 |
| Bgl1-WT | WT | 45.1 ± 0.4 | 100 ± 4* | 2.2 |

*indicates that maximum velocity obtained for Variant 03 was used for calculation of affinity constant of Bgl1-WT

TABLE 7
pNpG hydrolysis

| | | | Relative | Rel |
|---|---|---|---|---|
| Variant | Substitutions | Km | Vmax | Vmax/Km |
| Bgl1-var-02 | V43F | 4.8 ± 0.5 | 46 ± 6 | 9.6 |
| Bgl1-var-03 | V43L | 1.17 ± 0.06 | 87 ± 2 | 74.7 |
| Bgl1-var-12 | V43F/W237A | 9.1 ± 1.9 | 27 ± 10 | 3.0 |
| Bgl1-WT | WT | 1.30 ± 0.06 | 100.0 ± 0.1 | 76.9 |

It was noted that the efficiency of variant 03 for hydrolysis of pNpG was approximately equal to that of Bgl1 wild type whereas the efficiency of variants 02 and 12 were 8× and 26× reduced, respectively (Table 7).

### Comparison of Bgl1 Wild Type and Variant 03 for Hydrolysis of Cellobiose and Xylobiose

Cellobioase and xylobiase activity assays were adapted from Ghose, T. K. “Measurement of Cellulase Activities,” Pure & Appl. Chem., 1987, 59(2): 257-68. Standard error for this assay was determined on a prior occasion to be 10%. The protocol applies the same way for both cellobiose and xylobiose substrates.

Bgl1 wild type and Variant 03 samples were diluted across a microtiter plate (the “dilution plate”) in a sodium acetate buffer 50 mM, at pH 5.0. In a second microtiter plate (the “assay plate”), 50 μL of substrate was added to 50 μL of enzyme solution in each well from the dilution plate. The assay plate was covered and incubated at 50° C. for 30 minutes, shaken at 200 rpm in place in an Innova 44 incubator/shaker. Reaction was quenched with 100 μL 100 mM glycine, in a pH 10 buffer, and gently mixed with pipette. Twenty (20) μL was added from quenched assay plate to 100 μL Millipore water in a HPLC plate. Glucose and cellobiose or xylose and xylobiose concentrations were measured using HPLC.

Using a standard curve, HPLC peak areas were translated to glucose or xylose concentrations in mg/mL. Glucose concentrations were converted to mg produced in the reaction by multiplying the total reaction volume (100 μL=50 μL substrate+50 μL enzyme). Enzyme dilutions were converted to relative concentrations (which were unitless numbers). Enzyme concentrations (mL/mL) were plotted vs. glucose or xylose produced (mg) on a semi-logarithmic scale. The concentration of enzyme required for turnover of 0.1 mg glucose or xylose was determined.

Cellobiase or xylobiase (CB/XB) units were defined as:

CB/XB=(0.185/enzyme concentration to release 0.1 mg glucose or xylose) units mL⁻¹

The specific activities determined for hydrolysis of cellobiose and xylobiose by Bgl1 wild type and Variant 03 were listed in Table 8 below. While Bgl1 Variant 03 has approximately the same cellobiase activity as Bgl1 wild type, its xylobiase activity was improved by over 400×.

TABLE 8
Cellobiase and xylobiase activities of Bgl1 WT and Var.03

| | Cellobiase activity | Xylobiase activity |
|---|---|---|
| Enzyme | (U/mg) | (U/mg) |
| Bgl1 WT | 14.6 | 0.0002 |
| Bgl1 Var.03 | 12.3 | 0.0636 |
| Ratio Var.03/WT | 0.84 | 415 |

Structural Models of Bgl1 Variants with Glucose or Xylose Bound in the Active Site.

The experimentally-determined 3D structure of Bgl1 was used for creating structural models of the active site of Bgl1 variants 2, 3, and 12 (FIG. 7). The amino acid residues at positions 43 and 237 were modified in silico. Glucose was placed in the active site using the coordinates from the Bgl1 structure complexed with glucose. Xylose was transplanted into the active site by structural overlay of Bgl1 with Bxl1 complexed with xylose. V43F complements the space that is occupied by the C6 of glucose and appears to accommodate xylose (FIG. 7B). However, V43F would clash when glucose is present in its original binding mode (FIG. 7A). V43L also complements space that is occupied by the C6 of glucose, but to a lesser extent than V43F (FIG. 7D). Consequently, there appears to be less clashing with glucose bound in its original position (FIG. 7C). W237A creates space in the active site and would result in less interaction with either glucose or xylose (FIGS. 7E and F).

The models may help explain the observed activities of the Bgl1 variants. V43F has increased activity on xylosides, but reduced activity on glucosides. Combination of V43F and W237A reduces the affinity for xylosides, but both affinity and activity for glucosides are reduced. V43L increases the affinity and activity for xylosides, while leaving the hydrolytic activity for glucosides largely unchanged.

### Phylogenetic Analysis of V43L of Bgl1 Variant 03

A number of GH3 beta-glucosidase amino acid sequences were aligned using the alignment program MUSCLE applying the default settings. The alignment was analyzed for the amino acid homologous to T. reesei Bgl1 V43L variant.

Analysis indicated that the majority of these aligned sequences had a valine at the position corresponding to Bgl1 residue 43. This suggested that improved properties observed from the study of T. reesei Bgl1 V43L variant herein could be applied to the other GH3 beta-glucosidases having a sequence identity to SEQ ID NO:2 or 3 at a level as low as 31% (Table 9).

TABLE 9
Amino acid present in Bgl1 homologs
at position homologous to Bgl1 V43

| | | | Seq | |
|---|---|---|---|---|
| SEQ | | | identity | Amino acid |
| ID | | | to | corresponding |
| NO: | Homolog | Organism | Bgl1 | to Bgl1 V43 |
| 37 | TrireBgl1 | Trichoderma reesei | 100% | V |
| 38 | ChaglBglu | Chaetomium globosum | 64% | V |
| 39 | AspteBglu | Aspergillus terreus | 58% | V |
| 40 | SeplyBglu | Septoria lycopersici | 39% | N |
| 41 | PerspBglu | Periconia sp. BCC 2871 | 39% | V |
| 42 | TrireBGL7 | Trichoderma reesei | 38% | A |
| 43 | PenbrBGL | Penicillium brasilianus | 38% | V |
| 44 | PhaavBglu | Phaeosphaeria avenaria | 38% | V |
| 45 | AspfuBGL | Aspergillus fumigatus | 38% | V |
| 46 | AspacBGL1 | Aspergillus aculeatus | 38% | V |
| 47 | TalemBglu | Talaromyces emersonii | 38% | V |
| 48 | TheauBGL | Thermoascus aurentiacus | 38% | V |
| 49 | TrireBGL3 | Trichoderma reesei | 37% | V |
| 50 | AsporBGL1 | Aspergillus oryzae | 37% | V |
| 51 | AspniBGL | Aspergillus niger | 37% | V |
| 52 | KurcaBglu | Kuraishia capsulata | 35% | S |
| 53 | UrofaBglu | Uromyces fabae | 35% | V |
| 54 | SacfiBglu2 | Saccharomycopsis | 34% | V |
| | | fibuligera | | |
| 55 | SacfiBglu1 | Saccharomycopsis | 34% | V |
| | | fibuligera | | |
| 56 | CocimBglu | Coccidioides immitis | 33% | V |
| 57 | PirspBglu | Piromyces sp. E2 | 31% | V |
| 58 | HananBglu | Hansenula anomala | 30% | S |

Although the foregoing compositions and methods has been described in some detail by way of illustration and example for purposes of clarity of understanding, it is readily apparent to those of ordinary skill in the art in light of the teachings herein that certain changes and modifications may be made thereto without departing from the spirit or scope of the appended claims.

Accordingly, the preceding merely illustrates the principles of the present compositions and methods. It will be appreciated that those skilled in the art will be able to devise various arrangements which, although not explicitly described or shown herein, embody the principles of the present compositions and methods and are included within its spirit and scope. Furthermore, all examples and conditional language recited herein are principally intended to aid the reader in understanding the principles of the present compositions and methods and the concepts contributed by the inventors to furthering the art, and are to be construed as being without limitation to such specifically recited examples and conditions. Moreover, all statements herein reciting principles, aspects, and embodiments of the present compositions and methods as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present compositions and methods, therefore, is not intended to be limited to the exemplary embodiments shown and described herein.

1. An engineered beta-glucosidase of glycosyl hydrolyase family 3, comprising an amino acid sequence that is at least 35% identical to that of SEQ ID NO:2, further with at least one substitution at residues 43, 237, or 255, which residues are numbered in reference to the amino acid sequence of SEQ ID NO:3.

2. The engineered beta-glucosidase of claim 1, wherein the substitution is at residue 43 and is the substitution of a valine (V) with a tryptophan (W), a phenylalanine (F) or a leucine (L);
wherein the substitution is at residue 237 and is the substitution of a tryptophan (F) with a leucine (L), an isoleucine (I), a valine (V), an alanine (A), a glycine (G), or a cysteine (C);
or wherein the substitution is at residue 255 and is the substitution of a methionine (M) with a cysteine (C).

3. The engineered beta-glucosidase of claim 1, comprising two or more substitutions at residues 43, 237 and 255, which residues are numbered in reference to the amino acid sequence of SEQ ID NO:3.

4. The engineered beta-glucosidase of claim 3, wherein the two more substitutions are at residues 43 and 237.

5. The engineered beta-glucosidase of claim 3, wherein the two or more substitutions are at residues 43 and 255.

6. The engineered beta-glucosidase of claim 3, wherein the two or more substitutions are at residues 237 and 255.

7. The engineered beta-glucosidase of claim 3, comprising substitutions at all three residues 43, 237 and 255.

8. The engineered beta-glucosidase of claim 1, wherein the engineered beta-glucosidase has at least 2% of beta-xylosidase activity of purified Trichoderma reesei beta xylosidase 3 (Xyl3A) as measured using a standard assay measuring the hydrolysis of substrate para-nitrophenol-beta-D-xyloside (pNpX), or has at least 2% higher beta-xylosidase activity than that of its native, unengineered parent beta-glucosidase.

9. The engineered beta-glucosidase of claim 1, wherein the engineered beta-glucosidase retains at least 30% of its parent, unengineered beta-glucosidase activity, as measured using a standard assay measuring the hydrolysis of para-nitrophenol-beta-D-glucopyranoside (pNpG).

10. A polynucleotide encoding an engineered beta-glucosidase of glycosyl hydrolase family 3, having a polynucleotide sequence that is at least 35% identity to SEQ ID NO:1, and encodes one or more substitution amino acid residues at amino acid residues 43, 237 or 255, which amino acid residues are numbered with reference to SEQ ID NO:3.

11. The polynucleotide of claim 10, further comprising a polynucleotide sequence encoding a native or non-native signal peptide, which signal peptide comprises an amino acid sequence that is at least 90% identity to any one of SEQ ID NO:8-36.

12. An expression vector comprising a polynucleotide encoding the polypeptide of claim 1.

13. A host cell expressing the expression vector of claim 12.

14. The host cell of claim 13, which is a bacterial or a fungal cell.

15. A method of producing an engineered GH3 beta-glucosidase polypeptide comprising an amino acid sequence that is at least 35% identical to SEQ ID NO:2 and with one or more substitution at amino acid residues 43, 237, or 255, which amino acid residues are numbered with reference to SEQ ID NO:3, comprising culturing the host cell of claim 13, under suitable conditions to produce the polypeptide.

16. A composition comprising a culture medium produced by the method of claim 15.

17. A composition comprising the engineered GH3 beta-glucosidase polypeptide of claim 1, further comprising at least one cellulase.

18. A composition comprising the engineered GH3 beta-glucosidase polypeptide of claim 1, further comprising at least one hemicellulase.

19. A method of hydrolyzing a lignocellulosic biomass substrate, comprising contacting the substrate with the polypeptide of claim 1.

20. The method of claim 19, wherein the lignocellulosic biomass substrate is one that has been subjected to a pretreatment.


SEQUENCE LISTING (58 sequences)

SEQ ID NO: 1 - DNA, 2238 bp, artificial sequence
  synthetic construct
atgcgctaccgcaccgctgccgctttagccttagccaccggccccttcgccagagccgatagccacagcacctccggcgctagtgctgaagctgttgtccctcctgctggcaccccttggggcaccgcctacgacaaggccaaggccgccctcgccaagctcaacctccaggacaaggtcggcatcgtcagcggcgtcggctggaacggcggtccctgcgtcggcaacaccagccccgccagcaagatcagctaccccagcctctgcctccaggacggccccctcggcgtccgctacagcaccggcagcaccgccttcacccctggcgtccaggccgccagcacctgggacgtcaacctcatccgcgagcgcggccagttcatcggcgaagaggtcaaggccagcggcatccacgtcatcctcggtcccgttgctggtcccttaggcaagaccccccagggcggtcgcaactgggagggcttcggcgtcgacccctacctcaccggcattgccatgggccagaccatcaacggcatccagagcgtcggcgtccaggccaccgccaagcactacatcctcaacgagcaagagttaaaccgcgagactatcagcagcaaccccgacgaccgcaccctccacgagttatacacctggcccttcgccgacgccgtccaggccaacgtcgccagcgtcatgtgcagctacaacaaggtcaacaccacctgggcctgcgaggaccagtacaccctccagaccgtcctcaaggaccagctcggcttccccggctacgtcatgaccgactggaacgcccagcacaccaccgtccagagcgccaacagcggcctcgacatgagcatgcccggcaccgacttcaacggcaacaaccgcctctggggccctgccctcaccaacgccgtcaacagcaaccaggtccccacctcccgcgtcgacgacatggtcacccgcatcctcgccgcctggtacttaaccggccaagaccaggctggctatcccagcttcaacatcagccgcaacgtccagggcaaccacaagaccaacgtccgcgccattgcccgcgacggcatcgtcctcctcaagaacgacgccaacatcctccccctcaagaagcccgcctctatcgccgtcgtcggcagcgccgccatcatcggcaaccacgcccgcaacagccccagctgcaacgacaagggctgcgatgacggtgccctcggcatgggctggggctctggcgccgtcaactacccctacttcgtcgccccctacgacgccatcaacacccgcgccagcagccagggcacccaggtcaccctcagcaacaccgacaatacttcttctggcgcttctgctgctagaggcaaggacgtcgccatcgtttttatcactgccgattctggcgaaggctacatcaccgtcgagggcaacgccggcgaccgcaacaacctcgacccctggcacaacggcaatgccctcgtccaggccgttgctggtgctaacagcaacgtcatcgtcgtcgtccacagcgtcggcgccatcatcctcgagcagatcctcgccctcccccaggtcaaggccgtcgtctgggccggcttacccagccaggaaagcggcaacgccttagtcgacgtcctctggggtgacgtttccccctctggcaagctcgtctacaccattgccaagagccccaacgactacaacacccgcattgtcagcggcggcagcgacagcttcagcgagggcctcttcatcgactacaagcacttcgacgacgccaacattaccccccgctacgagttcggctacggcctcagctacaccaagttcaactacagccgcctcagcgtcctcagcaccgccaagagcggccctgccactggtgctgtcgtccctggtggcccttctgacctcttccagaacgtcgccacggtcaccgtcgacattgccaactccggccaggtcactggcgccgaggtcgcccagctctacatcacctaccccagcagcgcccctcgcactcctcccaagcagctcagaggcttcgctaagttaaacttaacccctggccagagcggcaccgccacctttaacatccgcagacgcgacctcagctactgggacaccgccagccagaagtgggtcgtccccagcggcagcttcggcatctccgtcggcgccagctcccgcgacatccgcctcaccagcaccctcagcgtcgcctgatga

SEQ ID NO: 2 - protein, 744 aa, Trichoderma reesei
MRYRTAAALALATGPFARADSHSTSGASAEAVVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 3 - protein, 713 aa, Trichoderma reesei
VVPPAGTPWGTAYDKAKAALAKLNLQDKVGIVSGVGWNGGPCVGNTSPASKISYPSLCLQDGPLGVRYSTGSTAFTPGVQAASTWDVNLIRERGQFIGEEVKASGIHVILGPVAGPLGKTPQGGRNWEGFGVDPYLTGIAMGQTINGIQSVGVQATAKHYILNEQELNRETISSNPDDRTLHELYTWPFADAVQANVASVMCSYNKVNTTWACEDQYTLQTVLKDQLGFPGYVMTDWNAQHTTVQSANSGLDMSMPGTDFNGNNRLWGPALTNAVNSNQVPTSRVDDMVTRILAAWYLTGQDQAGYPSFNISRNVQGNHKTNVRAIARDGIVLLKNDANILPLKKPASIAVVGSAAIIGNHARNSPSCNDKGCDDGALGMGWGSGAVNYPYFVAPYDAINTRASSQGTQVTLSNTDNTSSGASAARGKDVAIVFITADSGEGYITVEGNAGDRNNLDPWHNGNALVQAVAGANSNVIVVVHSVGAIILEQILALPQVKAVVWAGLPSQESGNALVDVLWGDVSPSGKLVYTIAKSPNDYNTRIVSGGSDSFSEGLFIDYKHFDDANITPRYEFGYGLSYTKFNYSRLSVLSTAKSGPATGAVVPGGPSDLFQNVATVTVDIANSGQVTGAEVAQLYITYPSSAPRTPPKQLRGFAKLNLTPGQSGTATFNIRRRDLSYWDTASQKWVVPSGSFGISVGASSRDIRLTSTLSVA

SEQ ID NO: 4 - protein, 797 aa, Trichoderma reesei
MVNNAALLAALSALLPTALAQNNQTYANYSAQGQPDLYPETLATLTLSFPDCEHGPLKNNLVCDSSAGYVERAQALISLFTLEELILNTQNSGPGVPRLGLPNYQVWNEALHGLDRANFATKGGQFEWATSFPMPILTTAALNRTLIHQIADIISTQARAFSNSGRYGLDVYAPNVNGFRSPLWGRGQETPGEDAFFLSSAYTYEYITGIQGGVDPEHLKVAATVKHFAGYDLENWNNQSRLGFDAIITQQDLSEYYTPQFLAAARYAKSRSLMCAYNSVNGVPSCANSFFLQTLLRESWGFPEWGYVSSDCDAVYNVFNPHDYASNQSSAAASSLRAGTDIDCGQTYPWHLNESFVAGEVSRGEIERSVTRLYANLVRLGYFDKKNQYRSLGWKDVVKTDAWNISYEAAVEGIVLLKNDGTLPLSKKVRSIALIGPWANATTQMQGNYYGPAPYLISPLEAAKKAGYHVNFELGTEIAGNSTTGFAKAIAAAKKSDAIIYLGGIDNTIEQEGADRTDIAWPGNQLDLIKQLSEVGKPLVVLQMGGGQVDSSSLKSNKKVNSLVWGGYPGQSGGVALFDILSGKRAPAGRLVTTQYPAEYVHQFPQNDMNLRPDGKSNPGQTYIWYTGKPVYEFGSGLFYTTFKETLASHPKSLKFNTSSILSAPHPGYTYSEQIPVFTFEANIKNSGKTESPYTAMLFVRTSNAGPAPYPNKWLVGFDRLADIKPGHSSKLSIPIPVSALARVDSHGNRIVYPGKYELALNTDESVKLEFELVGEEVTIENWPLEEQQIKDATPDA

SEQ ID NO: 5 - protein, 777 aa, Trichoderma reesei
QNNQTYANYSAQGQPDLYPETLATLTLSFPDCEHGPLKNNLVCDSSAGYVERAQALISLFTLEELILNTQNSGPGVPRLGLPNYQVWNEALHGLDRANFATKGGQFEWATSFPMPILTTAALNRTLIHQIADIISTQARAFSNSGRYGLDVYAPNVNGFRSPLWGRGQETPGEDAFFLSSAYTYEYITGIQGGVDPEHLKVAATVKHFAGYDLENWNNQSRLGFDAIITQQDLSEYYTPQFLAAARYAKSRSLMCAYNSVNGVPSCANSFFLQTLLRESWGFPEWGYVSSDCDAVYNVFNPHDYASNQSSAAASSLRAGTDIDCGQTYPWHLNESFVAGEVSRGEIERSVTRLYANLVRLGYFDKKNQYRSLGWKDVVKTDAWNISYEAAVEGIVLLKNDGTLPLSKKVRSIALIGPWANATTQMQGNYYGPAPYLISPLEAAKKAGYHVNFELGTEIAGNSTTGFAKAIAAAKKSDAIIYLGGIDNTIEQEGADRTDIAWPGNQLDLIKQLSEVGKPLVVLQMGGGQVDSSSLKSNKKVNSLVWGGYPGQSGGVALFDILSGKRAPAGRLVTTQYPAEYVHQFPQNDMNLRPDGKSNPGQTYIWYTGKPVYEFGSGLFYTTFKETLASHPKSLKFNTSSILSAPHPGYTYSEQIPVFTFEANIKNSGKTESPYTAMLFVRTSNAGPAPYPNKWLVGFDRLADIKPGHSSKLSIPIPVSALARVDSHGNRIVYPGKYELALNTDESVKLEFELVGEEVTIENWPLEEQQIKDATPDA

SEQ ID NO: 6 - DNA, 23 bp, artificial sequence
  primer
caccatggtgaataacgcagctc

SEQ ID NO: 7 - DNA, 21 bp, artificial sequence
  primer
ttatgcgtcaggtgtagcatc

SEQ ID NO: 8 - protein, 29 aa, Bacillus subtilis
MRSKKLWISLLFALTLIFTMAFSNMSAQA

SEQ ID NO: 9 - protein, 32 aa, Trichoderma reesei
MVSFTSLLAASPPSRASCRPAAEVESVAVEKR

SEQ ID NO: 10 - protein, 16 aa, Trichoderma reesei
MKANVILCLLAPLVAA

SEQ ID NO: 11 - protein, 19 aa, Trichoderma reesei
MRYRTAAALALATGPFARA

SEQ ID NO: 12 - protein, 18 aa, Trichoderma reesei
MIVGILTTLATLATLAAS

SEQ ID NO: 13 - protein, 17 aa, Trichoderma reesei
MYRKLAVISAFLATARA

SEQ ID NO: 14 - protein, 23 aa, Fusarium verticillioides
MLLNLQVAASALSLSLLGGLAEA

SEQ ID NO: 15 - protein, 19 aa, Fusarium verticillioides
MKLNWVAAALSIGAAGTDS

SEQ ID NO: 16 - protein, 19 aa, Fusarium verticillioides
MASIRSVLVSGLLAAGVNA

SEQ ID NO: 17 - protein, 22 aa, Fusarium verticillioides
MWLTSPLLFASTLLGLTGVALA

SEQ ID NO: 18 - protein, 16 aa, Fusarium verticillioides
MRFSWLLCPLLAMGSA

SEQ ID NO: 19 - protein, 22 aa, Fusarium verticillioides
MRLLSFPSHLLVAFLTLKEASS

SEQ ID NO: 20 - protein, 20 aa, Fusarium verticillioides
MQLKFLSSALLLSLTGNCAA

SEQ ID NO: 21 - protein, 18 aa, Fusarium verticillioides
MKVYWLVAWATSLTPALA

SEQ ID NO: 22 - protein, 19 aa, Fusarium verticillioides
MVRFSSILAAAACFVAVES

SEQ ID NO: 23 - protein, 20 aa, Podospora anserine
MIHLKPALAALLALSTQCVA

SEQ ID NO: 24 - protein, 17 aa, Podospora anserine
MALQTFFLLAAAMLANA

SEQ ID NO: 25 - protein, 19 aa, Podospora anserine
MKLNKPFLAIYLAFNLAEA

SEQ ID NO: 26 - protein, 20 aa, Chaetomium globosum
MAPLSLRALSLLALTGAAAA

SEQ ID NO: 27 - protein, 19 aa, Thermoascus aurantiacus
MVRPTILLTSLLLAPFAAA

SEQ ID NO: 28 - protein, 21 aa, Aspergillus terreus
MHMHSLVAALAAGTLPLLASA

SEQ ID NO: 29 - protein, 19 aa, Aspergillus fumigatus
MVHLSSLAAALAALPLVYG

SEQ ID NO: 30 - protein, 17 aa, Aspergillus fumigatus
MRFSLAATTLLAGLATA

SEQ ID NO: 31 - protein, 19 aa, Aspergillus fumigatus
MVVLSKLVSSILFASLVSA

SEQ ID NO: 32 - protein, 19 aa, Aspergillus kawachii
MVQIKAAALAMLFASHVLS

SEQ ID NO: 33 - protein, 17 aa, Magnaporthe grisea
MKASSVLLGLAPLAALA

SEQ ID NO: 34 - protein, 19 aa, Saccharomyces cerevisiae
MRFPSIFTAVLFAASSALA

SEQ ID NO: 35 - protein, 85 aa, Saccharomyces cerevisiae
MRFPSIFTAVLFAASSALAAPVNTTTEDETAQIPAEAVIGYLDLEGDFDVAVLPFSNSTNNGLLFINTTIASIAAKEEGVSLDKR

SEQ ID NO: 36 - protein, 20 aa, Saccharomyces cerevisiae
MLLQAFLFLLAGFAAKISAR

SEQ ID NO: 37 - protein, 744 aa, Trichoderma reesei; identical to SEQ ID NO: 2

SEQ ID NO: 38 - protein, 726 aa, Chaetomium globosum
MTTLRNFALLAAAVLARVEALEAADWAAAEASAKTALAKMSQQDKISIVTGIGWDKGPCVGNTAAINSINYPQLCLQDGPLGIRFGTGSTAFTPGVQAASTWDTELMRQRGEYLGAEAKGCGIHVLLGPVAGALGKIPHGGRNWEGFGTDPYLAGIAMAETIEGLQSAGVQACAKHYIVNEQELNRETISSDVDDRTMHELYLWPFADAVHANVASVMCSYNKINGSWGCENDHAQNGLLKKELGFKGYVVSDWNAQHTTDGAANNGMDMTMPGSDYNGNNVLWGPQLSNAVNSNRVSRDRLDDMAKRILTSWYLLGQNSGYPNININANVQGNHKENVRAVARDGIVLLKNDEGVLPLKKPGKVALVGSAASVNSAGPNACVDKGCNTGALGMGWGSGSVNYPYFVAPYDALKTRAQADGTTLSLHNSDSTNGVSGVVSGADVAIVVITADSGEGYITVEGHAGDRNHLDPWHDGNALVKAVAAANKNTIVVVHSTGPIILETILATEGVKAVVWAGLPSQENGNALVDVLYGLTSPSGKLVYSIAKRPEDYGTAPSKGSNDKFTEGLFVDYRHFDNAKIEPRYEFGFGLSYTEFTYADLSVTSTVTAGPASGETIPGGAADLWETVATVTASITNSGEVEGAEVAQLYITLPSAAPSTPPKQLRGFAKLKLEPGASGVATFNLRRRDLSYWDAGRGQWVVPAGEFTVSVGASSRDVRLTGSLTA

SEQ ID NO: 39 - protein, 736 aa, Aspergillus terreus
MNYRVPSLKATALAMAALTQALTTWDAAYEKALADLASLTQSEKVGVVSGITWEGGPCVGNTYAPESIAYPSLCLQDGPLGIRFANPVTAFPAGINAGATWDRELLRARGAAMGEEAKGLGVHVQLAPVAGALGKIPSAGRNWEGFTSDPYLSGIAMAETIHGMQGSGVQACAKHYILNEQEHSRETISSNVDDRTMHEVYLWPFYDAVKANVASVMCSYNKINGTWACENEGILDTLLKQELGFRGYVMSDWNAQHSTVASANTGLDMTMPGSDFSQPPGSIYWNENLAEAVANGSVPQARVDDMVTRILAAWYLLEQDQGYPAVAFDSRNGGKASVDVTADHADIARTVARDSIVLLKNSNNTLPLRNPSSIAVVGSDAIVNPDGPNACTDRGCNVGTLAQGWGSGTAEFPYLVAPLDAIQERSSGNGTKVVTSTTDDATAGADAAASADIAIVFISSDSGEGYITVEGHQGDRNNLDPWHGGNDLVKAVAAVNKKTIVVVHSTGPVVLETILAQPNVVAVVWAGIPGQESGNALADVLYGDVSPSGKLPYTIGKSEADYGTTWVANGADDDFPEGLFIDYRHFDKNEIEPRYEFGFGLSYTRFNFSNLAINIDATSGPTSGAVDVGGAADLYDSVGTISATVTNVGGVSGAEVAQLYIGFPSSAPETPPKQLRGFQKLPLAGGADGVAEFELTRRDISYWDVGQQKWVVPEGSFQVYVGASSRDIRLDGSFTV

SEQ ID NO: 40 - protein, 803 aa, Septoria lycopersici
MVSSLFNIAALAGAVIALSHEDQSKHFTTIPTFPTPDSTGEGWKAAFEKAADAVSRLNLTQKVALTTGTTAGLSCNGNIAPIPEINFSGLCLADGPVSVRIADLATVFPAGLTAAATWDRQLIYERARALGSEFRGKGSQVHLGPASGALGRHPLGGRNWESFSPDPYLSGVAMDFSIRGIQEMGVQANRKHFIGNEQETQRSNTFTDDGTEIQAISSNIDDRTMHELYLWPFANAVRSGVASVMCSYNRLNQTYACENSKLMNGILKGELGFQGYVVSDWYATHSGVESVNAGLDMTMPGPLDSPSTALRPPPSYLGGNLTEAVLNGTIPEARVDDMARRILMPYFFLGQDTDFPTVDPSTGFVFARTYNYPDEYLTLGGLDPYNPPPARDVRGNHSDIVRKVAAAGTVLLKNVNNVLPLKEPKSVGIFGNGAADVTEGLTFTGDDSGPWGADIGALSVGGGSGAGRHTHLVSPLAAIRKRTESVGGRVQYLLSNSRIVNDDFTSIYPTPEVCLVFLKTWAREGTDRLSYENDWNSTAVVNNVARRCPNTIVVTHSGGINTMPWADNANVTAILAAHYPGQENGNSIMDILYGDVNPSGRLPYTIPKLATDYDFPVVNITNEAQDPYVWQADFTEGLLIDYRHFDARNITPLYEFGYGLSYTTFEIEGVANLVAKSAKLSAFPASTDISHPGGNPDLWEEVVSVTAAVKNTGSVSGSQVVQLYISLPADGIPENSPMQVLRGFEKVDLQPGQSKSVEFSIMRRDLSFWNTTAQDWEIPNGQIEFRVGFSSRDIKSIVSRSFL

SEQ ID NO: 41 - protein, 866 aa, unknown
  Periconia sp. BCC 2871
MASWLAPALLAVGLASAQAPFPNGSSPLNDITSPPFYPSPWMDPSAAGWAEAYTKAQAFVRQLTLLEKVNLTTGVGWEGEACVGNTGSIPRLGFPGFCTQDSPLGVRFADYVSAFTAGGTIAASWDRSEFYRRGYQMGVEHRGKGVDVQLGPVVGPIGRHPKGGRNWEGFSPDPVLSGIAVAETVKGIQDAGVIACTKHFILNEQEHFRQPGNVGDFGFVDAVSANLADKTLHELYLWPFADAVRAGTGSIMCSYNKANNSQVCQNSYLQNYILKGELGFQGFTMSDWDAQHSGVASTLAGLDMNMPGDTDFDSGFSFWGPNMTLSIINGTVPEWRLDDAATRIMAAYYLVGRDRHAVPVNFNSWSKDTYGYQHAYAKVGYGLINQHVDVRADHFKSIRTAAAKSTVLLKNNGVLPLKGTEKYTAVFGNDAGEAQYGPNGCADHGCDNGTLAMGWGSGTADYPYLVTPLEAIKRTVGDHGGVIASVTDNYAFSQIMALAKQATHAIVFVNADSGEGYITVDGNEGDRNNLTLWQNGEELVRNVSGYCNNTIVVIHSVGPVLVDSFNNSPNVSAILWAGLPGQESGNAITDVLYGRVNPGGKLPFTIGKSAEEYGPDIIYEPTAGHGSPQANFEEGVFIDYRSFDKKNITPVYEFGFGLSYTNFSYSNLVVTRVNAPAYVPTTGNTTAAPTLGNSSKDASDYQWPANLTYVNKYIYPYLNSTDLKEASNDPEYGIEHEYPEGATDGSPQPRIAAGGGPGGNPQLWDVLYKVTATVTNNGAVAGDEVAQLYVSLGGPEDPPVVLRNFDRLTIAPGQSVEFTADITRRDVSNWDTVSQNWVISNSTKTVYVGASSRKLPLKATLPSSSY

SEQ ID NO: 42 - protein, 765 aa, Trichoderma reesei
MRLCDLSSLASWVLVTVALPSSGAAAKGVSQIPSTHSSQSKGNGPWAHAYRRAEKLVRQMTLEEKANITRGFTGDNVCAGNTGSVPRLGWPGMCVHDAGNGVRATDLVNSYPSGIHVGASWDRNLTYERGLHMGGEFKAKGVNVPLGPNAGPLGRTPLGGRNWEGFSIDPYLSGQLNAETITGMQDAGVIANIKHFIANEQETLRRPYFGVEAVSANIDDRTLHEYYLWPFMDSVHAGVGSVMCSYNRINNTYGCMNDKLMNGILKAELGFQGFVMLDWNAQHDLQSANAGLDMVMPLGGSWGKNLTDAVANGTVSESRITDMATRIIAAWYLVGQDGNNFPVPGIGLKQLTKPHEQVDARDPASKPVLLEGAIAGHVLVKNENNALPFNKKLTMISVFGYDATIPRTKNTDILFQLGYTSSPEMAQAVLGNEAHFDQAAKGGTIMTGGRAGANAPSYIDDPLAAIQRRARKDDTWVNWDLDSFNPEVNAASDACLVFINAIATEGWDRDGLHDDFSDGLVLNVAANCSNTIVVVHAAGTRLVDQWIEHPNVTAAVIAHLPGQDSGRALVKLLYGEANFSGKLPYTIAKNESDYSVYTPCQRRSPEDTDPQCDFTEGVYLDYRAFDANNMTPRFEFGYGLSYTSFNYSALSIKKAKGLRQSRCTDDLWQAAAQVTASITNSGGMSGSEVAQLYLAIPNSPPKQLRGFNKLLLRPHESGTVHFGLTKRDLSVWDVVSQSWVIQEGEYKVFVGASSRDIRLSGKLHI

SEQ ID NO: 43 - protein, 878 aa, Penicillium brasilianus
MQGSTIFLAFASWASQVAAIAQPIQKHEPGFLHGPQAIESFSEPFYPSPWMNPHAEGWEAAYQKAQDFVSQLTILEKINLTTGVGWENGPCVGNTGSIPRLGFKGFCTQDSPQGVRFADYSSAFTSSQMAAATFDRSILYQRGQAMAQEHKAKGITIQLGPVAGPLGRIPEGGRNWEGFSPDPVLTGIAMAETIKGMQDTGVIACAKHYIGNEQEHFRQVGEAAGHGYTISDTISSNIDDRAMHELYLWPFADAVRAGVGSFMCSYSQINNSYGCQNSQTLNKLLKSELGFQGFVMSDWGAHHSGVSSALAGLDMSMPGDTEFDSGLSFWGSNLTIAILNGTVPEWRLDDMAMRIMAAYFKVGLTIEDQPDVNFNAWTHDTYGYKYAYSKEDYEQVNWHVDVRSDHNKLIRETAAKGTVLLKNNFHALPLKQPRFVAVVGQDAGPNPKGPNGCADRGCDQGTLAMGWGSGSTEFPYLVTPDTAIQSKVLEYGGRYESIFDNYDDNAILSLVSQPDATCIVFANADSGEGYITVDNNWGDRNNLTLWQNADQVISTVSSRCNNTIVVLHSVGPVLLNGIYEHPNITAIVWAGMPGEESGNALVDILWGNVNPAGRTPFTWAKSREDYGTDIMYEPNNGQRAPQQDFTESIYLDYRHFDKAGIEPIYEFGFGLSYTTFEYSDLRVVKKYVQPYSPTTGTGAQAPSIGQPPSQNLDTYKFPATYKYIKTFIYPYLNSTVSLRAASKDPEYGRTDFIPPHARDGSPQPLNPAGDPVASGGNNMLYDELYEVTAQIKNTGDVAGDEVVQLYVDLGGDNPPRQLRNFDRFYLLPGQSSTFRATLTRRDLSNWDIEAQNWRVTESPKRVYVGRSSRDLPLSSQLE

SEQ ID NO: 44 - protein, 871 aa, Phaeosphaeria avenaria
MALAVAFFVTQVLAQQYPTSNTSSPAANSSSPLDNAVSPPFYPSPWIEGLGDWEAAYQKAQAFVSQLTLLEKVNLTTGTGWQSDHCVGNTGGVPRLNFTGICNQDAPLGVRFADYVSAFPSGGTIAAAWDRGEWYLRGYQMGSEHRSKGVDVQLGPVVGPLGRNPKGGRNWEGFSPDPYLSGIASAESVRGIQDAGVIACTKHYIMNEQEHFRQPGNFEDQGFVDALSSNLDDKTLHELYLWPFADAVRAGTGSIMCSYNKVNNSQACQNSYLQNYILKGELGFQGFIMSDWDAQHSGVASTFAGLDMTMPGDTDFNSGKTFWGTNFTTSILNGTVPQWRLDDAVTRIMAAFYYVGRDKARIPVNFDSWSRDTYGFDHYYGKAGYSQINSHVDVRADHFRSIRRTAAMSTVLLKNEGALPLTGSEKWTAVFGDDAGEGQLGPNGFPDHGGNNGTLAMGWGSGTSDYPYLVTPLESIKATVAQNGGIVTSVTDNWAYTQIQTLAKQASVAIVFVNADSGEGYITVDGNAGDRNNLTLWQDGDTLIKNVSSLCNNTIVVIHSVGPVLVNSFYDSENVTAILWAGLPGQESGNAIADILYGRHNPGGKLPFTIGSDAAEYGPDLIYEPTNNSSSPQDNFEEGVFIDYRAFDKQNVTPIYEFGFGLSYTKFSYSNLTVKKANAGAYTPATGQSKAAPTLGNFSTDASQYQWPSDFTYIDTFIYPYLNSTDLKTASQDPEYGLNYTWPAGATDGTPQARIPAGGAPGGNPQLWDVLFSVEATITNNGTVPGDEVVQLYVSLGNPDDPKIVLRGFDRLSIQPGKTATFHADITRRDVSNWDVASQNWVITSAPKTVYVGASSRKLPLTATLDTSDFQ

SEQ ID NO: 45 - protein, 871 aa, Aspergillus fumigatus
MRFGWLEVAALTAASVANAQVFDNSHGNNQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPALTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPY

SEQ ID NO: 46 - protein, 860 aa, Aspergillus aculeatus
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 47 - protein, 857 aa, Talaromyces emersonii
MRNGLLKVAALAAASAVNGENLAYSPPFYPSPWANGQGDWAEAYQKAVQFVSQLTLAEKVNLTTGTGWEQDRCVGQVGSIPRLGFPGLCMQDSPLGVRDTDYNSAFPAGVNVAATWDRNLAYRRGVAMGEEHRGKGVDVQLGPVAGPLGRSPDAGRNWEGFAPDPVLTGNMMASTIQGIQDAGVIACAKHFILYEQEHFRQGAQDGYDISDSISANADDKTMHELYLWPFADAVRAGVGSVMCSYNQVNNSYACSNSYTMNKLLKSELGFQGFVMTDWGGHHSGVGSALAGLDMSMPGDIAFDSGTSFWGTNLTVAVLNGSIPEWRVDDMAVRIMSAYYKVGRDRYSVPINFDSWTLDTYGPEHYAVGQGQTKINEHVDVRGNHAEIIHEIGAASAVLLKNKGGLPLTGTERFVGVFGKDAGSNPWGVNGCSDRGCDNGTLAMGWGSGTANFPYLVTPEQAIQREVLSRNGTFTGITDNGALAEMAAAASQADTCLVFANADSGEGYITVDGNEGDRKNLTLWQGADQVIHNVSANCNNTVVVLHTVGPVLIDDWYDHPNVTAILWAGLPGQESGNSLVDVLYGRVNPGKTPFTWGRARDDYGAPLIVKPNNGKGAPQQDFTEGIFIDYRRFDKYNITPIYEFGFGLSYTTFEFSQLNVQPINAPPYTPASGFTKAAQSFGQPSNASDNLYPSDIERVPLYIYPWLNSTDLKASANDPDYGLPTEKYVPPNATNGDPQPIDPAGGAPGGNPSLYEPVARVTTIITNTGKVTGDEVPQLYVSLGGPDDAPKVLRGFDRITLAPGQQYLWTTTLTRRDISNWDPVTQNWVVTNYTKTIYVGNSSRNLPLQAPLKPYPGI

SEQ ID NO: 48 - protein, 843 aa, Thermoascus aurentiacus
MRLGWLELAVAAAATVASAKDDLAYSPPFYPSPWMNGNGEWAEAYRRAVDFVSQLTLAEKVNLTTGVGWMQEKCVGETGSIPRLGFRGLCLQDSPLGVRFADYVSAFPAGVNVAATWDKNLAYLRGKAMGEEHRGKGVDVQLGPVAGPLGRHPDGGRNWEGFSPDPVLTGVLMAETIKGIQDAGVIACAKHFIGNEMEHFRQAGEAVGYGFDITESVSSNIDDKTLHELYLWPFADAVRAGVGSFMCSYNQVNNSYSCSNSYLLNKLLKSELDFQGFVMSDWGAHHSGVGAALAGLDMSMPGDTAFGTGKSFWGTNLTIAVLNGTVPEWRVDDMAVRIMAAFYKVGRDRYQVPVNFDSWTKDEYGYEHALVGQNYVKVNDKVDVRADHADIIRQIGSASVVLLKNDGGLPLTGYEKFTGVFGEDAGSNRWGADGCSDRGCDNGTLAMGWGSGTADFPYLVTPEQAIQNEILSKGKGLDSVSIVFVNADSGEGYINVDGNEGDRKNLTLWKGGEEVIKTVAANCNNTIVVMHTVGPVLIDEWYDNPNVTAIVWAGLPGQESGNSLVDVLYGRVSPGGKTPFTWGKTRESYGAPLLTKPNNGKGAPQDDFTEGVFIDYRRFDKYNETPIYEFGFGLSYTTFEYSNIYVQPLNARPYTPASGSTKAAPTFGNISTDYADYLYPEDIHKVPLYIYPWLNTTDPEEVLRRSRLTEMKAEDYIPSGATDGSPQPILPAGGAPGGNPGLYDEMYRVSAIITNTGNVVGDEVPQLYVSLGGPDDPKVVLRNFDRITLHPGQQTMWTTTLTRRDISNWDPASQNWVVTKYPKTVYIGSSSRKLHLQAPLPPY

SEQ ID NO: 49 - protein, 874 aa, Trichoderma reesei
MKTLSVFAAALLAAVAEANPYPPPHSNQAYSPPFYPSPWMDPSAPGWEQAYAQAKEFVSGLTLLEKVNLTTGVGWMGEKCVGNVGTVPRLGMRSLCMQDGPLGLRFNTYNSAFSVGLTAAASWSRHLWVDRGTALGSEAKGKGVDVLLGPVAGPLGRNPNGGRNVEGFGSDPYLAGLALADTVTGIQNAGTIACAKHFLLNEQEHFRQVGEANGYGYPITEALSSNVDDKTIHEVYGWPFQDAVKAGVGSFMCSYNQVNNSYACQNSKLINGLLKEEYGFQGFVMSDWQAQHTGVASAVAGLDMTMPGDTAFNTGASYFGSNLTLAVLNGTVPEWRIDDMVMRIMAPFFKVGKTVDSLIDTNFDSWTNGEYGYVQAAVNENWEKVNYGVDVRANHANHIREVGAKGTVIFKNNGILPLKKPKFLTVIGEDAGGNPAGPNGCGDRGCDDGTLAMEWGSGTTNFPYLVTPDAALQSQALQDGTRYESILSNYAISQTQALVSQPDAIAIVFANSDSGEGYINVDGNEGDRKNLTLWKNGDDLIKTVAAVNPKTIVVIHSTGPVILKDYANHPNISAILWAGAPGQESGNSLVDILYGKQSPGRTPFTWGPSLESYGVSVMTTPNNGNGAPQDNFNEGAFIDYRYFDKVAPGKPRSSDKAPTYEFGFGLSWSTFKFSNLHIQKNNVGPMSPPNGKTIAAPSLGSFSKNLKDYGFPKNVRRIKEFIYPYLSTTTSGKEASGDAHYGQTAKEFLPAGALDGSPQPRSAASGEPGGNRQLYDILYTVTATITNTGSVMDDAVPQLYLSHGGPNEPPKVLRGFDRIERIAPGQSVTFKADLTRRDLSNWDTKKQQWVITDYPKTVYVGSSSRDLPLSARLP

SEQ ID NO: 50 - protein, 861 aa, Aspergillus oryzae
MKLGWIEVAALAAASVVSAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAHHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 51 - protein, 860 aa, Aspergillus niger
MRFTLIEAVALTAVSLASADELAYSPPYYPSPWANGQGDWAQAYQRAVDIVSQMTLDEKVNLTTGTGWELELCVGQTGGVPRLGVPGMCLQDSPLGVRDSDYNSAFPAGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGFGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGYKYYYVSEGPYEKVNQYVNVQRNHSELIRRIGADSTVLLKNDGALPLTGKERLVALIGEDAGSNPYGANGCSDRGCDNGTLAMGWGSGTANFPYLVTPEQAISNEVLKHKNGVFTATDNWAIDQIEALAKTASVSLVFVNADSGEGYINVDGNLGDRRNLTLWRNGDNVIKAAASNCNNTIVVIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASDYLYPSGLQRITKFIYPWLNGTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKLPLRASLPTVH

SEQ ID NO: 52 - protein, 763 aa, Kuraishia capsulata
MKSTIIILSVLAAATAKNISKAEMENLEHWWSYGRSDPVYPSPEISGLGDWQFAYQRAREIVALMTNEEKTNLTFGSSGDTGCSGMISDVPDVDFPGLCLQDAGNGVRGTDMVNAYASGLHVGASWNRQLAYDRAVYMGAEFRHKGVNVLLGPVVGPIGRVATGGRNWEGFTNDPYLAGALVYETTKGIQENVIACTKHFIGNEQETNRNPSGTYNQSVSANIDDKTMHELYLWPFQDSVRAGLGSIMGSYNRVNNSYACKNSKVLNGLLKSELGFQGFVVSDWGGQHTGIASANAGLDMAMPSSTYWEEGLIEAVKNGTVDQSRLDDMATRIIAAWYKYARLDDPGFGMPVSLAEDHELVDARDPAAASTIFQGAVEGHVLVKNENALPLKKPKYISLFGYDGVSTDVNTVGGGFSFFSFDVKAIENKTLISGGGSGTNTPSYVDAPFNAFVAKAREDNTFLSWDFTSAEPVANPASDACIDFINAAASEGYDRPNLADKYSDKLVEAVASQCSNTIVVIHNAGIRLVDNWIEHENVTGVILAHLPGQDTGTSLIEVLYGNQSPSGRLPYTVAKKASDYGGLLWPTEPEGDLDLYFPQSNFTEGVYIDYKYFIQKNITPRYEFGYGLTYTTFDYSELEVDAITNQSYLPPDCTIEEGGAKSLWDIVATVKFTVTNTGDVAAAEVPQLYVGIPNGPPKVLRGFDKKLIHPGQSEEFVFELTRRDLSTWDVVAQNWGLQAGTYQFYVGRSVFDVPLTSALVFTN

SEQ ID NO: 53 - protein, 843 aa, Uromyces fabae
MKTPLGIGSTAAVLYILSNISHVQLATTSPSENQNQSYNPQIEGLTVQPSTVANGLRINSNSLISNFDFEIIQPPPGYEEWTSPVVLPAPVQSGLSPWSESIVRARAFVAQLTIEEKVNLTTGAGTQGRCVGETGTVPRLGFNQPICLQDGPVGIRYTDFNSVFPAAINVAATFDKQLMFKRAQAMAEEFRGKGANVVLAPMTNLMRTPQAGRAWEGYGSDPYLSGVATVQSVLGIQSTRASACVKHYIGNEQEHYRGGSGATASSSNIDDRTLRELYEWPFAEAIHAGVDYIMCSYNRVNQTYACENSKLINGIAKGEHKFQGVMVTDWAAAESGVRTALAGTDMNMPGFMAYGQPSEPNPSTANGSYWGLRMIEAVKNGTVPMERLDDMVTRVISTYYKQGQDKSDYPKLNFMSMGQGTPAEQAVSNHHVNVQKDHYLIIRQIATASTILLKNVNHTLPLKSPDKMRSVVVVGSDAGDNPQGPNSCVDRGCNRGILAIGWGSGTANFAHLTAPATSIQNYLLQSNPTITYRSIFDDYAYDEIAKAASTADVSIVHVSSDSGEGYLTVEGNQGDRSNTSLWNKGDELILKAAEACNNVVVVIHSVGPVDMEAWINHPNVTAVLLAGLPGQEAGSAEVDVLWGSTNPSGRLPYTIAKKPSDYPAELLYESNMTVPQINYSERLNIDYRHFDTYNIEPRFEFGFGLSYTTFAWNSLKFSSSFQLQKTSPVIVPPNLDLYQDVIEFEFQVTNSGPFDGSEVAQLYVDFPNQVNEPPKVLRGFERAYIPSKQSKTIEIKLRVKDLSFWDVITQSWQIPDGKFNFMIGSSSRKIIFTQEISLQHSHM

SEQ ID NO: 54 - protein, 880 aa, Saccharomycopsis fibuligera
MLLILELLVLIIGLGVALPVQTHNLTDNQGFDEESSQWISPHYYPTPQGGRLQGVWQDAYTKAKALVSQMTIVEKVNLTTGTGWQLGPCVGNTGSVPRFGIPNLCLQDGPLGVRLTDFSTGYPSGMATGATFNKDLFLQRGQALGHEFNSKGVHIALGPAVGPLGVKARGGRNFEAFGSDPYLQGIAAAATIKGLQENNVMACVKHFIGNEQDIYRQPSNSKVDPEYDPATKESISANIPDRAMHELYLWPFADSIRAGVGSVMCSYNRVNNTYSCENSYMINHLLKEELGFQGFVVSDWAAQMSGAYSAISGLDMSMPGELLGGWNTGKSYWGQNLTKAVYNETVPIERLDDMATRILAALYATNSFPTKDRLPNFSSFTTKEYGNEFFVDKTSPVVKVNHFVDPSNDFTEDTALKVAEESIVLLKNEKNTLPISPNKVRKLLLSGIAAGPDPKGYECSDQSCVDGALFEGWGSGSVGYPKYQVTPFEEISANARKNKMQFDYIRESFDLTQVSTVASDAHMSIVVVSAVSGEGYLIIDGNRGDKNNVTLWHNSDNLIKAVAENCANTVVVITSTGQVDVESFADHPNVTAIVWAGPLGDRSGTAIANILFGNANPSGHLPFTVAKSNDDYIPIVTYNPPNGEPEDNTLAEHDLLVDYRYFEEKNIEPRYAFGYGLSYNEYKVSNAKVSAAKKVDEELPQPKLYLAEYSYNKTEEINNPEDAFFPSNARRIQEFLYPYLDSNVTLKDGNYEYPDGYSTEQRTTPIQPGGGLGGNDALWEVAYKVEVDVQNLGNSTDKFVPQLYLKHPEDGKFETPVQLRGFEKVELSPGEKKTVEFELLRRDLSVWDTTRQSWIVESGTYEALIGVAVNDIKTSVLFTI

SEQ ID NO: 55 - protein, 876 aa, Saccharomycopsis fibuligera
MLMIVQLLVFALGLAVAVPIQNYTQSPSQRDESSQWVSPHYYPTPQGGRLQDVWQEAYARAKAIVGQMTIVEKVNLTTGTGWQLDPCVGNTGSVPRFGIPNLCLQDGPLGVRFADFVTGYPSGLATGATFNKDLFLQRGQALGHEFNSKGVHIALGPAVGPLGVKARGGRNFEAFGSDPYLQGTAAAATIKGLQENNVMACVKHFIGNEQEKYRQPDDINPATNQTTKEAISANIPDRAMHALYLWPFADSVRAGVGSVMCSYNRVNNTYACENSYMMNHLLKEELGFQGFVVSDWGAQLSGVYSAISGLDMSMPGEVYGGWNTGTSFWGQNLTKAIYNETVPIERLDDMATRILAALYATNSFPTEDHLPNFSSWTTKEYGNKYYADNTTEIVKVNYNVDPSNDFTEDTALKVAEESIVLLKNENNTLPISPEKAKRLLLSGIAAGPDPIGYQCEDQSCTNGALFQGWGSGSVGSPKYQVTPFEEISYLARKNKMQFDYIRESYDLAQVTKVASDAHLSIVVVSAASGEGYITVDGNQGDRKNLTLWNNGDKLIETVAENCANTVVVVTSTGQINFEGFADHPNVTAIVWAGPLGDRSGTAIANILFGKANPSGHLPFTIAKTDDDYIPIETYSPSSGEPEDNHLVENDLLVDYRYFEEKNIEPRYAFGYGLSYNEYEVSNAKVSAAKKVDEELPEPATYLSEFSYQNAKDSKNPSDAFAPADLNRVNEYLYPYLDSNVTLKDGNYEYPDGYSTEQRTTPNQPGGGLGGNDALWEVAYNSTDKFVPQGNSTDKFVPQLYLKHPEDGKFETPIQLRGFEKVELSPGEKKTVDLRLLRRDLSVWDTTRQSWIVESGTYEALIGVAVNDIKTSVLFTI

SEQ ID NO: 56 - protein, 870 aa, Coccidioides immitis
MSPTIWIATLLYWFAFQARKSVAAPPGVGALDDRAELPDGFHSPQYYPAPRGLGAGMEEAYSKAHTVVSKMTLAGKVNLTTGTGFLMALVGQTGSALRFGIPRLCLQDGPLGLRNTDHNTAFPAGISVGATFDKKLMYERGCAMGEEFRGKGANVHLGPSVGPLGRKPRGGRNWEGFGSDPSLQAIAAVETIKGVQSKGVIATIKHLVGNEQEMYRMTNIVQRAYSANIDDRTMHELYLWPFAESVRAGVGAVMMAYNDVNGSASCQNSKLINGILKDELGFQGFVMTDWYAQIGGVSSALAGLDMSMPGDGSVPLSGTSFWASELSRSILNGTVALDRLNDMVTRIVATWFKFGQDKDFPLPNFSSYTQNAKGLLYPGALFSPLGVVNQFVNVQADHHKLARVIARESITLLKNEDNLLPLDPNRAIKYSEQMPGTNPRGINACPDKGCNKGVLTMGWGSGTSNLPYLVTPEDAIRNISKNTEFHITDKFPNNVQPGPDDVAIVFVNADSGENYIIVESNPGDRTVAQMKLWHNGDELIESAAKKFSNVVVVVVHTVGPIIMEKWIDLLRSRVSCLPDFQDKKLEILLLISCSETSVRVAASIYDTESRIGLSDSVSLINQRFGQIQDTFTEGLFIDYRHFQKENITPRYHFGYGLSYTTFNFTEPRLESVTTLSEYPPARKPKAGDRHTPTISHLLQKWPGPKTLTGSGAYLYPYLDNPSAIKPKPGYPYPEAIQPNLNLNPRAGGSEAVTRRYGMLRSRFPLKLLILERNPVRAVAQLYVELPTDDEHPTPKLQLRQFEKTATLEPGQSEVLKMEITRKDVSIWDTMVQDWKVPATGKGIKLWIGASVGDLKAVCETGKGKSCHVLN

SEQ ID NO: 57 - protein, 867 aa, unknown
  Piromyces sp. E2
MKIQNILVALTCGLVSQVFATSWSEADEKAKSFMSDLSESEKIDIVTGYMNMQGTCVGNIKPLDRKNFKGLCLQDGPAGVRFNGGTSTTWQAGINNAATFNKDLLYKIGKDQGAEFYAKGINIALAPSMNILRAPASGRVWENFGEDPYLSGVCGAQITKGYQDSGVIVAAKHYVANDIEHNREASSSNMDDQTLMEIHVEPFYRTIKDGDAGSVMASYNAVNNIYVVQNKKVLTEILKEGIGFQGFVMSDWWAIHDLEGSFNAGMDMNMPGGKAWGPDYVNNSFWGSNISNAIRSGQVSSSRLDDAVRRIIRTLYRFDQMSGYPNVNLKAPSMHADTNRQAAIESSVLLKNADDILPLTKKYRKIAIIGKDADKAQSCTDTACSGGNIIQGWGSGTTDFTGISDPITAIKNRASKEGISIVSSISDSANEGANVAKDADVAVVFVRATSGEEYIVVDNNKGDRNNLDLWHGGNDLVKSVAAVNKNTVVVIHAPATVNLPFLNNVKAIIHAGMPGAESGNAIASILFGDSNPSGHLPFTWAAREDYCCDVSYPAELPHGGNSKTAYDYKEGLFVGYRWFDKKNKTPIFPFGHGLSYTTFDYSNLSVSLKKSGTQVTGLEATVTVANTGSYEGATVPMLFLGFPAVSELGDYPVRNLKAFEKVNLKAGEKKTVTLTVDQHGLSYYNTSKKSFVVPTGGEFTVYVGKSAGDLPLKKAIKNTQGTNESSSSVGDENNNNPNNNADCSVNGYKCCSNSNAEVVYTDGDGNWGVENGQWCIIKEQQQQQTCFSIKLGYPCCKGNEVAYTDNDGQWGFENGQWCGIATATSGAGGCPYTSKNGYPVCQTTTKVEYVDSDKWGVENGNWCIMCN

SEQ ID NO: 58 - protein, 825 aa, Hansenula anomala
MLLPLYGLASFLVLSQAALVNTSAPQASNDDPFNHSPSFYPTPQGGRINDGKWQAAFYRARELVDQMSIAEKVNLTTGVGSASGPCSGNTGSVPRLNISSICVQDGPLSVRAADLTDVFPCGMAASSSFNKQLIYDRAVAIGSEFKGKGADAILGPVYGPMGVKAAGGRGWEGHGPDPYLEGVIAYLQTIGIQSQGVVSTAKHLIGNEQEHFRFAKKDKHAGKIDPGMFNTSSSLSSEIDDRAMHEIYLWPFAEAVRGGVSSIMCSYNKLNGSHACQNSYLLNYLLKEELGFQGFVMTDWGALYSGIDAANAGLDMDMPCEAQYFGGNLTTAVLNGTLPQDRLDDMATRILSALIYSGVHNPDGPNYNAQTFLTEGHEYFKQQEGDIVVLNKHVDVRSDINRAVALRSAVEGVVLLKNEHETLPLGREKVKRISILGQAAGDDSKGTSCSLRGCGSGAIGTGYGSGAGTFSYFVTPADGIGARAQQEKISYEFIGDSWNQAAAMDSALYADAAIEVANSVAGEEIGDVDGNYGDLNNLTLWHNAVPLIKNISSINNNTIVIVTSGQQIDLEPFIDNENVTAVIYSSYLGQDFGTVLAKVLFGDENPSGKLPFTIAKDVNDYIPVIEKVDVPDPVDKFTESIYVDYRYFDKYNKPVRYEFGYGLSYSNFSLSDIEIQTLQPFSENAEPAANYSETYQYKQSNMDPSEYTVPEGFKELANYTYPYIHDASSIKANSSYDYPEGYSTEQLDGPKSLAAGGLGGNHTCGMLVTLSLLKSQIKVLMLVGLHLNCMLDIQIMMNSQHLQCNYVDLKRCFWIKIILKLFLLN